(19) United States
(12) Patent Application Publication
(10) Pub. No.: US 2008/0312192 A1
(43) Pub. Date: Dec. 18, 2008

(54) Diaryl Urea Derivatives in the Treatment of Protein Kinase Dependent Diseases

(76) Inventors: Guido Bold, Gipf-Oberfrick (CH); Giorgio Caravatti, Bottmingen (CH); Andreas Floersheimer, Dornach (CH); Vito Guagnano, Basel (CH); Patricia Imbach, Kaiseraugst (CH); Keiichi Masuya, Ibaraki Pref. (JP); Johannes Roesel, Riehen (CH); Andrea Vaupel, Riehen (CH); Carlos Garcia-Echeverria, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/580,259

(22) PCT Filed: Nov. 26, 2004
(86) PCT No.: PCT/EP2004/013459

§ 371 Date: Aug. 25, 2008

(30) Foreign Application Priority Data
Nov. 28, 2003 (GB) 0327734.0
Aug. 10, 2004 (GB) 0417805.9

(51) Int. Cl. A61K 31/5377; A61K 31/496; A61K 31/513; A61K 31/655; A61K 31/506

(52) U.S. Cl. 514/150; 514/210.2; 514/252.14; 514/269; 514/235.8; 514/272

(57) ABSTRACT

The invention relates to the use of diaryl urea derivatives for the manufacture of pharmaceutical compositions for the treatment of RET dependent disorders, especially RET dependent tumour diseases. The invention further relates to novel N-[4-(pyrimidin-4-yloxy)-phenyl]-N′-phenyl-urea derivatives and their use in the treatment of the animal or human body, especially in the treatment of a protein kinase dependent disease, to pharmaceutical compositions comprising such novel N-[4-(pyrimidin-4-yloxy)-phenyl]-N′-phenyl-urea derivatives and to the use of such novel N-[4-(pyrimidin-4-yloxy)-phenyl]-N′-phenyl-urea derivatives for the preparation of pharmaceutical compositions for use in the treatment of protein kinase dependent diseases, especially of proliferative diseases, such as tumour diseases.

### SUMMARY OF THE INVENTION

The invention relates to the use of diaryl urea derivatives for the manufacture of pharmaceutical compositions for the treatment of RET dependent disorders, especially RET dependent tumour diseases. The invention further relates to novel N-[4-(pyrimidin-4-yloxy)-phenyl]-N′-phenyl-urea derivatives and their use in the treatment of the animal or human body, especially in the treatment of a protein kinase dependent disease, to pharmaceutical compositions comprising such novel N-[4-(pyrimidin-4-yloxy)-phenyl]-N′-phenyl-urea derivatives and to the use of such novel N-[4-(pyrimidin-4-yloxy)-phenyl]-N′-phenyl-urea derivatives for the preparation of pharmaceutical compositions for use in the treatment of protein kinase dependent diseases, especially of proliferative diseases, such as tumour diseases.

### BACKGROUND OF THE INVENTION

Protein kinases (PKs) are enzymes which catalyze the phosphorylation of specific serine, threonine or tyrosine residues in cellular proteins. These post-translational modifications of substrate proteins act as molecular switch regulating cell proliferation, activation and/or differentiation. Aberrant or excessive wild-type or mutated PK activity has been observed in many disease states including benign and malignant proliferative disorders. In many cases, it has been possible to treat diseases, such as proliferative disorders, by making use of PK inhibitors.

In view of the large number of protein kinases and the multitude of proliferative and other PK-related diseases, there is an ever-existing need to provide compounds that are useful as PK inhibitors and thus in the treatment of these PK related diseases.

### GENERAL DESCRIPTION OF THE INVENTION

The rearranged during transfection (RET) proto-oncogene was identified as the susceptibility gene for multiple endocrine neoplasia type 2 (MEN 2), an inherited cancer syndrome characterized by medullary thyroid carcinoma (MTC) (reviewed in Eng, J. Clin. Oncol., 17, 380-93, 1999; Takahashi, Cytokine and Growth Factor Revs., 12, 361-73, 2001). The subtype RET/MEN2A is characterized by mutations in the extra-cellular domain (e.g. C634R) which lead to constitutive dimerization and activation of the kinase. The less prevalent subtype RET/MEN2B is characterized by a mutation in the activation loop (M918T) which leads to constitutive activation and altered substrate specificity. RET/MEN2B remains responsive to its ligands, and therefore, temporal and spatial expression of the neurotropic factors of GDNF family may further influence the clinical phenotypes of MEN 2B patients (reviewed in Jhiang, Oncogene, 19, 5590-7, 2000).

Papillary thyroid carcinoma (PTC) is the most common type (85%) of the thyroid malignancy (Lorentz, World Journal of Surgery, 18, 547-50, 1994). The tumour is associated with somatic mutations of RET proto-oncogene, which is activated by gene rearrangements (Pacini, J. Endocrin. Invest., 23, 328-38, 2000; Tallini and Asa, Adv. Anat. Pathol., 8, 345-54, 2001). The rearranged proto-oncogene, PTC oncogene (RET/PTC) is the product of the fusion of the tyrosine-kinase domain of the proto-RET to other genes. The three most common variants are RET/PTC1, RET/PTC2 and RET/PTC3 (Pacini, J. Endocrin. Invest., 23, 328-38, 2000; Tallini and Asa, Adv. Anat. Pathol., 8, 345-54, 2001). In RET/PTC1 RET/PTC2 and RET/PTC3 the tyrosine-kinase domain fuses with the genes H4, R1α and ELE1, respectively (Tallini and Asa, Adv. Anat. Pathol., 8, 345-54, 2001).

The various mutated forms of the RET receptor tyrosine kinase are therefore attractive targets for the development of drugs targeting cancer; specially thyroid cancer.

RET and the various mutated forms thereof have also been found to be expressed at the protein and/or mRNA level in many different tumour cell lines and tissues. Inhibitors of wild-type and mutated RET, are therefore also especially appropriate in the treatment of other RET dependent cancers such as RET dependent cancers of the colon, lung, breast and pancreas as well as other RET dependent solid tumours and leukemias.

It was now found that the compounds of formula I are inhibitors of wild-type and/or mutated RET. These compounds are therefore useful in the treatment of RET dependent diseases, especially RET dependent proliferative diseases, in particular RET dependent tumour diseases, such as RET dependent cancers of the colon, lung, breast and pancreas as well as other RET dependent solid tumours and leukemias and especially RET dependent thyroid cancer.

### DETAILED DESCRIPTION OF THE INVENTION

The invention relates to the use of diaryl urea derivatives that are compounds of formula I

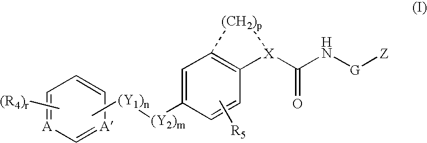

wherein G is either not present, lower alkylene or C₃-C₅cycloalkylene and Z is a radical of the formula Ia

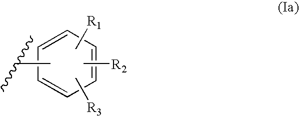

or G is not present and Z is a radical of the formula Ib

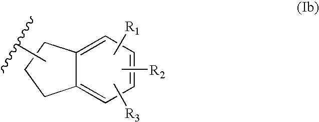

A is CH, N or N→O and A′ is N or N→O, with the proviso that not more than one of A and A′

### can be N→O;

n is 1 or 2;
m is 0, 1 or 2;
p is 0, 2 or 3;
r is 0 to 5;
X is NR if p is 0, wherein R is hydrogen or an organic moiety, or if p is 2 or 3, X is nitrogen which together with (CH₂)ₚ and the bonds represented in dotted (interrupted) lines (including the atoms to which they are bound) forms a ring,
or
X is CHK wherein K is lower alkyl or hydrogen and p is zero,
with the proviso that the bonds represented in dotted lines, if p is zero, are absent;

### Y₁ is O, S or CH₂;

### Y₂ is O, S or NH;

with the proviso that (Y₁)ₙ—(Y₂)ₘ does not include O—O, S—S, NH—O, NH—S or S-0 groups;
each of R₁, R₂, R₃ and R₅, independently of the others, is hydrogen or an inorganic or organic moiety or any two of them together form a lower alkylene-dioxy bridge bound via the oxygen atoms, and the remaining one of these moieties is hydrogen or an inorganic or organic moiety;
and R₄; (if present, that is, if r is not zero) is an inorganic or organic moiety;
or a tautomer thereof;
or a pharmaceutically acceptable salt thereof;
for the manufacture of pharmaceutical compositions for use in the treatment of RET dependent diseases.

The present invention further relates to novel N-[4-(pyrimidin-4-yloxy)-phenyl]-N′-phenyl-urea derivatives of formula I as disclosed in the Examples hereinbelow (Examples 1-70) which are hereinafter called ‘NOVEL COMPOUNDS OF THE INVENTION’). The NOVEL COMPOUNDS OF THE INVENTION especially show inhibition of one or more of the following protein tyrosine kinases: c-Abl, Bcr-Abl, the receptor tyrosine kinases Flt-3, RET, vascular endothelial growth factor receptor (VEGF-R) and Tek (Tie2), especially Flt-3, as well as combinations of two or more of these; the NOVEL COMPOUNDS OF THE INVENTION are further also appropriate for the inhibition of the non-receptor tyrosine kinase Raf, and/or for the inhibition of mutants of these enzymes, especially of Bcr-Abl, for example the Glu255->Lysine mutant. In view of these activities, the NOVEL COMPOUNDS OF THE INVENTION can be used for the treatment of diseases related to especially aberrant or excessive activity of such types of kinases, especially those mentioned.

The general terms used hereinbefore and hereinafter preferably have, within this disclosure, the following meanings, unless otherwise indicated:

Where “the use of diaryl urea derivatives for the manufacture of pharmaceutical compositions for use in the treatment of RET dependent diseases” is mentioned, this is meant to include also the use of such diaryl urea derivatives in the treatment of RET dependent diseases, methods of use of such diaryl urea derivatives in the treatment of RET dependent diseases and pharmaceutical compositions comprising such diaryl urea derivatives for the treatment of RET dependent diseases. It is further also meant to include the diaryl urea derivatives for use in the treatment of RET dependent diseases.

The prefix “lower” denotes a radical having 1 up to and including a maximum of 7, especially 1 up to and including a maximum of 4 carbon atoms, the radicals in question being either linear or branched with single or multiple branching. Lower alkyl, for example, is methyl, ethyl, n-propyl, sec-propyl, n-butyl, isobutyl, sec-butyl, tert-butyl, n-pentyl, n-hexyl or n-heptyl.

Where the plural form is used for compounds, salts, pharmaceutical compositions, diseases and the like, this is intended to mean also a single compound, salt, or the like.

Halo(geno) is preferably Iodo, bromo, chloro or fluoro, especially fluoro, chloro or bromo.

In view of the close relationship between the diaryl urea derivatives in free form and in the form of their salts, including those salts that can be used as intermediates, for example in the purification or identification of the compounds of formula I, tautomers or tautomeric mixtures and their salts, any reference hereinbefore and hereinafter to these compounds, especially to the NOVEL COMPOUNDS OF THE INVENTION, is to be understood as referring also to the corresponding tautomers of these compounds, tautomeric mixtures of these compounds, N-oxides of these compounds, or salts of any of these, as appropriate and expedient and if not mentioned otherwise. Tautomers can, e.g., be present in cases where amino or hydroxy, each with a least one bound hydrogen, are bound to carbon atoms that are bound to adjacent atoms by double bonds (e.g. keto-enol or imine-enamine tautoemerism). Preferred tautomers are the pyridin-on-yl or pyrimidin-on-yl forms of compounds wherein R₄ is hydroxy and the other moieties are defined as for compounds of the formula I.

Where “a compound . . . , a tautomer thereof; or a salt thereof” or the like is mentioned, this means “a compound . . . a tautomer thereof, or a salt compound or the tautomer”.

Asymmetric carbon atoms of a compound of formula I that are optionally present may exist in the (R), (S) or (R,S) configuration, preferably in the (R) or (S) configuration. Substituents at a double bond or a ring may be present in cis-(=Z-) or trans (=E-) form. The compounds may thus be present as mixtures of isomers or preferably as pure isomers.

Salts are preferably the pharmaceutically acceptable salts of the diaryl urea derivatives of the present invention, especially of the NOVEL COMPOUNDS OF THE INVENTION.

Salt-forming groups are groups or radicals having basic or acidic properties. Compounds having at least one basic group or at least one basic radical, for example amino, a secondary amino group not forming a peptide bond or a pyridyl radical, may form acid addition salts, for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acids hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethanesulfonic acid, or aromatic sulfonic acids, for example benzene-, p-toluene- or naphthalene-2-sulfonic acid. When several basic groups are present mono- or poly-acid addition salts may be formed.

Compounds having acidic groups, a carboxy group or a phenolic hydroxy group, may form metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri-(2-hydroxyethyl)-amine, or heterocyclic bases, for example N-ethyl-piperidine or N,N′-dimethylpiperazine. Mixtures of salts are possible.

Compounds having both acidic and basic groups can form internal salts.

For the purposes of isolation or purification, as well as in the case of compounds that are used further as intermediates, it is also possible to use pharmaceutically unacceptable salts, e.g. the picrates. Only pharmaceutically acceptable, non-toxic salts may be used for therapeutic purposes, however, and those salts are therefore preferred.

An organic moiety R is preferably unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl or unsubstituted or substituted cycloalkenyl; preferred is unsubstituted alkyl.

“Substituted”, wherever used for a moiety, means that one or more hydrogen atoms in the respective moiety, especially up to 5, more especially up to three, of the hydrogen atoms are replaced independently of each other by the corresponding number of substituents which preferably are independently selected from the group consisting of lower alkyl, for example methyl, ethyl or propyl, halo-lower alkyl, for example trifluoromethyl, C₅-C₁₆-aryl, especially phenyl or naphthyl (where C₆-C₁₆-aryl, especially phenyl or napthyl, is unsubstituted or substituted by one or more, especially up to three moieties selected from halogen, carboxy, lower alkoxycarbonyl, hydroxy, lower alkoxy, phenyl-lower alkoxy, lower alkanoyloxy, lower alkanoyl, amino, N-lower alkylamino, N,N-di-lower alkylamino, N-phenyl-lower alkylamino, N,N-bis(phenyl-lower alkyl)-amino, lower alkanoylamino, halo, halo-lower alkyl, e.g. trifluoromethyl, sulfo, sulfamoyl, carbamoyl, N-lower alkyl-carbamoyl, N-(hydroxy-lower alkyl)-carbamoyl, such as N-(2-hydroxyethyl)-carbamoyl, cyano, cyano-lower alkyl and nitro), C₃-C₁₀-cycloalkyl, especially cyclopropyl or cyclohexyl, hydroxy-C₃-C₁₀-cycloalkyl, such as hydroxycyclohexyl, heterocyclyl with 5 or 6 ring atoms and 1 to 3 ring heteroatoms selected from O, N and S, especially piperidinyl, especially piperidin-1-yl, piperazinyl, especially piperazin-1-yl, morpholinyl, especially morpholin-1-yl, hydroxy, lower alkoxy, for example methoxy, halo-lower alkoxy, especially 2,2,2-trifluoroethoxy, phenyl-lower alkoxy, amino-lower alkoxy, such as 2-eminoethoxy; lower alkanoyloxy, hydroxy-lower alkyl, such as hydroxymethyl or 2-hydroxyethyl, amino, N-lower alkylamino, N,N-di-lower alkylamino, N-phenyl-lower alkylamino, N,N-bis(phenyl-lower alkyl)-amino, lower alkanoylamino, especially acetylamino, benzoylamino, carbamoyl-lower alkoxy, N-lower alkylcarbamoyl-lower alkoxy or N,N-di-lower alkylcarbamoyl-lower alkoxy, amidino, N-hydroxy-am-idino, guanidino, amino-lower alkyl, such as aminomethyl or 2-aminoethyl, amidino-lower alkyl, such as 2-aminoethyl, N-hydroxyamidino-lower alkyl, such as N-hydroxy-amidino-methyl or -2-ethyl, halogen, for example fluoro, chloro, bromo or iodo, carboxy, lower alkoxycarbonyl, phenyl-, naphthyl- or fluorenyl-lower alkoxycarbonyl, such as benzyloxycarbonyl, lower alkanoyl, sulfo, lower alkanesulfonyl, for example methanesulfonyl (CH₃—S(O)₂—), phosphono (—P(═O)(OH)₂), hydroxy-lower alkoxy phosphoryl or di-lower alkoxyphosphoryl, carbamoyl, mono- or di-lower alkylcarbamoyl, mono- or di-(hydroxy-lower alkyl)-carbamoyl, sulfamoyl, mono- or di-lower alkylaminosulfonyl, nitro, cyano-lower alkyl, such as cyanomethyl, and cyano. It goes without saying that substitutents are only at positions where they are chemically possible, the person skilled in the art being able to decide (either experimentally or theoretically) without inappropriate effort which substitutions are possible and which are not. For example, amino or hydroxy groups with free hydrogen may be unstable if bound to carbon atoms with unsaturated (e.g. olefinic) bonds.

Alkyl preferably has up to 20, more preferably up to 12 carbon atoms and is linear or branched one or more times; preferred is lower alkyl, especially C₁-C₄-alkyl, in particular methyl, ethyl or n-propyl. Alkyl is unsubstituted or substituted, preferably by one or more substituents independently selected from those mentioned above under “Substituted”. Unsubstituted alkyl, preferably lower alkyl, is especially preferred as an organic moiety R.

Among the moieties corresponding to substituted alkyl, hydroxy-lower alkyl, especially 2-hydroxyethyl, and/or halo-lower alkyl, especially trifluoromethyl or 2,2,2-trifluoroethyl, are especially preferred.

Alkenyl is preferably a moiety with one or more double bonds and preferably has 2 to 20, more preferably up to 12, carbon atoms; it is linear or branched one or more times (as far as possible in view of the number of carbon atoms). Preferred is C₂-C₇-alkenyl, especially C₃-C₄-alkenyl, such as allyl or crotyl. Alkenyl can be unsubstituted or substituted, especially by one or more, more especially up to three, of the substituents mentioned above under “substituted”. Substituents such as amino or hydroxy (with free dissociable hydrogen) preferably are not bound to carbon atoms that participate at a double bond, and also other substituents that are not sufficiently stable are preferably excluded. Unsubstituted alkenyl, in particular C₂-C₇-alkenyl, is preferred.

Alkynyl is preferably a moiety with one or more triple bonds and preferably has 2 to 20, more preferably up to 12, carbon atoms; it is linear of branched one or more times (as far as possible in view of the number of carbon atoms). Preferred is C₂-C₇-alkynyl, especially C₃-C₄-alkynyl, such as ethinyl or propin-2-yl. Alkynyl can be unsubstituted or substituted, especially by one or more, more especially up to three, of the substituents mentioned above under “substituted”. Substituents such as amino or hydroxy (with free dissociable hydrogen) preferably are not bound to carbon atoms that participate at a triple bond, and also other substituents that are not sufficiently stable are preferably excluded. Unsubstituted alkynyl, in particular C₂-C₇-alkynyl, is preferred.

Aryl preferably has a ring system of not more than 16 carbon atoms, is preferably mono-, bi- or tric-cyclic, and is unsubstituted or substituted preferably as defined above under “Substituted”. Preferably, aryl is selected from phenyl, naphthyl, indenyl, azulenyl and anthryl, and is preferably in each case unsubstituted or lower alkyl, especially methyl, ethyl or n-propyl, halo (especially fluoro, chloro, bromo or iodo), halo-lower alkyl (especially trifluoromethyl), hydroxy, lower alkoxy (especially methoxy), halo-lower alkoxy (especially 2,2,2-trifluoroethoxy), amino-lower alkoxy (especially 2-amino-ethoxy), lower alkyl (especially methyl or ethyl) carbamoyl, N-(hydroxy-lower alkyl)-carbamoyl (especially N-(2-hydroxyethyl)-carbamoyl) and/or sulfamoyl-substituted aryl, especially a corresponding substituted or unsubstituted phenyl.

Heterocyclyl is preferably a heterocyclic radical that is unsaturated, saturated or partially saturated in the bonding ring and is preferably a monocyclic or in a broader aspect of the invention bicyclic or tricyclic ring; has 3 to 24, more preferably 4 to 16 ring atoms; wherein at least in the ring bonding to the radical of the molecule of formula I one or more, preferably one to four, especially one or two carbon ring atoms are replaced by a heteroatom selected from the group consisting of nitrogen, oxygen and sulfur, the bonding ring preferably having 4 to 12, especially 5 to 7 ring atoms; heteroaryl being unsubstituted or substituted by one or more, especially 1 to 3, substitutents independently selected from the group consisting of the substituents defined above under “substituted”; especially being a heteroaryl radical selected from the group consisting of oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl, especially piperidin-1-yl, piperazinyl, especially piperazin-1-yl, pyridazinyl, morpholinyl, especially morpholino, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being-unsubstituted or substituted by one to two radicals selected from the group consisting of lower alkyl, especially methyl or tert-butyl, lower alkoxy, especially methoxy, and halo, especially bromo or chloro. Unsubstituted heterocyclyl, especially piperidyl, piperazinyl, thiomorpholino or morpholino, is preferred.

Cycloalkyl is preferably C₃-C₁₀-cycloalkyl, especially cyclopropyl, dimethylcyclopropyl, cyclobutyl, cyclopentyl, cyclohexyl or cycloheptyl, cycloalkyl being unsubstituted or substituted by one or more, especially 1 to 3, substitutents independently selected from the group consisting of the substituents defined above under “Substituted”.

Cycloalkenyl is preferably C₅-C₁₀-cycloalkenyl, especially cyclopentenyl, cyclohexenyl or cycloheptenyl, cycloalkenyl being unsubstituted or substituted by one or more, especially 1 to 3, substitutents independently selected from the group consisting of the substituents defined above under “Substituted”.

An inorganic moiety is preferably halogen, hydroxy, amino, or nitro.

The bonds represented by dotted (interrupted) lines and binding (CH₂)ₚ, are present if p is 2 or 3, or absent if p is zero.

An organic moiety is preferably unsubstituted or substituted alkyl, unsubstituted or substituted alkenyl, unsubstituted or substituted alkynyl, unsubstituted or substituted unsubstituted or substituted aryl, unsubstituted or substituted heterocyclyl, unsubstituted or substituted cycloalkyl or unsubstituted or substituted cycloalkenyl, unsubstituted or substituted alkoxy, unsubstituted or substituted alkenyloxy, unsubstituted or substituted alkynyloxy, unsubstituted or substituted aryloxy, unsubstituted or substituted heterocyclyloxy, unsubstituted or substituted cycloalkoxy or unsubstituted or substituted cycloalkenyloxy, or unsubstituted or substituted alkylamino, unsubstituted or substituted alkenylamino, unsubstituted or substituted alkynylamino, unsubstituted or substituted arylamino, unsubstituted or substituted heterocyclylamino, unsubstituted or substituted cycloalkylamino or unsubstituted or substituted cycloalkenylamino.

An organic moiety is preferably alkyl, especially lower alkyl, such as methyl, ethyl or propyl, halo-lower alkyl, such as trifluoromethyl, lower alkoxy, such as methoxy, halo-lower alkoxy, such as 2,2,2-trifluoroethoxy, halo, such as chloro or bromo, phenyl, phenylamino, hydroxyphenyl-amino, such as 4-hydroxyphenylamino, amino-lower alkyl-oxyphenylamino, such as [4-(2-aminoethyl)oxy]-phenyl-amino, carbamoylphenyl-amino, such as 4-sulfamoyl-phenyl-amino, [N-hydroxy-lower alkyl)-carbamoyl]-phenyl-amino, such as {N-[4-(2-hydroxyethyl)-carbamoyl]-phenyl}-amino, 5- or 6-membered saturated heterocyclyl with 1 or 2 heteroatoms selected from the group consisting of N, O and S, especially piperidyl, such as piperidin-1-yl, piperazinyl, such as piperazin-1-yl, morpholinyl, such as morpholino, or further thiomorpholinyl, such as thiomorpholino.

A basic organic moiety is a moiety selected from the definition of an organic moiety as given herein and having basic (alkaline) properties. Preferably a basic organic moiety is piperidyl, especially piperidin-1-yl, piperidyl-lower-alkyl, especially piperidin-1-ylmethyl, lower alkyl-piperazinyl, especially 4-methyl-piperazin-1-yl or 4-ethyl-piperazin-1-yl, or lower alkyl-piperazinyl-lower alkyl, especially 4-methyl-piperazin-1-ylmethyl or 4-ethyl-piperazin-1-ylmethyl.

If any two of R₁, R₂ and R₃ together form a lower alkylene-dioxy bridge bound via the oxygen atoms said bridge is preferably methylendioxy (O—CH₂O) or ethylendioxy (O—CH₂—CH₂—O) bound via the oxygen atoms to vicinal carbon atoms, and the remaining one of these moieties is hydrogen or an inorganic or organic moiety as described above.

The term “treatment of tyrosine protein kinase dependent diseases” refers to the prophylactic or preferably therapeutic (including palliative and/or curing) treatment of said diseases, especially of the diseases mentioned herein.

The compounds of formula I have valuable pharmacological properties and are useful in the treatment of RET dependent diseases, especially RET dependent proliferative diseases, in particular RET dependent tumour diseases, such as RET dependent cancers of the colon, lung, breast and pancreas as well as other RET dependent solid tumours and leukemias and especially RET dependent thyroid cancer.

RET kinase inhibition is determined as follows:

Cloning and expression: The baculovirus donor vector pFB-GSTX3 is used to generate a recombinant baculovirus that expresses the amino acid region 658-1072 (Swiss prot No. Q9BTB0) of the cytoplasmic kinase domain of human RET-Men2A which corresponds to the wild-type kinase domain of RET (wtRET) and RET-Men2B, which differs from the wtRET by the activating mutation in the activation loop M918T. The coding sequence for the cytoplasmic domain of wtRET is amplified by PCR from a cDNA library using specific primers. RET-Men2B is generated through site-directed mutagenesis resulting in the M918T mutation. The amplified DNA fragments and the pFB-GSTX3 vector are made compatible for ligation by digestion with SalI and KpnI. Ligation of these DNA fragments results in the baculovirus donor plasmids pFB-GX3-RET-Men2A and pFB-GX3-RET-Men2B, respectively.

Production of virus: The baculovirus donor plasmids containing the kinase domains are transfected into the DH10Bac cell line (GIBCO) and the transfected cells are plated on selective agar plates. Colonies without insertion of the fusion sequence into the viral genome (carried by the bacteria) are blue. Single, white colonies are picked and viral DNA (bacmid) is isolated from the bacteria by standard plasmid purification procedures. Sf9 cells or Sf21 cells (American Type Culture Collection) are then transfected in 25 cm² flasks with the viral DNA using Cellfectin reagent.

Protein expression in Sf9 cells: Virus-containing media is collected from the transfected cell culture and used for infection to increase its titer. Virus-containing media obtained after two rounds of infection is used for large-scale protein expression. For large-scale protein expression 100 cm² round tissue culture plates are seeded with 5×10⁷ cells/plate and infected with 1 mL of virus-containing media (approximately 5 MOIs). After 3 days, the cells are scraped off the plate and centrifuged at 500 rpm for 5 minutes. Cell pellets from 10-20, 100 cm² plates are re-suspended in 50 mL of ice-cold lysis buffer (25 mM Tris-HCl, pH 7.5, 2 mM EDTA, 1% NP-40, 1 mM DTT, 1 mM PMSF). The cells are stirred on ice for 15 minutes and then centrifuged at 5,000 rpms for 20 minutes.

Purification of GST-tagged proteins: The centrifuged cell lysate is loaded onto a 2 mL glutathione-sepharose column (Pharmacia) and washed 3× with 10 mL of 25 mM Tris-HCl, pH 7.5, 2 mM EDTA, 1 mM DTT, 200 mM NaCl. The GST-tagged proteins are then eluted by 10 applications (1 mL each) of 25 mM Tris-HCl, pH 7.5, 10 mM reduced-glutathione, 100 mM NaCl, 1 mM DTT, 10% glycerol and stored at −70° C.

Measure of enzyme activity: Tyrosine protein kinase assays with either purified GST-wtRET or GST-RET-Men2B protein are carried out in a final volume of 30 μL containing 15 ng of either GST-RET or GST-RET-Men2B protein, 20 mM Tris-HCl, pH 7.5, 1 mM MnCl₂, 10 mM MgCl₂, 1 mM DTT, 3 μg/mL poly(Glu, Tyr) 4:1, 1% DMSO, 2.0 μM ATP (γ-[³³P]-ATP 0.1 μCi). The activity is assayed in the presence or absence of inhibitors, by measuring the incorporation of ³³P from [γ³³P] ATP into poly(Glu, Tyr) 4:1. The assay is carried out in 96-well plates at ambient temperature for 15 minutes under conditions described above and terminated by the addition of 20 μL of 125 mM EDTA. Subsequently, 40 μL of the reaction mixture are transferred onto Immobilon-PVDF membrane (Millipore) previously soaked for 5 minutes with methanol, rinsed with water, then soaked for 5 minutes with 0.5% H₃PO₄ and mounted on vacuum manifold with disconnected vacuum source. After spotting all samples, vacuum is connected and each well rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μl/well of Microscint™ (Packard). IC₅₀ values are calculated by linear regression analysis of the percentage inhibition of each compound in duplicate, at 4 concentrations (usually 0.01, 0.1, 1 and 10 μM). One unit of protein kinase activity is defined as 1 nmole of ³³P transferred from [γ³³P] ATP to the substrate protein/minute/mg of protein at 37° C. The compounds of formula I here show IC₅₀ values in the range between 0.005 and 5 μM, especially between 0.01 and 1 μM.

Where subsequently the term “USE” is mentioned in connection with the NOVEL COMPOUNDS OF THE INVENTION, this includes any one or more of the following embodiments of the invention, respectively: the use in the treatment of (especially tyrosine) protein kinase dependent diseases, the use for the preparation of pharmaceutical compositions for use in the treatment of said diseases, methods of use of the NOVEL COMPOUNDS OF THE INVENTION in the treatment of said diseases, pharmaceutical compositions comprising NOVEL COMPOUNDS OF THE INVENTION for use in the treatment of said diseases, and NOVEL COMPOUNDS OF THE INVENTION for use in the treatment of said diseases, as appropriate and expedient, if not stated otherwise. In particular, diseases to be treated and are thus preferred for USE of a NOVEL COMPOUND OF THE INVENTION are selected from (especially tyrosine) protein kinase dependent (“dependent” meaning also “supported”, not only “solely dependent”) diseases mentioned below, especially corresponding proliferative diseases, more especially diseases that depend on c-Abl, Bcr-Abl, Flt-3, RET, VEGF-R and/or Tek, especially Flt-3, activity, especially the diseases mentioned below under these specific protein tyrosine kinases. Other kinases that can be inhibited by the NOVEL COMPOUNDS OF THE INVENTION include platelet-derived growth factor receptor (PDGF-R), fibroblast growth factor receptor (FGF-R), insulin-like growth factor I receptor (IGF-IR), Eph receptors such as especially EphB4 receptor, c-Kit, Met, c-Src, Raf and ras.

The NOVEL COMPOUNDS OF THE INVENTION have valuable pharmacological properties and are useful in the treatment of protein kinase dependent diseases, especially of protein tyrosine kinase dependent diseases, for example as drugs to treat proliferative diseases.

The efficacy of the NOVEL COMPOUNDS OF THE INVENTION as inhibitors of c-Abl protein tyrosine kinase activity can be demonstrated as follows:

An in vitro enzyme assay is performed in 96-well plates as a filter binding assay as described by Geissler et al., in Cancer Res. 1992; 52:4492-4498, with the following modifications. The His-tagged kinase domain of c-Abl is cloned and expressed in the baculovirus/Sf9 system as described by Bhat et al. in J. Biol. Chem. 1997; 272:16170-16175. A protein of 37 kD (c-Abl kinase) is purified by a two-step procedure over a Cobalt metal chelate column followed by an anion exchange column with a yield of 1-2 mg/L of Sf9 cells (Bhat et al., reference cited). The purity of the c-Abl kinase is >90% as judged by SDS-PAGE after Coomassie blue staining. The assay contains (total volume of 30 μL): c-Abl kinase (50 ng), 20 mM Tris.HCl, pH 7.5, 10 mM MgCl₂, 10 mM Na₃VO₄, 1 mM DTT and 0.06 μCi/assay [γ³³P]-ATP (5 μM ATP) using 30 μg/mL poly-Ala,Glu,Lys,Tyr-6:2:5:1 (Poly-AEKY, Sigma P1152) in the presence of 1% DMSO. Reactions are terminated by adding 10 μL of 250 mM EDTA and 30 μL of the reaction mixture is transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μL 0.5% H₃PO₄. Membranes are removed and washed on a shaker with 0.5% H₃PO₄ (4 times) and once with ethanol. Membranes are counted after drying at ambient temperature, mounting in Packard TopCount 96-well frame, and addition of 10 μL/well of Microscint™ (Packard). Using this test system, the NOVEL COMPOUNDS OF THE INVENTION show IC₅₀ values of inhibition in the range of 0.001 to 100 μM, usually between 0.05 and 5 μM.

The inhibition of VEGF-induced receptor autophosphorylation can be confirmed with a further in vitro experiments in cells such as transfected CHO cells, which permanently express human VEGF-R₂ receptor (KDR), are seeded in complete culture medium (with 10% fetal calf serum=FCS) in 6-well cell-culture plates and incubated at 37° C. under 5% CO₂-until they show about 80% confluency. The compounds to be tested are then diluted in culture medium (without FCS, with 0.1% bovine serum albumin) and added to the cells. (Controls comprise medium without test compounds). After two hours of incubation at 37° C., recombinant VEGF is added; the final VEGF concentration is 20 ng/ml. After a further five minutes incubation at 37° C., the cells are washed twice with ice-cold PBS (phosphate-buffered saline) and immediately lysed in 100 μl lysis buffer per well. The lysates are then centrifuged to remove the cell nuclei, and the protein concentrations of the supernatants are determined using a commercial protein assay (BIORAD). The lysates can then either be immediately used or, if necessary, stored at −20° C.

A sandwich ELISA is carried out to measure the VEGF-R₂ phosphorylation: a monoclonal antibody to VEGF-R₂ (for example Mab 1495.12.14; prepared by H. Towbin, Novartis or comparable monoclonal antibody) is immobilized on black ELISA plates (OptiPlate™ HTRF-96 from Packard). The plates are then washed and the remaining free protein-binding sites are saturated with 3% TopBlock® (Juro, Cat. # TB232010) in phosphate buffered saline with Tween 20® (polyoxyethylen(20)sorbitane monolaurate, ICI/Uniquema) (PBST). The cell lysates (20 μg protein per well) are then incubated in these plates overnight at 4° C. together with an antiphosphotyrosine antibody coupled with alkaline phosphatase (PY20:AP from Zymed). The (plates are washed again and the) binding of the antiphosphotyrosine antibody to the captured phosphorylated receptor is then demonstrated using a luminescent AP substrate (CDP-Star, ready to use, with Emerald II; Applied Biosystems). The luminescence is measured in a Packard Top Count Microplate Scintillation Counter. The difference between the signal of the positive control (stimulated with VEGF) and that of the negative control (not stimulated with VEGF) corresponds to VEGF-induced VEGF-R2 phosphorylation (=100%). The activity of the tested substances is calculated as percent inhibition of VEGF-induced. VEGF-R2 phosphorylation, wherein the concentration of substance that induces half the maximum inhibition is defined as the IC₅₀ (inhibitory dose for 50% inhibition). The NOVEL COMPOUNDS OF THE INVENTION here show an IC₅₀ in the range of 0.0003 to 20 μM, preferably between 0.001 and 10 μM.

In analogy, VEGF-R₁ inhibition can be shown as follows: The test is conducted using Flt-1 VEGF receptor tyrosine kinase. The detailed procedure is as follows: 30 μg/ml kinase solution (10 ng of the kinase domain of Flt-1, Shibuya et al., Oncogene 5, 519-24 (1990)) in 20 mM Tris-HCl pH 7.5, 3 mM manganese dichloride (MnCl₂), 3 mM magnesium chloride (MgCl₂), 10 mM sodium vanadate, 0.25 mg/ml polyethylenglycol (PEG) 20 000, 1 mM dithiothreitol and 3 μg/ml poly(Glu, Tyr) 4:1 (Sigma, Buchs, Switzerland), 8 μM [γ³³P]-ATP (0.2 μCi), 1% dimethyl sulfoxide, and 0 to 100 μM of the NOVEL COMPOUND OF THE INVENTION to be tested are incubated together for 10 min at room temperature. The reaction is then terminated by the addition of 10 μl 0.25 M ethylenediamine tetraacetate (EDTA) pH 7. Using a multichannel dispenser (LAB SYSTEMS, USA), an aliquot of 20 μl is applied to a PVDF (=polyvinyl difluoride) Immobilon P membrane (Millipore, USA), through a Millipore microtiter filter manifold and connected to a vacuum. Following complete elimination of the liquid, the membrane is washed 4 times successively in a bath containing 0.5% phosphoric acid (H₃PO₄) and once with ethanol, incubated for 10 min each while shaking, then mounted in a Hewlett Packard TopCount Manifold and the radioactivity measured after the addition of 10 μl Microscint® (β-scintillation counter liquid). IC₅₀ values are determined by linear regression analysis of the percentages of inhibition of each compound in three conditions (as a rule 0.01, 0.1 and 1 μmol). The IC₅₀ values that can be found with the NOVEL COMPOUNDS OF THE INVENTION are in the range of 0.01 to 100 μM, preferably in the range from 0.01 to 50 μM.

Flt-3 kinase inhibition is determined as follows: The baculovirus donor vector pFbacG01 (GIBCO) is used to generate a recombinant baculovirus expressing the amino acid region amino acids 563-993 of the cytoplasmic kinase domain of human Flt-3. The coding sequence for the cytoplasmic domain of Flt-3 is amplified by PCR from human c-DNA libraries (Clontech). The amplified DNA fragments and the pFbacG01 vector are made compatible for ligation by digestion with BamHI and HindIII. Ligation of these DNA fragments results in the baculovirus donor plasmid pFbacG01-Flt-3. The production of the viruses, the expression of proteins in Sf9 cells and the purification of the GST-fused proteins are performed as follows:

Production of virus: The baculovirus donor plasmid (pFbacG01-Flt-3) containing the Flt-3 kinase domain is transfected into the DH10Bac cell line (GIBCO) and the transfected cells are plated on selective agar plates. Colonies without insertion of the fusion sequence into the viral genome (carried by the bacteria) are blue. Single white colonies are picked and viral DNA (bacmid) is isolated from the bacteria by standard plasmid purification procedures. Sf9 or Sf21 cells (American Type Culture Collection) are then transfected in flasks with the viral DNA using Cellfectin reagent.

Protein expression in Sf9 cells: Virus containing media is collected from the transfected cell culture and used for infection to increase its titre. Virus containing media obtained after two rounds of infection is used for large-scale protein expression. For large-scale protein expression 100 cm² round tissue culture plates are seeded with 5×10⁷ cells/plate and infected with 1 mL of virus-containing media (approx. 5 MOIs). After 3 days the cells are scraped off the plate and centrifuged at 500 rpm for 5 min. Cell pellets from 10-20, 100 cm² plates are resuspended in 50 mL of ice-cold lysis buffer (25 mM Tris-HCl, pH 7.5, 2 mM EDTA-1% NP-40. 1 mM DTT, 1 mM PMSF). The cells are stirred on ice for 15 min and then centrifuged at 5000 rpms for 20 min.

Purification of GST-tagged protein: The centrifuged cell lysate is loaded onto a 2 mL glutathione-sepharose column (Pharmacia) and washed three times with 10 mL of 25 mM Tris-HCl, pH 7.5, 2 mM EDTA, 1 mM DTT, 200 mM NaCl. The GST-tagged protein is then eluted by 10 applications (1 mL each) of 25 mM Tris-HCl, pH 7.5, 10 mM reduced-glutathione, 100 mM NaCl, 1 mM DTT, 10% Glycerol and stored at −70° C.

Measurement of enzyme activity: Tyrosine protein kinase assays with purified GST-Flt-3 protein are carried out in a final volume of 30 μL containing 200-1800 ng of enzyme protein (depending on the specific activity), 20 mM Tris-HCl, pH 7.6, 3 mM MnCl₂, 3 mM MgCl₂, 1 mM DTT, 10 μM Na₃VO₄, 3 μg/mL poly(Glu, Tyr) 4:1, 1% DMSO, 8.0 μM ATP and 0.1 μCi [γ³³ P] ATP. The activity is assayed in the presence or absence of inhibitors, by measuring the incorporation of ³³P from [γ³³P] ATP into the poly(Glu,Tyr) substrate. The assay (30 μL) is carried out in 96-well plates at ambient temperature for 20 min and terminated by the addition of 20 μL of 125 mM EDTA. Subsequently, 40 μL of the reaction mixture is transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μL/well of Microscint™ (Packard). IC₅₀ values are calculated by linear regression analysis of the percentage inhibition of each compound in duplicate, at four concentrations (usually 0.01, 0.1, 1 and 10 μM). One unit of protein kinase activity is defined as 1 nmole of ³³P transferred from [γ₃P] ATP to the substrate protein per minute per mg of protein at 37° C. The NOVEL COMPOUNDS OF THE INVENTION here show IC₅₀ values in the range between 0.005 and 20 μM, preferably between 0.01 and 10 μM.

### Inhibition of Proliferation in Flt-3 Dependent Ba/F3 Cells:

The compound's potential to penetrate cell membranes and exert antiproliferative effects is determined in Ba/F3 cells dependent on mutated [ITD or D835Y; Gilliland and Griffin, Blood, Vol. 100, No. 5, 153242 (2002)] Flt-3 receptor kinases.

A modified protocol of the YO-PRO-1 assay in a 96-well format is based on the use of the wild-type IL-3-dependent hematopoietic cell line Ba/F3 (DSMZ, Braunschweig, Germany) and the mutant sub-lines ITD-Ba/F3 or D835Y-Ba/F3 [Weisberg et al., Cancer Cell 1 (5):433-43 (2000)] expressing constitutively activating Flt-3 kinases.

ITD-FLT3- or D835Y-FLT3-Ba/F3 cells are diluted in fresh medium to a final concentration of 3×10⁵ cells per ml and 50 μl aliquots seeded into 96-well plates (1.5×10⁴ cells per well). Subsequently, 50 μl 2× compound solutions were added and cells incubated for 48 h. The anti-proliferative and apoptotic activity of a compound is initially tested in triplicates at 10 μM, 1 μM and 0.1 μM concentration on both cell lines. Cells treated with DMSO alone (added to a final concentration of 0.1%) always serves as a control. In addition, a plate blank value is routinely determined in a well containing only 100 μl of medium and no cells.

To further profile a compound an ED₅₀ determination is done starting at 10 μM or 3 μM of the compound of interest. From those concentrations, stepwise nine dilutions are prepared reaching the final concentrations of 2 μM and 0.5 μM, respectively.

Activity of inhibitors is assessed by the YO-PRO-1 assay as previously described in [Idziorek et al., J. Immunol. Methods; 185:249-58 (1995)]. Briefly, after the treatment period of 48 h, a 25 μl aliquot of a solution containing 100 mM sodium citrate, pH 4.0, 134 mM sodium chloride and 12.5 μM YO-PRO-1 dye (YO-PRO-1 iodide, #Y3603, Molecular Probes) is directly added to the 100 μl medium in the wells of the 96-well plate. That results in a final dye concentration of 2.5 μM, the plate is then incubated for 10 min at ambient temperature in the dark. The uptake of the YO-PRO-1 dye into cells is assessed by a first measurement using a Cytofluor II 96-well plate reader (PerSeptive Biosystems) with the following settings: Excitation (nm) 485120 and Emission (nm) 530/25, Gain 75. After this first reading, 25 μl of lysis buffer consisting of 20 mM sodium citrate, pH 4.0, 26.8 mM sodium chloride, 0.4% NP40, 20 mM EDTA and 20 mM is added to each well. Cell lysis is completed within 60 min at room temperature and total amount of YO-PRO-1 bound to DNA is determined by a second measurement using the Cytofluor II 96-well reader with the identical setting as described above. Using this assay, the NOVEL COMPOUNDS OF THE INVENTION exhibit ED₅₀ values for both mutant sub-lines in range of from 0.1 nM to 1 μM, especially from 0.1 nM to 100 nM.

### Tek Kinase Inhibition can be Performed as Follows:

The baculovirus donor vector pFbacG01 is used to generate a recombinant baculovirus that expressed the amino acid region amino acids 773-1124 of the cytoplasmic kinase domain of human Tek, N-terminally fused to GST. Tek is recloned into the pFbacG01 transfer vector by EcoRI excision and ligation into EcoRI digested pFbacG01 (FBG-Tie2/Tek). The production of the viruses, the expression of proteins in Sf9 cells and the purification of the GST-fused proteins are performed as following:

Production of virus: Transfer vectors containing the kinase domain are transfected into the DH10Bac cell line (GIBCO) and the transfected cells are plated on selective agar plates. Colonies without insertion of the fusion sequence into the viral genome (carried by the bacteria) are blue. Single white colonies are picked and viral DNA (bacmid) is isolated from the bacteria by standard plasmid purification procedures. Sf9 cells or Sf21 cells (American Type Culture Collection) are then transfected in 25 cm² flasks with the viral DNA using Cellfectin reagent.

Protein expression in Sf9 cells: Virus containing media is collected from the transfected cell culture and used for infection to increase its titer. Virus containing media obtained after two rounds of infection is used for large-scale protein expression. For large-scale protein expression 100 cm² round tissue culture plates are seeded with 5×10⁷ cells/plate and infected with 1 mL of virus-containing media (approx. 5 MOIs). After 3 days the cell is are scraped off the plate and centrifuged at 500 rpm for 5 min. Cell pellets from 10-20, 100 cm² plates are resuspended in 50 mL of ice-cold lysis buffer (25 mM Tris-HCl, pH 7.5, 2 mM EDTA, 1% NP-40, 1 mM DTT, 1 mM PMSF). The cells are stirred on ice for 15 min and then centrifuged at 5000 rpms for 20 min.

Purification of GST-tagged protein: The centrifuged cell lysate is loaded onto a 2 mL glutathione-sepharose column (Pharmacia) and washed three times with 10 mL of 25 mM Tris-HCl, pH 7.5, 2 mM EDTA, 1 mM DTT, 200 mM NaCl. The GST-tagged Tek is eluted by 10 applications (1 mL each) of 25 mM Tris-HCl, pH 7.5, 10 mM reduced-glutathione, 100 mM NaCl, 1 mM DTT, 10% Glycerol and stored at −70° C.

Kinase assay: Tyrosine protein kinase assays with purified GST-Tek protein are carried out in a final volume of 30 μL containing 15 mg/ml GST-Tek, 20 mM Tris-HCl, pH 7.5, 3 mM MnCl₂, 3 mM MgCl₂, 1 mM DTT, 10 μM Na₃VO₄, 3.0 μg/mL poly(Glu,Tyr) 4:1, PEG 0.25 mM, 1% DMSO, 8.0 μM ATP, [γ³³P] ATP 0.1 μCi). The activity is assayed in the presence or absence of inhibitors, by measuring the incorporation of ³³P from [γ³³P] ATP into poly(Glu, Tyr) 4:1. The assay (30 μL) is carried out in 96-well plates at ambient temperature for 10 min and terminated by the addition of 20 μL of 125 mM EDTA. Subsequently, 40 μL of the reaction mixture are transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μL 0.5% H₃PO₄. Membranes are removed and washed 4× on a shaker with 1.0% H₃PO₄, once with ethanol. Membranes are counted after driving at ambient temperature, mounting in Packard TopCount 96-well frame, and addition of 10 μL/well of Microscint™ (Packard). IC₅₀ values are calculated by linear regression analysis of the percentage inhibition of each compound in duplicate, at four concentrations (usually 0.01, 0.1, 1 and 10 μM). One unit of protein kinase activity is defined as 1 nmole of ³³P transferred from [γ³³P] ATP to the substrate protein per minute per mg of protein at 37° C. The NOVEL COMPOUNDS OF THE INVENTION here show IC₅₀ values in the range between 0.001 and 5 μM, especially between 0.01 and 0.2 μM. Bcr-Abl inhibition can be determined by a capture ELISA as follows: The murine myeloid progenitor cell line 32Dcl3 transfected with the p210 Bcr-Abl expression vector pGDp210Bcr/Abl (32D-bcr/abl) is obtained from J Griffin (Bazzoni et al., J. Clin Invest. 98, 521-8 (1996); Zhao et al., Blood 90, 4687-9 (1997)). The cells express the fusion bcr-abl protein with a constitutively active abl kinase and proliferate growth factor-independent. The cells are expanded in RPMI 1640 (AMIMED; cat# 1-41F01), 10% fetal calf serum, 2 mM glutamine (Gibco) (“complete medium”), and a working stock is prepared by freezing aliquots of 2×10⁶ cells per vial in freezing medium (95% fetal calf serum, 5% dimethylsulfoxide (SIGMA, D-2650). After thawing, the cells are used during maximally 10-12 passages for the experiments. The antibody anti-abl SH3 domain cat. # 06466 from Upstate Biotechnology is used for the ELISA. For detection of bcr-abl phosphorylation, the anti-phosphotyrosine antibody Ab PY20, labelled with alkaline phosphatase (PY10(AP)) from ZYMED (cat. # 03-7722) is used. As comparison and reference compound, (N-{5-[4-(4-methyl-piperazino-methyl)-benzoylamido]-2-methylphenyl}-4-(3-pyridyl)-2-pyrimidine-amine, in the form of the methane sulfonate (monomesylate) salt (ST1571) (marketed as Gleevec® or Glivec®, Novartis), is used. A stock solution of 10 mM is prepared in DMSO and stored at −20° C. For the cellular assays, the stock solution is diluted in complete medium in two steps (1:100 and 1:10) to yield a starting concentration of 10 μM followed by preparation of serial threefold dilutions in complete medium. No solubility problems are encountered using this procedure. The test NOVEL COMPOUNDS OF THE INVENTION are treated analogously. For the assay, 200,000 32D-bcr/abl cells in 50 μl are seeded per well in 96 well round bottom tissue culture plates. 50 μl per well of serial threefold dilutions of the test compound are added to the cells in triplicates. The final concentration of the test compound range e.g. from 5 μM down to 0.01 μM. Untreated cells are used as control. The compound is incubated together with the cells for 90 min at 37° C., 5% CO₂, followed by centrifugation of the tissue culture plates at 1300 rpm (Beckman GPR centrifuge) and removal of the supernatants by careful aspiration taking care not to remove any of the pelleted cells. The cell pellets are lysed by addition of 150 μl lysis buffer (50 mM Tris/HCl, pH 7.4, 150 mM sodium chloride, 5 mM EDTA, 1 mM EGTA, 1% NP-40 (non-ionic detergent, Roche Diagnostics GmbH, Mannheim, Germany), 2 mM sodium ortho-vanadate, 1 mM phenylmethyl sulfonylfluoride, 50 μg/ml aprotinin and 80 μg/ml leupeptin) and either used immediately for the ELISA or stored frozen at −20° C. until usage. The anti-abl SH3-domain antibody is coated at 200 ng in 50 μl PBS per well to black ELISA plates (Packard HTRF-96 black plates; 6005207) overnight at 4° C. After washing 3× with 200 μl/well PBS containing 0.05° % Tween 20 (PBST) and 0.5% TopBlock (Juro, Cat. #TB 232010), residual protein binding sites are blocked with 200 μl/well PBST, 3% TopBlock for 4 h at room temperature, followed by incubation with 50 μl lysates of untreated or test compound-treated cells (20 μg total protein per well) for 3-4 h at 4° C. After 3× washing, 50 μl/well PY20(AP) (Zymed) diluted to 0.5 μg/ml in blocking buffer is added and incubated overnight (4 IC). For all incubation steps, the plates are covered with plate sealers (Costar, cat. #3095). Finally, the plates are washed another three times with washing buffer and once with deionized water before addition of 90 μl/well of the AP substrate CPDStar RTU with Emerald II. The plates now sealed with Packard Top Seal™-A plate sealers (cat. #6005185) are incubated for 45 min at room temperature in the dark and luminescence is quantified by measuring counts per second (CPS) with a Packard Top Count Microplate Scintillation Counter (Top Count). For the final optimized version of the ELISA, 50 μl of the lysates of the cells grown, treated and lysed in 96 well tissue culture plates, are transferred directly from these plates to the ELISA plates that are precoated with 50 ng/well of the rabbit polyclonal ant-abl-SH3 domain AB 06466 from Upstate. The concentration of the anti-phosphotyrosine AB PY20 (AP) can be reduced to 0.2 μg/ml. Washing, blocking and incubation with the luminescent substrate are as above. The quantification is achieved as follows: The difference between the ELISA readout (CPS) obtained for with the lysates of the untreated 32D-bcr/abl cells and the readout for the assay background (all components, but without cell lysate) is calculated and taken as 100% reflecting the constitutively phosphorylated bcr-abl protein present in these cells. The activity of the compound in the bcr-abl kinase activity is expressed as percent reduction of the bcr-abl phosphorylation. The values for the IC₅₀ are determined from the dose response curves by graphical inter- or extrapolation. The NOVEL COMPOUNDS OF THE INVENTION here preferably show IC₅₀ values in the range from 20 nM to 200 μM.

The NOVEL COMPOUNDS OF THE INVENTION also inhibit protein tyrosine kinases that are involved in the signal transmission mediated by trophic factors, for example kinases of the src kinase family, such as especially the c-Src kinase, members of the platelet-derived growth factor (PDGF) receptor tyrosine kinase family, for example PDGF-R, c-Kit, VEGF-R and/or FGF-R; all of which play a part in growth regulation and transformation in animal, especially mammal cells, including human cells. An appropriate assay is described in Andrejauskas-Buchdunger et al., Cancer Res. 52, 5353-8 (1992).

The NOVEL COMPOUNDS OF THE INVENTION can therefore be used in the treatment of protein kinase dependent diseases. Protein kinase dependent diseases are especially proliferative diseases, preferably benign or especially malignant tumours (for example carcinoma of the kidneys, liver, adrenal glands, bladder, breast, stomach, ovaries, colon, rectum, prostate, pancreas, lungs, vagina or thyroid, sarcoma, glioblastomas and numerous tumours of the neck and head, as well as leukemias). They are able to bring about the regression of tumours and to prevent the formation of tumour metastases and the growth of (also micro)metastases. In addition they can be used in epidermal hyperproliferation (e.g. psoriasis), in prostate hyperplasia, and in the treatment of neoplasias, especially of epithelial character, for example mammary carcinoma. It is also possible to use the NOVEL COMPOUNDS OF THE INVENTION in the treatment of diseases of the immune system insofar as several or, especially, individual protein tyrosine kinases are involved; furthermore, the NOVEL COMPOUNDS OF THE INVENTION can be used also in the treatment of diseases of the central or peripheral nervous system where signal transmission by at least one protein tyrosine kinase, especially selected from those mentioned specifically, is involved.

The p21ras oncogene is a major contributor to the development and progression of human solid cancers and is mutated in 30% of all human cancers. The endogenous GTPase activity, if alleviated in ras mutated cancer cells, mediates constitutive growth signals to down-stream effectors such as raf kinase. Inhibiting the raf kinase signalling pathway can therefore be used for inhibiting the effect of active ras. The NOVEL COMPOUNDS OF THE INVENTION useful as ras inhibitors are thus especially appropriate for the therapy of diseases related to ras overexpression or overactivity.

Vascular endothelial growth factor receptor-2 (VEGF-R2; KDR) is selectively expressed on the primary vascular endothelium and is essential for normal vascular development. In order to grow beyond minimal size, tumours must generate new vascular supply. Angiogenesis, or the sprouting of new blood vessels, is a central process in the growth of solid tumours. For many [symbol] the extent of vascularization of a tumour is a negative prognostic indicator signifying aggressive disease and increased potential for metastasis. Recent efforts to understand the molecular basis of tumour-associated angiogenesis have identified several potential therapeutic targets, including the receptor tyrosine kinases for the angiogenic factor vascular endothelial growth factor (VEGF) (see Zeng et al., J. Biol. Chem. 276(35); 32714-32719 (2001)). The NOVEL COMPOUNDS OF THE INVENTION useful as KDR inhibitors are thus especially appropriate for the therapy of diseases related to VEGF receptor tyrosine kinase overexpression. Among these diseases, especially retinopathies, age-related macula degeneration, psoriasis, haemangioblastoma, haemangioma, arteriosclerosis, inflammatory diseases, such as rheumatoid or rheumatic inflammatory diseases, especially arthritis, such as rheumatoid arthritis, or other chronic inflammatory disorders, such as chronic asthma, arterial or post-transplantational atherosclerosis, endometriosis, and especially neoplastic diseases, for example so-called solid tumours (especially cancers of the gastrointestinal tract, the pancreas, breast, stomach, cervix, bladder, kidney, prostate, ovaries, endometrium, lung, brain, melanoma, Kaposi's sarcoma, squamous cell carcinoma of head and neck, malignant pleural mesotherioma, lymphoma or multiple myeloma) and liquid tumours (e.g. leukemias) are especially important.

Flt-3 (FMD-like tyrosine kinase) is especially expressed in hematopoietic progenitor cells and in progenitors of the lymphoid and myeloid series. Aberrant expression of the Flt-3 gene has been documented in both adult and childhood leukemias including AML (acute myelogenous leukemia), AML with trilineage myelodysplasia (AML/TMDS), ALL (acute lymphoblastic leukemia), CML (chronic myelogenous leukemia) and myelodysplastic syndrome (MDS), which are therefore the preferred diseases to be treated with the NOVEL COMPOUNDS OF THE INVENTION. Activating mutations in Flt-3 have been found in approximately 25 to 30% of patients with AML. Thus there is accumulating evidence for the role of Flt-3 in human leukemias, and the NOVEL COMPOUNDS OF THE INVENTION useful as Flt-3 inhibitors are especially of use in the therapy of this type of diseases (see Tse et al., Leukemia 15(7), 1001-1010 (2001); Tomoki et al., Cancer Chemother. Pharmacol. 48 (Suppl. 1), S27-S30 (2001); Birkenkamp et al., Leukemia 15(12), 1923-1921 (2001); Kelly et al., Neoplasia 99(1), 310-318 (2002)).

In chronic myelogeous leukemia (CML), a reciprocally balanced chromosomal translocation in hematopoietic stem cells (HSCs) produces the BCR-ABL hybrid gene. The latter encodes the oncogenic Bcr-Abl fusion protein. Whereas ABL encodes a tightly regulated protein tyrosine kinase, which plays a fundamental role in regulating cell proliferation adherence and apoptosis, the BCR-ABL fusion gene encodes as constitutively activated kinase, which transforms HSCs to produce a phenotype exhibiting deregulated clonal proliferation, reduced capacity to adhere to the bone marrow stroma and a reduces apoptotic response to mutagenic stimuli, which enable it to accumulate progressively more malignant transformations. The resulting granulocytes fail to develop into mature lymphocytes and are released into the circulation, leading to a deficiency in the mature cells and increased susceptibility to infection. ATP-competitive inhibitors of Bcr-Abl have been described which prevent the kinase from activating mitogenic and anti-apoptotic pathways (e.g. P-3 kinase and STAT5), leading to the death of the BCR-ABL phenotype cells and thereby providing an effective therapy against CML. The NOVEL COMPOUNDS OF THE INVENTION useful as Bcr-Abl inhibitors are thus especially appropriate for the therapy of diseases related to its overexpression, especially leukemias, such as leukemias, e.g. CML or ALL.

The NOVEL COMPOUNDS OF THE INVENTION, in view of their activity as PDGF receptor inhibitors, are also especially appropriate in the treatment of proliferative diseases, especially small lung cancer, atherosclerosis, thrombosis, psoriasis, scleroderma or fibrosis.

There are also experiments to demonstrate the antitumour activity of the compounds of the present invention in vivo: The in vivo antitumour activity is tested, for example, using breast carcinoma cell lines, such as the human estrogen dependent breast carcinoma MCF-7 (ATCC: HTB22) or ZR-75-1 (ATCC: CRL1500), or the estrogen-independent breast carcinomas MDA-MB468 (ATCC: HTB132) or MDA-MB231 (ATCC: HTB26); colon carcinoma cell lines, such as the colon-carcinoma Colo 205 (ATCC: CCL222); glioblastoma cell lines, such as the glioblastomas U-87MG (ATCC: HTB14) or U-373MG (ATCC: HTB17); lung carcinoma cell lines, such as the “small cell lung carcinomas” NCI-H69 (ATCC: HTB119) or NCI-H209 (ATCC: HTB172), or the lung carcinoma NCI-H596 (ATCC: HTB178); skin tumour cell lines, such as the melanomas Hs294T (ATCC: HTB140) or A375 (ATCC: CRL1619); tumour cell lines from the genitourinary systems, such as the ovarial carcinoma NIH-Ovcar3 (ATCC: HTB161), as well as the prostate carzinomas DU145 (ATCC: HTB81) or PC-3 (ATCC: CRL1435), or the bladder carcinoma T24 (ATCC: HTB4); epithelial carcinomas, such as the epithelial carcinoma KB31; or (especially with regard to leukemias) K562 cells (American Type Culture Collection, Mannassas, Va.) or human CFU-G cells (CFU-G stands for granulocyte colony forming unit, and it represents an early but committed granulocyte forming precursor cell that circulates in the blood stream or bone marrow) each of which is transplanted into female or male Balb/c nude mice. Other cell lines include leukemic cell lines such as K-562, SUPB15, MEG01, Ku812F, MOLM-13, BaF3, CEM/0, JURKAT/0 or U87MG.

Tumours are obtained after subcutaneous injection of the respective cells (minimum 2×10⁶ cells in 100 ml phosphate buffered physiological saline) into the carrier mice (e.g. 4-8 mice per cell line). The resulting tumours are passed serially through at least three subsequent transplantations before treatment is started. Tumour fragments (about 25 mg each) are injected s.c. into the left flank of the animals using a 13-gauge Trocar needle under Forene narcosis (Abbott, Switzerland) for implantation. Mice transplanted with estrogen-dependent tumour are, in addition, supplied with an estrogen pellet (1.0 cm of a tube with a quality appropriate for medical purposes, Dow Chemicals, with 5 mg estradiole, Sigma). The treatment is started routinely (that is at low or intermediate tumour burden), as soon as the tumour has reached an average size of 100 mm³. Tumour growth is determined once, twice or thrice weekly (depending on tumour growth of the cell line) and 24 h after the last treatment by measurement of the perpendicular diameter. In case of tumours, tumour volumes are determined according to the formula L×D×p/6 (see Evans, B. D., Smith, I. E., Shorthouse, A. J. and Millar, J. J., Brit. J. Cancer, 45: 466-468, 1982). The antitumour activity is expressed as T/C % (average increase of the tumour volume of treated animals divided by the average increase of tumour volume in control animals multiplied by 100). Tumour-regression (%) represents the smallest mean tumour volume compared to the mean tumour volume at the beginning of the treatment. Each animal in which the tumour reaches a diameter of more than 1.5 to 2 cm³ is sacrificed. Leukemia burden is assessed by examining both peripheral white blood count and weight of spleen and thymus in animals tumoured with leukemia cell lines.

An exemplary (though not limiting) schedule for administration of a compound of the present invention, or a salt thereof, is daily administration, with preferably 1 to 3 daily dosages for a longer time, possibly until the disease is cured or, if only palliative treatment is achieved, for as long as required; alternatively, treatment e.g. for 5 days, and/or administration at days 1, 4 and 9, with eventual repetition after a certain time without treatment is possible. Alternatively, treatment several times a day (e.g. 2 to 5 times) or treatment by continuous administration (e.g. infusion), e.g. at the time points indicated in the last sentence, are possible. Generally, administration is orally or parenterally, preferably orally. The test compounds are preferably diluted in water or in sterile 0.9% saline.

All human tumour cell lines are obtained from the American Type Culture Collection (ATCC, Rockville, Md., USA) if not indicated otherwise and are cultivated in the suggested media with the corresponding additives (ATCC culture conditions), if not mentioned otherwise. The c-sis- and v-sis-transformed BALB/c 3T3 cells are obtained from Dr. C. Stiles (Dana Farber Cancer Institute, Boston, Mass., USA). They are cultured in “Dulbecco's modified Eagle's medium” (DMEM), that is supplemented with 10% calf serum and Hygromycin B in a concentration of 0.2 mg/ml or G418 in a concentration of 0.5 mg/ml. BALB/c AMuLV A.6R.1 cells (ATCC) are kept in DMEM, supplemented with 10% fetal calf serum.

The pharmacological activity of a compound of the present invention may, for example, be demonstrated in a clinical study or in a test procedure as essentially described hereinafter.

Suitable clinical studies are, for example, open label non-randomized, dose escalation studies in patients with one of the tumour diseases mentioned above. The beneficial effects on proliferative diseases can be determined directly through the results of these studies or by changes in the study design which are known as such to a person skilled in the art. The efficacy of the treatment can be determined in such studies, e.g., in case of tumours after 18 or 24 weeks by radiologic evaluation of the tumours every 6 weeks, in case of a leukaemia e.g. by determination of the count of aberrant white blood cells, and by staining mononuclear cells and/or by means of determining minimum residual disease (MRD) e.g. by FACS-LPC MRD or PCR.

Alternatively, a placebo-controlled, double blind study can be used in order to prove the benefits of the compounds of the present invention.

The diaryl urea derivatives of formula I can be prepared as described in WO 03/099771. The NOVEL COMPOUNDS OF THE INVENTION are preferably prepared as described hereinbelow under “Examples”.

### PREFERRED EMBODIMENTS ACCORDING TO THE INVENTION

In the following preferred embodiments, general expression can be replaced by the corresponding more specific definitions provided above and below, thus yielding stronger preferred embodiments of the invention.

In a preferred embodiment the invention relates to the use of dairy urea derivatives for the manufacture of pharmaceutical compositions for use in the treatment of RET dependent diseases, wherein the diaryl urea derivative is a compound of the formula I*

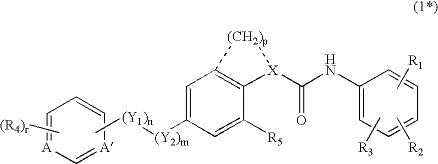

wherein A, A′, n, m, p, r, X, Y₁, Y₂ and R₁-R₆ have the meanings as defined above for a compound of formula I;
or a tautomer thereof;
or pharmaceutically acceptable salts thereof.

In another preferred embodiment the invention relates the use of diaryl urea derivatives for the manufacture of pharmaceutical compositions for use in the treatment of RET dependent diseases, wherein the diaryl urea derivative is a compound of the formula I, wherein

A is CH, N or N→O and A′ is N or N→O, with the proviso that not more than one of A and A′ can be N→O;
n is 1 or 2;
m is 0, 1 or 2;
p is 0, 2 or 3;
r is 1 to 5;
X is NR if p is 0, wherein R is hydrogen or an organic moiety, or if p is 2 or 3, X is nitrogen which together with (CH₂)ₚ and the bonds represented in dotted (interrupted) lines (including the atoms to which they are bound) forms a ring,
with the proviso that if X is NH, each of R₄, independently of the others if r>1, is a moiety as defined above under formula I but not bound to the rest of formula I via a —C(═O)—, —C(NR)— or —S(O₂)— bridge,
or
X is CHK wherein K is lower alkyl or hydrogen and p is zero,
with the proviso that the bonds represented in dotted lines, if p is zero, are absent;

### Y₁ is O, S or CH₂;

### Y₂ is O, S or NH;

with the proviso that (Y₁)ₙ—(Y₂)ₘ does not include C—C, S—S, NH—C, NH—S or S—O groups;
each of R₁, R₂, R₃ and R₅, independently of the others, is hydrogen or an inorganic or organic moiety or any two of R₁, R₂ and R₃ together form a lower alkylene-dioxy bridge bound via the oxygen atoms, and the remaining one of these moieties is hydrogen or an inorganic or organic moiety, with the proviso that if G is not present and Z is a radical of the formula Ia, R₁, R₂ and R₃ cannot all be hydrogen and with the further proviso that if one of R₁, R₂ and R₃ is halo or lower alkyl-sulfonyl, the other two cannot both be hydrogen;

R₄ is an inorganic or organic moiety, with the proviso that if n is l, m is 0, p is 0, r is 1, X is NH, Y₁ is O, G is not present and Z is a radical of the formula Ia, R₄, together with the benzene ring containing A and A′, does not form methylpyridinyl, 2-hydroxy-pyridin-4-yl or 1-H-2-oxo-1,2-dihydropyridin-4-yl; and

G and Z have the meanings given above under formula I;
or a tautomer thereof;
or pharmaceutically acceptable salts thereof.

In further preferred embodiment the invention relates the use of diaryl urea derivatives for the manufacture of pharmaceutical compositions for use in the treatment of RET dependent diseases, wherein the diaryl urea derivative is a compound of the formula I*, wherein

A is CH, N or N→O and A′ is N or N→O, with the proviso that not more than one of A and A′

### can be N→O;

n is 1;
m is 0;
p is 0, 2 or 3;
r is 1;
X is NR if p is 0, wherein R is hydrogen or lower alkyl, or if p is 2 or 3, X is nitrogen which together with (CH₂)ₚ and the bonds represented in dotted (interrupted) lines (including the atoms to which they are bound) forms a ring, or
X is CH₂ and p is zero,
with the proviso that the bonds represented in dotted lines, if p is zero, are absent;

### Y₁ is O or CH₂;

each of R₁, R₂ and R₃ independently of the others, is hydrogen, lower alkyl, halo, especially bromo or chloro, halo-lower alkyl, especially trifluoromethyl, lower alkoxy, especially methoxy, halo-lower alkoxy, especially 2,2,2-trifluoroethoxy, phenyl, piperidyl, especially piperidin-1-yl, piperazinyl, especially piperazin-1-yl, morpholinyl, especially morpholine, thiomorpholinyl, especially thiomorpholino, or any two of them together form a lower alkylene-dioxy bridge [symbol] the oxygen atoms, and the remaining one of these moieties is hydrogen or one of the moieties mentioned, with the proviso that R₁, R₂ and R₃ cannot all be hydrogen and with the further proviso that one if R₁, R₂ and R₃ is halo, the other two cannot both be hydrogen;
R₄ is lower alkoxy, especially methoxy, lower alkanoylamino, especially acetylamino, hydroxyphenylamino, especially p-hydroxyphenylamino, amino-lower alkyl-oxyphenyl-amino, especially 4-[(2-aminoethyl)-oxyphenyl]-amino, sulfamoylphenylamino, especially 4-sulfamoylphenylamino, carbamoylphenylamino, especially 4-carbamoylphenylamino, [N-(hydroxy-lower alkyl)-carbamoyl]-phenylamino, especially [N-(2-hydroxyethyl)-carbamoyl]-phenylamino, or halo, especially chloro; and
R₅ is hydrogen, lower alkyl or halo, especially hydrogen;
or a tautomer thereof;
or pharmaceutically acceptable salts thereof.

In further especially preferred embodiment the invention relates the use of diaryl urea derivatives for the manufacture of pharmaceutical compositions for use in the treatment of RET dependent diseases, wherein the diaryl urea derivative is a compound of the formula I, wherein

G is either not present, lower alkylene, especially methylene or ethylene, or C₃-C₅cycloalkylene, especially cyclopropylene, and Z is a radical of the formula Ia, or
G is not present and Z is a radical of the formula Ib;

### A is CH or N and A′ is N or N→O;

n is 1;
m is 0 or 1;
p is 0, 2 or 3;
r is 0 or 1;
X is NR if p is 0, wherein R is hydrogen or lower alkyl, or if p is 2 or 3, X is nitrogen which together with (CH₂)ₚ and the bonds represented in dotted (interrupted) lines (including the atoms to which they are bound) forms a ring, or
X is CHK wherein K is hydrogen and p is zero,
with the proviso that the bonds represented in dotted lines, if p is zero, are absent;

### Y₁ is O, S or CH₂;

### Y₂ is O;

with the proviso that (Y₁)ₙ—(Y₂)ₘ does not include O—, or —S—O groups;
each of R₁, R₂, and R₃, independently of the other is hydrogen, lower alkyl especially methyl, ethyl, n-propyl, isopropyl or tert-butyl, lower alkenyl, especially isopropenyl, hydroxy-lower alkyl, especially hydroxy-propyl, lower-alkoxy, especially methoxy, halo, especially chloro or bromo, halo-lower alkyl, especially trifluoromethyl, halo-lower alkoxy, especially trifluoromethoxy or trifluoroethoxy, amino-lower alkyl, especially aminomethyl, amino-lower alkoxy, especially aminoethoxy, di-lower alkyl-amino, especially diethylamino, hydroxy-lower alkyl-amino, especially hydroxy-propylamino, bis-(lower alkoxy-lower alkyl)-amino, especially bis-(2-methoxy-ethyl)-amino, di-lower alkyl-amino-lower alkyl, especially dimethylaminomethyl, phenyl, morpholinyl, especially morpholin-4-yl, piperidyl, especially piperidin-1-yl, piperidyl-lower alkyl, especially piperidin-1-ylmethyl, lower alkyl-piperazinyl, especially 4-methyl-piperazin-1-yl or 4-ethyl-piperazin-1-yl, lower alkyl-piperazinyl-lower alkyl, especially 4-methyl-piperazin-1-ylmethyl or 4-ethyl-piperazin-1-ylmethyl, pyridyl, especially pyridin-2-yl, or lower alkyl-imidazolyl, especially 2- or 4-methyl-imidazol-1-yl; if r is 1, R₄ is lower alkyl, especially methyl, ethyl or isopropyl, hydroxy, aminocarbonyl, lower alkyl-carbonyl, especially methylcarbonyl, cyclohexyl, halo, especially chloro or fluoro, halo-lower alkyl, especially trifluoromethyl, lower alkoxy, especially methoxy, amino, lower alkylamino, especially methylamino, ethylamino, isopropylamino or tert-butylamino, di-lower alkylamino, especially dimethylamino, lower alkenyl-amino, especially prop-2-enylamino or but-3-enylamino, lower alkyl-carbonyl-amino, especially methylcarbonylamino, cyano, azido, hydroxy-phenyl-amino, especially 3- or 4-hydroxy-phenyl-amino, mono or tri-lower alkoxy-phenyl-amino, especially methoxy-phenyl-amino or trimethoxy-phenyl-amino, lower alkoxy-halo-phenyl-amino, especially methoxy-fluoro-phenyl-amino, phenyl-lower alkylamino, especially benzylamino, (mono or di-lower alkoxy)-phenyl-lower alkylamino, especially methoxy-benzylamino or dimethoxy-benzylamino, aminosulfonyl-phenyl-lower alkylamino, especially aminosulfonyl-benzylamino, amino-lower alkoxy-phenyl-amino, especially aminoethoxy-phenyl-amino, lower alkyl-amino-sulfonyl-lower alkyl-phenylamino, especially methylamino-sulfonylmethyl-phenylamino, lower alkyl-piperazinyl-lower alkylamino, especially 4-methylpiperazin-1-yl-propylamino, morpholinyl-lower alkylamino, especially morpholin-4-yl-propylamino, lower alkyl-piperidyl-amino, especially 1-methyl-piperidin-4-ylamino, tetrazolyl, especially 1H-tetrazol-5-yl, lower alkyl-tetrazolyl, especially lower alkyl-tetrazol-5-yl such as 1-methyl-1H-tetrazol-5-yl or 2-methyl-2H-tetrazol-5-yl, or (di-lower alkyl)-amino-lower alkyl-tetrazolyl, especially (di-lower alkyl)-amino-lower alkyl-tetrazol-5-yl such as 2-(3-dimethylaminopropyl)-2H-tetrazol-5-yl; and
R₅ is most preferably hydrogen, or lower alkyl, especially methyl, or halo, especially chloro;
or a tautomer thereof;
or pharmaceutically acceptable salts thereof.

In another especially preferred embodiment the invention relates the use of diaryl urea derivatives for the manufacture of pharmaceutical compositions for use in the treatment of RET dependent diseases, wherein the diaryl urea derivative is a compound of the formula I, wherein

A and A′ are both N, n is 1, m is 0, p is 0 or 2, r is 1, X is NH if p is 0, or if p is 2, X is nitrogen which together with (CH₂)₂ and the bonds represented in dotted (interrupted) lines (including the atoms to which they are bound) forms a ring, Y₁ is O, G is not present, Z is a radical of the formula Ia, at least one of R₁, R₂ and R₃ is a basic organic moiety, R₄ is amino
or lower alkylamino and R₅ is hydrogen;
or a tautomer thereof;
or pharmaceutically acceptable salts thereof.

In another preferred embodiment the invention relates the use of diaryl urea derivatives for the manufacture of pharmaceutical compositions for use in the treatment of RET dependent diseases, wherein the diaryl urea derivative is a compound of the formula I*, wherein

A is CH, N or N→O and A′ is N or N→O, with the proviso that not more than one of A and A′ can be N→O;
n is 1;
m is 0;
p is 0, 2 or 3;
r is 0, 1 or 2;
X is NR if p is 0, wherein R is hydrogen or lower alkyl, or if p is 2 or 3, X is nitrogen which together with (CH₂)ₚ and the bonds represented in dotted (interrupted) lines (including the atoms to which they are bound) forms a ring, or
X is CH₂ and p is zero,
with the proviso that the bonds represented in dotted lines, if p is zero, are absent;

### Y₁ is O or CH₂;

each of R₁, R₂ and R₃ independently of the others, is hydrogen, lower alkyl, halo, especially bromo or chloro, halo-lower alkyl, especially trifluoromethyl, lower alkoxy, especially methoxy, halo-lower alkoxy, especially 2,2,2-trifluoroethoxy, phenyl, piperidyl, especially piperidin-1-yl, piperazinyl, especially piperazin-1-yl, morpholinyl, especially morpholine, thiomorpholinyl, especially thiomorpholino, or any two of them together form a lower alkylene-dioxy bridge bound via the oxygen atoms, and the remaining-one of these moieties is hydrogen or one of the moieties mentioned;
if r is not zero, R₄ is lower alkyl, especially methyl or ethyl, lower alkoxy, especially methoxy, lower alkanoylamino, especially acetylamino, hydroxyphenylamino, especially p-hydroxyphenylamino, amino-lower alkyl-oxyphenyl-amino, especially 4-[(2-aminoethyl)-oxyphenyl]-amino, sulfamoylphenylamino, especially 4-sulfamoylphenylamino, carbamoylphenylamino, especially 4-carbamoylphenylamino, [N-(hydroxy-lower alkyl)-carbamoyl]-phenylamino, especially [N-(2-hydroxyethyl)-carbamoyl]-phenylamino, halo, especially chloro, or hydroxyl; and
R₅ is hydrogen, lower alkyl or halo, especially hydrogen;
or a tautomer thereof;
or pharmaceutically acceptable salts thereof.

In another especially preferred embodiment the invention relates the use of diaryl urea derivatives for the manufacture of pharmaceutical compositions for use in the treatment of RET dependent diseases, wherein the diaryl urea derivative is a compound of the formula I, wherein

G is either not present, lower alkylene, especially methylene or ethylene, or C₃-C₅cycloalkylene, especially cyclopropylene, and Z is a radical of the formula Ia, or
G is not present and Z is a radical of the formula Ib;

### A is CH or N and A′ is N or N→O;

n is 1;
m is 0 or 1;
p is 0, 2 or 3;
r is 1;
X is NR if p is 0, wherein R is hydrogen or lower alkyl, or if p is 2 or 3, X is nitrogen, which together with (CH₂)ₚ and the bonds represented in dotted (interrupted) lines (including the atoms to which they are bound) forms a ring, or
X is CHK wherein K is hydrogen and p is zero,
with the proviso that the bonds represented in dotted lines, if p is zero, are absent;

### Y₁ is O, S or CH₂;

### Y₂ is O;

with the proviso that (Y₁)ₙ—(Y₂)ₘ does not include O—O, or S—O groups;
each of R₁, R₂ and R₃, independently of the others, is hydrogen, lower alkyl, especially methyl, ethyl, n-propyl, isopropyl or tert-butyl, lower alkenyl, especially isopropenyl, hydroxy-lower alkyl, especially hydroxy-propyl, lower alkoxy, especially methoxy, halo, especially chloro or bromo, halo-lower alkyl, especially trifluromethyl, halo-lower alkoxy, especially trifluoromethoxy or trifluoroethoxy, amino-lower alkyl, especially aminomethyl, amino-lower alkoxy, especially aminoethoxy, di-lower alkyl-amino, especially diethylamino, hydroxy-lower alkyl-amino, especially hydroxy-propylamino, bis-(lower alkoxy-lower alkyl)-amino, especially bis-(2-methoxy-ethyl)-amino, di-lower alkyl-amino-lower alkyl, especially dimethylaminomethyl, phenyl, morpholinyl, especially morpholin-4-yl, piperidyl, especially piperidin-1-yl, piperidyl-lower alkyl, especially piperidin-1-ylmethyl, lower alkyl-piperazinyl, especially 4-methyl-piperazin-1-yl or 4-ethyl-piperazin-1-yl, lower alkyl-piperazinyl-lower alkyl, especially 4-methyl-piperazin-1-ylmethyl or 4-ethyl-piperazin-1-ylmethyl, pyridyl, especially pyridin-2-yl, or lower alkyl-imidazolyl, especially 2- or 4-methyl-imidazol-1-yl, with the proviso that if G is not present and Z is a radical of the formula Ia, R₁, R₂ and R₃ cannot all be hydrogen and with the further proviso that if one of R₁, R₂ and R₃ is halo, the other two cannot both be hydrogen;
R₄ is lower alkyl, especially methyl, ethyl or isopropyl, hydroxy, aminocarbonyl, lower alkyl-carbonyl, especially methylcarbonyl, cyclohexyl, halo, especially chloro or fluoro, halo-lower alkyl, especially trifluoromethyl, lower alkoxy, especially methoxy, amino, lower alkyl-amino, especially methylamino, ethylamino, isopropylamino or tert-butylamino, di-lower alkyl-amino, especially dimethylamino, lower alkenyl-amino, especially prop-2-enylamino or but-3-enylamino, lower alkyl-carbonyl-amino, especially methylcarbonylamino, cyano, azido, hydroxy-phenyl-amino, especially 3- or 4-hydroxy-phenyl-amino, mono or tri-lower alkoxy-phenyl-amino, especially methoxy-phenyl-amino or trimethoxy-phenyl-amino, lower alkoxy-halo-phenyl-amino, especially methoxy-fluoro-phenyl-amino, phenyl-lower alkylamino, especially benzylamino, (mono or di-lower alkoxy)-phenyl-lower alkylamino, especially methoxy-benzylamino or dimethoxy-benzylamino, aminosulfonyl-phenyl-lower alkylamino, especially aminosulfonyl-benzylamino, amino-lower alkoxy-phenyl-amino, especially aminoethoxy-phenyl-amino, lower alkyl-amino-sulfonyl-lower alkyl-phenylamino, especially methylamino-sulfonylmethyl-phenylamino, lower alkyl-piperazinyl-lower alkylamino, especially 4-methylpiperazin-1-yl-propylamino, morpholinyl-lower alkylamino, especially morpholin-4-yl-propylamino, lower alkyl-piperidyl-amino, especially 1-methyl-piperidin-4-ylamino, tetrazolyl, especially 1H-tetrazol-5-yl, lower alkyl-tetrazolyl, especially lower alkyl-tetrazol-5-yl such as 1-methyl-1H-tetrazol-5-yl or 2-methyl-2H-tetrazol-5-yl, or (di-lower alkyl)-amino-lower alkyl-tetrazolyl, especially (di-lower alkyl)-amino-lower alkyl-tetrazol-5-yl such as 2-(3-dimethylaminopropyl)-2H-tetrazol-5-yl, with the proviso that if X is NH, R₄ is not aminocarbonyl or lower alkyl-carbonyl and with the further proviso that if n is l, m is 0, p is 0, r is 1, X is NH, Y₁ is O, G is not present and Z is a radical of the formula Ia, R₄, together with the benzene ring containing A and A′, does not form methylpyridinyl 2-hydroxy-pyridin-4-yl or 1-H-2-oxo-1,2-dihydropyridin-4-yl;
R₅ is most preferably hydrogen, or lower alkyl, especially methyl, or halo, especially chloro; or a tautomer thereof;
or pharmaceutically acceptable salts thereof.

In a further very preferred embodiment the invention relates the use of diaryl urea derivatives for the manufacture of pharmaceutical compositions for use in the treatment of RET dependent diseases, wherein the diaryl urea derivative is a compound of the formula I, wherein

A and A′ are both N, n is 1, m is 0, p is 0 or 2, r is 1, X is NH if p is 0, or if p is 2, X is nitrogen which together with (CH₂)₂ and the bonds represented in dotted (interrupted) lines (including the atoms to which they are bound) forms a ring, Y₁ is O, G is not present, Z is a radical of the formula Ia, at least one of R₁, R₂ and R₃ is a basic organic moiety, R₄ is amino or lower alkylamino and R₅ is hydrogen, or a tautomer thereof, or pharmaceutically acceptable salts thereof.

In another especially preferred embodiment the invention relates the use of diaryl urea derivatives for the manufacture of pharmaceutical compositions for use in the treatment of RET dependent diseases, wherein the diaryl urea derivative is a compound of the formula I*, wherein

A, A′, n, m, p, Y₁, Y₂, R₁, R₂, R₃ and R₄ have the meanings given under formula I* above, and r is 1 to 5, X is NR if p is 0, wherein R is hydrogen or an organic moiety, or if p is 2 or 3,
X is nitrogen which together with (CH₂)ₚ and the bonds represented in dotted (interrupted) lines (including the atoms to which they are bound) forms a ring, or
X is CH₂ and p is zero,
and, if p is zero, the bonds represented in dotted lines are absent;
with the proviso that if X is NH, each of R₄, independently of the others, if present, is a moiety as defined under formula I* above but not bound to the rest of formula I* via a —C(═O)—, —C(NR)— or —S(O₂)— bridge, and the substituents R₁, R₂ and R₃ are selected from the following moieties, whereby positions (o=ortho, m=meta, p=para) are indicated with regard to the position where the ring is bound to the rest of the molecule in formula I* (via the NH—C(═O)—X-moiety):
if only R₁ is other than hydrogen:
R₁=p-lower alkyl, especially p-methyl, p-ethyl, p-n-propyl:
   m-halo-lower alkyl, especially m-trifluoromethyl; or
   phenyl, p-piperidin-1-yl p-piperazin-1-yl;
      if both R₁ and R₂ are other than hydrogen:
R₁=m-halo-lower alkyl, especially m-trifluoromethyl, and R₂=p-halo, especially p-bromo;
R₁=m-halo-lower alkyl, especially m-trifluoromethyl, and R₂=p-halo-lower alkoxy, especially p-(2,2,2-trifluoroethoxy);
R₁=m-halo-lower alkyl, especially m-trifluoromethyl, and R₂=m-lower alkoxy, especially m-methoxy;
R₁=m-halo-lower alkyl, especially m-trifluoromethyl, and R₂=p-phenyl;
R₁=m-halo-lower alkyl, especially m-trifluoromethyl, and R₂=p-piperidin-1-yl or p-piperazin-1-yl;
R₁=m-halo-lower alkyl, especially m-trifluoromethyl, and R₂=p-N-morpholino or p-N-thiomorpholino;

R₁=m-lower alkoxy, especially m-methoxy, and R₂=p-halo, especially p-bromo (less preferred);
R₁=m-lower alkoxy, especially m-methoxy, and R₂=p-halo-lower alkoxy, especially p-2,2,2-trifluoroethoxy;
R₁=m-lower alkoxy, especially m-methoxy, and R₂=p-phenyl; or
R₁=m-lower alkoxy, especially m-methoxy, and R₂=p-piperidin-1-yl or p-piperazin-1-yl;
   or, if R₁, R₂ and R₃ are other than hydrogen:
R₁=m-lower alkoxy, especially m-methoxy; R₂=m-lower alkoxy, especially m-methoxy; and R₃=p-lower alkoxy, especially p-methoxy; or
R₁=lower alkoxy, especially methoxy, and R₂ and R₃ together form a lower-alkylene-dioxy, especially O—CH₂—CH₂—O—, bridge;
   and R₅ is hydrogen, lower alkyl or halo, especially hydrogen; with the proviso that if n is l, m is 0, p is 0, r is 1, X is NH and Y₁ is O, R₄, together with the benzene ring containing A and A′, does not form methylpyridinyl, 2-hydroxy-pyridin-4-yl or 1-H-2-oxo-1,2-dihydropyridin-4-yl; or a tautomer thereof; or pharmaceutically acceptable salts thereof.

In a further especially preferred embodiment the invention relates the use of diaryl urea derivatives for the manufacture of pharmaceutical compositions for use in the treatment of RET dependent diseases, wherein the diaryl urea derivative is a compound of the formula I*, wherein

A is CH, N or N→O and A′ is N or N→O, with the proviso that not more than one of A and A′ can be N→O;
n is 1;
m is 0;
p is 0, 2 or 3;
r is 1 or 2;
X is NR if p is 0, wherein R is hydrogen or lower alkyl, or if p is 2 or 3, X is nitrogen which together with (CH₂)ₚ and the bonds represented in doffed (interrupted) lines (including the atoms to which they are bound) forms a ring, or
X is CH₂ and p is zero, with the proviso that the bonds represented in dotted lines, if p is zero, are absent;

### Y₁ is O or CH₂;

R₁, R₂ and R₃ are selected from the following moieties, whereby positions (o=ortho, m=meta, p=para) are indicated with regard to the position where the ring is bound to the rest of the molecule in formula I* (via the NH—C(═O)—X-moiety): if only R₁ is other than hydrogen:
R₁=p-lower alkyl, especially p-methyl, p-ethyl, p-n-propyl;
   m-halo-lower alkyl, especially m-trifluoromethyl; or
   phenyl, p-piperidin-1-yl or p-piperazin-1-yl;
      if both R₁ and R₂ are other than hydrogen:
R₁=m-halo-lower alkyl, especially m-trifluoromethyl, and R₂=p-halo, especially p-bromo;
R₁=m-halo-lower alkyl, especially m-trifluoromethyl, and R₂=p-halo-lower alkoxy, especially p-(2,2,2-trifluoroethoxy);
R₁=m-halo-lower alkyl, especially m-trifluoromethyl, and R₂=m-lower alkoxy, especially m-methoxy;
R₁=m-halo-lower alkyl, especially m-trifluoromethyl, and R₂=p-phenyl;
R₁=m-halo-lower alkyl, especially m-trifluoromethyl, and R₂=p-piperidin-1-yl or p-piperazin-1-yl;
R₁=m-halo-lower alkyl, especially m-trifluoromethyl, and R₂=p-N-morpholino or p-N-thiomorpholino;
R₁=m-lower alkoxy, especially m-methoxy, and R₂=p-halo, especially p-bromo (less preferred):
R₁=m-lower alkoxy, especially m-methoxy, and R₂=p-halo-lower alkoxy, especially p-2,2,2-trifluoroethoxy;
R₁=m-lower alkoxy, especially m-methoxy, and R₂=p-phenyl; or
R₁=m-lower alkoxy, especially m-methoxy, and R₂=p-piperidin-1-yl or p-piperazin-1-yl;
   or, if R₁, R₂ and R₃ are other than hydrogen:
R₁=m-lower alkoxy, especially m-methoxy; R₂=m-lower alkoxy, especially m-methoxy; xy; and R₃=p-lower alkoxy, especially p-methoxy; or
R₁=lower alkoxy, especially methoxy, and R₂ and R₃ together form a lower-alkylene-dioxy, especially —O—CH₂—CH₂—O—, bridge;
   and, if r is not zero, R₄ is lower alkoxy, especially methoxy, lower alkanoylamino, especially acetylamino, hydroxyphenylamino, especially p-hydroxyphenylamino, amino-lower alkyl-oxyphenyl-amino, especially 4-[(2-aminoethyl)-oxyphenyl]-amino, sulfamoylphenylamino, especially 4-sulfamoylphenylamino, carbamoylphenylamino, especially 4-carbamoylphenylamino, [N-(hydroxy-lower alkyl)-carbamoyl]-phenylamino, especially [N-(2-hydroxyethyl)-carbamoyl]-phenylamino, or halo, especially chloro;
   and R₅ is halo, especially chloro, lower alkyl, especially methyl, or preferably hydrogen;
   or a tautomer thereof;
   or pharmaceutically acceptable salts thereof.

In another very preferred embodiment the invention relates to the use of diaryl urea derivatives for the manufacture of pharmaceutical compositions for use in the treatment of RET dependent diseases, wherein the diaryl urea derivative is a compound of the formula I selected from the Examples of WO 03/099771, or a pharmaceutically acceptable salt thereof.

Most preferably the invention relates to the NOVEL COMPOUNDS OF THE INVENTION, or pharmaceutically acceptable salts thereof.

Preferred is further the USE of the NOVEL COMPOUNDS OF THE INVENTION, or pharmaceutically acceptable salts thereof, where the protein kinase dependent disease to be treated is a protein tyrosine kinase dependent disease and especially a proliferative (preferably benign or especially malignant tumours), especially such a disease that depends on any one or more of the following protein kinases: c-Abl, Bcr-Abl, Flt-3, RET, VEGF-R, Tek, PDGF-R, FGF-R, IGF-IR, Eph receptors such as especially EphB4 receptor, c-Kit, Met, c-Src, Raf and/or ras, especially c-Abl, Bcr-Abl, Flt-3, RET, VEGF-R and/or Tek, most especially Flt-3.

### Pharmaceutical Compositions:

The invention relates also especially to pharmaceutical compositions comprising a NOVEL COMPOUND OF THE INVENTION, to the use of a NOVEL COMPOUND OF THE INVENTION in the therapeutic (in a broader aspect of the invention also prophylactic) treatment or a method of treatment of a (especially tyrosine) protein kinase dependent disease, especially the preferred diseases mentioned above, to the NOVEL COMPOUNDS OF THE INVENTION for said use and to the preparation of pharmaceutical compositions, especially for said uses.

The present invention also relates to pro-drugs of a NOVEL COMPOUND OF THE INVENTION that convert in vivo to the NOVEL COMPOUND OF THE INVENTION as such. Any reference to a NOVEL COMPOUND OF THE INVENTION is therefore to be understood as referring also to the corresponding pro-drugs of the NOVEL COMPOUND OF THE INVENTION, as appropriate and expedient.

The compounds of the present invention may be used, for example, for the preparation of pharmaceutical compositions that comprise a pharmaceutically effective amount of a compound of formula I, or a pharmaceutically acceptable salt thereof, as active ingredient, together or in admixture with a significant amount of one or more inorganic or organic, solid or liquid, pharmaceutically acceptable carriers.

The invention relates also to a pharmaceutical composition that is suitable for administration to a warm-blooded animal, especially a human (or to cells or cell lines derived from a warm-blooded animal, especially a human, e.g. lymphocytes), for the treatment or, in a broader aspect of the invention, prevention of (=prophylaxis against) a disease that responds to inhibition of protein kinase activity, especially of protein tyrosine kinase activity, especially one of the diseases mentioned above as being preferred for USE of a NOVEL COMPOUND OF THE INVENTION, comprising an amount of a NOVEL COMPOUND OF THE INVENTION, or a pharmaceutically acceptable salt thereof, which is effective for said inhibition, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to a method of treatment for a disease that responds to inhibition of an (especially tyrosine) protein kinase, especially one of the diseases mentioned above as being preferred for USE of a NOVEL COMPOUND OF THE INVENTION; which comprises administering a (against the mentioned disease) prophylactically or especially therapeutically effective amount of a NOVEL COMPOUND OF THE INVENTION, especially to a Warm blooded animal, for example a human, that, on account of one of the mentioned diseases, requires such treatment.

The dose of a compound of formula I, or a pharmaceutically acceptable salt thereof, to be administered to warm-blooded animals, for example humans of approximately 70 kg body weight, is preferably from approximately 3 mg to approximately 30 g, more preferably from approximately 10 mg to approximately 1.5 g, most preferably from about 100 mg to about 1000 mg per person per day, divided preferably into 1 to 3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilising, mixing, granulating or confectioning processes.

Solutions of the active ingredient, and also suspensions, and especially isotonic aqueous solutions or suspensions, are one preferred form used, it being possible, for example in the case of lyophilised compositions that comprise the active ingredient alone or together with a carrier, for example mannitol, for such solutions or suspensions to be produced prior to use. The pharmaceutical compositions may be sterilised and/or may comprise excipients, for example preservatives, stabilisers, wetting and/or emulsifying agents, solubilisers, salts for regulating the osmotic pressure and/or buffers, and are prepared in a manner known per se, for example by means of conventional dissolving or lyophilising processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8 to 22, especially from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E,β-carotene or 3,5-di-tert-butyl-4-hydroxytoluene. The alcohol component of those fatty acid esters has a maximum of 6 carbon atoms and is a mono- or poly-hydroxy, for example a mono, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C₈ to C₁₂, Hüls A G, Germany), but especially vegetable oils, such as cottonseed oil, almond oil, olive oil, castor oil, sesame oil, soybean oil and more especially groundnut oil.

Injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticiser, such as glycerol or sorbitol. The dry-filled capsules may comprise the active ingredient in the form of granules, for example with fillers, such as lactose, binders, such as starches, and/or glidants, such as talc or magnesium stearate, and if desired with stabilisers. In soft capsules the active ingredient is preferably dissolved or suspended in suitable oily excipients, such as fatty oils, paraffin oil or liquid polyethylene glycols, it being possible also for stabilisers and/or antibacterial agents to be added. Dyes or pigments may be added to the tablets or dragée coatings or the capsule casings, for example for identification purposes or to indicate different doses of active ingredient.

A compound of formula I, especially a NOVEL COMPOUND OF THE INVENTION, may also be used to advantage in combination with other antiproliferative agents. Such antiproliferative agents include, but are not limited to aromatase inhibitors, antiestrogens, topoisomerase I inhibitors, topoisomerase II inhibitors, microtubule active agents, alkylating agents, histone deacetylase inhibitors, farnesyl transferase inhibitors, COX-2 inhibitors, MMP inhibitors, mTOR inhibitors, antineoplastic antimetabolites, platin compounds, compounds decreasing the protein kinase activity and further anti-angiogenic compounds, gonadorelin agonists, anti-androgens, bengamides, bisphosphonates, steroids, antiproliferative antibodies, 17-(allylamino)-17-demethoxygeldanamycin (17-AAG) and temozolomide (TEMODAL®).

The term “aromatase inhibitors” as used herein relates to compounds which inhibit the estrogen production, i.e., the conversion of the substrates adrostenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™. Aminoglutethimide can be administered, e.g., in the form as it is marketed, e.g. under the trademark ORIMETEN™.

A combination of the invention comprising an antineoplastic agent which is an aromatase inhibitor is particularly useful for the treatment of hormone receptor positive breast tumours.

The term “antiestrogens” as used herein relates to compounds which antagonize the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™. Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™.

The term “topoisomerase I inhibitors” as used herein includes, but is not limited to topotecan, irinotecan, 9-nitrocamptothecin and the macromolecular camptothecin conjugate PNU-166148 (compound A1 in WO 99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™. Doxorubicin can be administered, e.g., in the form as it is marketed, e.g. under the trademark ADRIBLASTIN™. Epirubicin can be administered, e.g., in the form as it is marketed, e.g. under the trademark FARMORUBICIN™. Idarubicin can be administered, e.g., in the form as it is marketed, e.g. under the trademark ZAVEDOS™. Mitoxantrone can be administered, e.g., in the form as it is marketed, e.g. under the trademark NOVANTRON™.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D. Docetaxel can be administered, e.g., in the form as it is marketed, e.g. under the trademark TAXOTERE™. Vinblastine sulfate can be administered, e.g., in the for n as it is marketed, e.g. under the trademark VINBLASTIN R.P™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agents” as used herein includes, but is not limited to cyclophosphamide, ifosfamide and melphalan. Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™.

The term “histone deacetylase inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)-ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “farnesyl transferase inhibitors” relates to compounds which inhibit the farnesyl transferase and which possess antiproliferative activity.

The term “COX-2 inhibitors” relates to compounds which inhibit the cyclooxygenase type 2 enzyme (COX-2) and which possess antiproliferative activity such as celecoxib (Celebrex®), rofecoxib (Vioxx®) and lumiracoxib (COX189).

The term “MMP inhibitors” relates to compounds which inhibit the matrix metalloproteinase (MMP) and which possess antiproliferative activity.

The term “mTOR inhibitors” relates to compounds which inhibit the mammalian target of rapamycin (mTOR) and which possess antiproliferative activity such as sirolimus (Rapamune®), everolimus (Certican™), CCl-779 and ABT578.

The term “antineoplastic antimetabolites” includes, but is not limited to 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY64618 (LOMOTREXOL™) and OGT719.

The term “platin compounds” as used herein includes, but is not limited to carboplatin, cisplatin and oxaliplatin. Carboplatin can be administered, e.g., in the form as it is marketed, e.g. under the trademark CARBOPLAT™. Oxaliplatin can be administered, e.g., in the form as it is marketed, e.g. under the trademark ELOXATIN™.

The term “compounds decreasing the protein kinase activity and further anti-angiogenic compounds” as used herein includes, but is not limited to compounds which decrease the activity of e.g. the Vascular Endothelial Growth Factor (VEGF), the Epidermal Growth Factor (EGF), c-Src, protein kinase C, the Platelet-derived Growth Factor (PDGF), Bcr-Abl, c-Kit, Flt-3, the Insulin-like Growth Factor I Receptor (IGF-IR) and the Cyclin-dependent kinases (CDKs), and anti-angiogenic compounds having another mechanism of action than decreasing the protein kinase activity.

Compounds which decrease the activity of VEGF are especially compounds which inhibit the VEGF receptor, especially the tyrosine kinase activity of the VEGF receptor, and compounds binding to VEGF, and are in particular those compounds, proteins and monoclonal antibodies generically and specifically disclosed in WO 98/35958 (describing compounds of formula I), WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, vol. 27, no. 1, pp 14-21, 1999; in WO 00/37502 and WO 94/10202; Angiostatin™, described by M. S. O'Reilly et al, Cell 79, 1994, 315-328; and Endostatin™, described by M. S. O'Reilly et al, Cell 88, 1997, 277-285; compounds which decrease the activity of EGF are especially compounds which inhibit the EGF receptor, especially the tyrosine kinase activity of the EGF receptor, and compounds binding to EGF, and are in particular those compounds generically and specifically disclosed in WO 97/02266 (describing compounds of formula IV), EP 0 564 409, WO 99/03854, EP 0520722, EP 0 566 226, EP 0 787 722, EP 0 837 063, WO 98/10767, WO 97/30034, WO 97/49688, WO 97/38983 and, especially, WO 96/33980;

compounds which decrease the activity of c-Src include, but are not limited to, compounds inhibiting the c-Src protein tyrosine kinase activity as defined below and to SH2 interaction inhibitors such as those disclosed in WO 97/07131 and WO 97/08193;
compounds inhibiting the c-Src protein tyrosine kinase activity include, but are not limited to, compounds belonging to the structure classes of pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO 97/32879 and WO 97/49706;
compounds which decreases the activity of the protein kinase C are especially those staurosporine derivatives disclosed in EP 0 296 110 (pharmaceutical preparation described in WO 00/48571) which compounds are protein kinase C inhibitors;
compounds which decrease the activity of IGF-IR are especially those compounds disclosed in WO 02/92599;
further specific compounds that decrease protein kinase activity and which may also be used in combination with the compounds of the present invention are Imatinib (Gleevec®/Glivec®), PKC412, Iressa™ (ZD1839), {6-[4-(4-ethyl-piperazin-1-ylmethyl)-phenyl]-7H-pyrrolo[2,3-d]pyrimidin-4-yl}-((R)-1-phenyl-ethyl)-amine (AEE788) and pharmaceutically acceptable salts thereof (see also WO 03/13541), 1-(4-chloro-anilino)-4-(4-pyridyl-methyl)-phthalazine (PTK787) and pharmaceutically acceptable salts thereof (see also WO 98/35958), ZD6474, GW2016, CHIR-200131, CEP-7055/CEP-5214, CP-547632, KRN-633 and SU5416;
anti-angiogenic compounds having another mechanism of action than decreasing the protein kinase activity include, but are not limited to e.g. thalidomide (THALOMID), celecoxib (Celebrex) and ZD6126.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™. Abarelix can be formulated, e.g. as disclosed in U.S. Pat. No. 5,843,901.

The term “anti-androgens” as used herein includes, but is not limited to bicalutamide (CASODEX™), which can be formulated, e.g. as disclosed in U.S. Pat. No. 4,636,505.

The term “bengamides” relates to bengamides and derivatives thereof having antiproliferative properties.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. ‘Clodronic acid’ can be administered, e.g., in the form as it is marketed, e.g. under the trademark BONEFOS™. “Tiludronic acid” can be administered, e.g., in the form as it is marketed, e.g. under the trademark SKELID™. “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETA™.

The term “steroids” includes hydrocortisone, dexamethasone (Decadron®), methylprednisolone and prednisolone.

The term “antiproliferative antibodies” as used herein includes, but is not limited to trastuzumab (Herceptin™), Trastuzumab-DM1, erlotinib (Tarceva™), bevacizumab (Avastin™), rituximab (Rituxan®), PRO64553 (anti-CD40) and 2C4 Antibody.

For the treatment of acute myeloid leukemia (AML), the compounds of formula I, especially the NOVEL COMPOUNDS OF THE INVENTION, can be used in combination with standard leukemia therapies, especially in combination with therapies used for the treatment of AML. In particular, the compounds of the present invention can be administered in combination with e.g. farnesyltransferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The structure of the active agents identified by code nos., generic or trade names may be taken from the actual edition of the standard compendium “The Merck index” or from databases, e.g. Patents International (e.g. IMS World Publications).

The above-mentioned compounds, which can be used in combination with a compound of the present invention, can be prepared and administered as described in the art such as in the documents cited above.

### EXAMPLES

### Novel Compounds of the Invention

The following Examples serve to illustrate the invention without limiting the scope thereof.

Temperatures are measured in degrees Celsius. Unless otherwise indicated, the reactions take place at room temperature.

The R_{f} values which indicate the ratio of the distance moved by each substance to the distance moved by the eluent front are determined on silica gel thin-layer plates (Merck, Darmstadt, Germany) by thin-layer chromatography using the respective named solvent systems.

### Abbreviations:

Anal. elemental analysis (for indicated atoms, difference between calculated and measured value ≦0.4%)
aq aqueous
brine saturated solution of NaCl in water
Boc tert-butoxy carbonyl
Bu butyl
conc. Concentrated
d day(s)
DIPE diisopropyl-ether
DIPEA diisopropylethylamine
DMAP dimethylaminopyridine
DME 1,2-dimethoxyethane
DMF dimethyl formamide
DMSO-d₆ per-deuterated dimethylsulfoxide
equiv equivalent(s)
ether diethylether
EtOAc ethyl acetate
EtOH ethanol
Ex. Example
h hour(s)
HPLC high pressure liquid chromatography
l litre(s)
Me methyl
MeOH methanol
min minute(s)
m.p. melting point
MPLC medium pressure liquid chromatography
   Combi Flash system: normal phase SiO₂
   Gilson system: reversed phase Nucleosil C18 (H₂O/CH₃CN+TFA), generally product obtained as free base after neutralization with NaHCO₃
MS mass spectrum
NEt₃ triethylamine
NMR nuclear magnetic resonance
R_{f} ratio of fronts (TLC)
rt room temperature
TBDMS tert-butyl-dimethyl-silyl
tBu tert-butyl
THF tetrahydrofuran (distilled from Na/benzophenone)
TFA trifluoroacetic acid
TLC thin layer chromatography
t^{Ret} retention time (HPLC)
triphosgene bis(trichloromethyl) carbonate

### HPLC Conditions:

^{A}t_{Ret}: retension time [min] for System A: Linear gradient 20-100% CH₃CN (0.1% TFA) and H₂O (0.1% TFA) in 13 min+5 min 100% CH₃CN (0.1% TFA); detection at 215 nm, flow rate 1 ml/min at 25 or 30° C. Column: Nucleosil 120-3 C18 (125×3.0 mm).
^{B}t_{Ret}: retension time [min] for System B: Linear gradient 20-100% CH₃CN (0.1% TFA) and H₂O (0.1% TFA) in 7 min; detection at 215 nm, flow rate 1 ml/min at 25 or 30° C. Column: Nucleosil 100-3 C18 HD (125×4.0 mm).
^{C}t_{Ret}: retension time [min] for System C: Linear gradient 20-100% CH₃CN (0.1% TFA) and H₂O (0.1% TFA) in 7 min+2 min 100% CH₃CN (0.1% TFA); detection at 215 nm, flow rate 1 m/min at 30° C. Column: Nucleosil 100-3 C18HD (125×4 mm).

^{D}t_{Ret}: retension time [min] for System D: Linear gradient 20-100% CH₃CN (0.1% TFA) and H₂O (0.1% TFA) in 5 min+1.5 min 100% CH₃CN (0.1% TFA); detection at 215 nm, flow rate 1 ml/min at 30° C. Column: Nucleosil 100-3 C18HD (70×4 mm).

### Example 1

### N-[4-(6-Chloro-pyrimidin-4-yloxy)-phenyl]-N′-[3-(azetidin-1-ylmethyl)-5-trifluoromethyl-phenyl]-urea

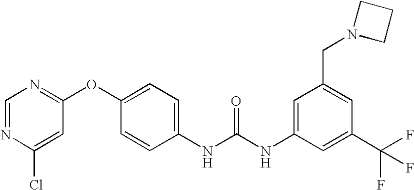

To a solution of 935 mg (3.78 mMol) of 4-chloro-6-(4-isocyanato-phenoxy)-pyrimidine (Step 1.3) in 3 ml of THF under N₂Atmosphere, 870 mg (3.78 mMol) of 3-(azetidin-1-ylmethyl)-5-trifluoromethyl-aniline (Step 1.6) dissolved in 20 ml of ether are added. After stirring for 3 h at rt, the reaction mixture is partially concentrated in vacuo, diluted with ether, whereby the title compound crystallized and can be filtered off and washed with ether: MS: [M+1]⁺=478; ¹H-NMR (CDCl₃): 8.58 (s, 1H), 7.61 (s, 1H), 7.46 (s, 1H), 7.44 (d, 8.6 Hz, 2H), 7.24 (s, 1H), 7.12 (d, 8.6 Hz, 2H), 6.93 (s, 1H), 6.89 (s, 1H), 6.81 (s, 1H), 3.59 (s, 2H), 3.24 (t, 7.0 Hz, 2×2H), 2.11 (q, 7.0 Hz, 2H).

The starting material is prepared as follows:

### Step 1.1: 4-Chloro-6-(4-nitro-phenoxy)-pyrimidine

To an ice-cooled solution of 214 g (5.35 Mol) NaOH dissolved in 6.5 l of H₂O, 744 g (5.35 Mol) of 4-nitrophenol are added. Then a solution of 797 g (5.35 Mol) of 4,6-dichloro-pyrimidine in 6.5 l of acetone is added dropwise during 60 min and the mixture is stirred for 18 h at 65° C. The reaction mixture is cooled to 10° C., the precipitated crude product filtered off and washed with 400 ml H₂O acetone 1:1: m.p.: 127-128° C.; Anal. C₁₀H₆ClN₃O₃: C, H, N, Cl, O; MS: [M]⁺=251; ¹H-NMR (DMSO-d₆): 8.70 (s, 1H, pyrimidinyl), 8.34 (d, 9 Hz, 2H, phenyl), 7.59 (s, 1H, pyrimidinyl), 7.57 (d, 9 Hz, 2H, phenyl).

### Step 1.2: 4-(6-Chloro-pyrimidin-4-yl-oxy)-aniline

1095 g (4.3 Mol) of 4-chloro-6-(4-nitro-phenoxy)-pyrimidine dissolved in 10 l of MeOH/THF 2:1 is hydrogenated in the presence of 33 g Raney-Ni at rt for 4 h. The reaction solution is filtered and concentrated. Crystallization from EtOAc gives the title compound: Anal. C₁₀H₈ClN₃O: C, H, N, Cl, O; MS: [M+1]⁺=222; ¹H-NMR (DMSO-d₆): 8.60 (s, 1H), 7.12 (s, 1H), 6.86 (d, 9 Hz, 2H, phenyl), 6.57 (d, 9 Hz, 2H, phenyl), 5.13 (s, 2H, NH₂).

### Step 1.3: 4-Chloro-6-(4-isocyanato-phenoxy)-pyrimidine

Apparatus: 18 litre reaction vessel, dropping funnel and condenser. A phosgene solution (20% in toluene, 1.43 l; 2.9 Mol) diluted with 10 l of toluene under N₂-atmosphere is cooled to approximately −20° C. Then a solution of 250 g (1.12 Mol) of 4-(6-chloro-pyrimidin-4-yl-oxy)-aniline in 4.4 l of CH₂Cl₂ is added during 30 min. The resulting suspension is heated to distil off approximately 4.5 l of solvent. Distillation is continued (boiling point: 110° C.) giving a clear solution (≈3 l) in the reaction vessel, which is cooled to rt and concentrated in vaccuo. Distillation of the resulting waxy crude product at 0.2 mbar gives the title compound as a solid: m.p.: 103° C.

### Step 1.4: (3-Nitro-5-trifluoromethyl-phenyl)-(azetidin-1-yl)-methanone

In an ice bath under N₂-atmosphere, 9.77 g (41.6 mMol) of 3-nitro-5-trifluoromethyl-benzoic acid (Lancaster), 150 ml CH₂Cl₂, a few drops of DMF and 5.8 ml (67 mMol) of oxalylchloride are mixed and then stirred for 17 h at rt. The resulting solution is concentrated in vacuo. The residue is dissolved in 50 ml CH₂Cl₂ and added dropwise to an ice cooled solution of 5.9 ml (87 mMol) azetidine in 50 ml CH₂Cl₂. After stirring for 15 min, the mixture is washed with 1 N HCl, a diluted solution of Na₂CO₃, water and brine. The aqueous layers are re-extracted twice with EtOAc, the combined organic phases dried (Na₂SO₄) and concentrated. Crystallization from hexane gives the title compound; m.p.: 91° C.; MS: [M+1]⁺=275.

### Step 1.5: (3-Amino-5-trifluoromethyl-phenyl)-(azetidin-1-yl)-methanone

Hydrogenation of 10.39 g (37.9 mMol) of (3-nitro-5-trifluoromethyl-phenyl)-(azetidin-1-yl)-methanone in 200 ml ethanol in the presence of 2 g of Raney-Nickel, filtration through celite, partial concentration of the filtrate and dilution with hexane gives the crystalline title compound; m.p.: 154° C.; MS: [M+1]⁺=245.

### Step 1.6: 3-(Azetidin-1-ylmethyl)-5-trifluoromethyl-aniline

To 8.62 g (35.3 mMol) (3-amino-5-trifluoromethyl-phenyl)-(azetidin-1-yl)-methanone in 75 ml THF under N₂-atmosphere cooled in an ice-bath, 10.6 ml (95%; 106 mMol) of BH₃.Me₂S in 15 ml THF are added dropwise. The resulting solution is stirred for 2 d at rt and then 4 h at 65° C. After cooling to rt, 50 ml of HCl conc./H₂O 1:1 is added and the mixture stirred for 15 h at rt and 7 h at 65° C. The mixture is poured off into EtOAc and a 10% solution of Na₂CO₃, the aqueous phase separated off and extracted twice with EtOAc. The organic layers are washed twice with water and brine, dried (Na₂SO₄) and concentrated. Column chromatography (SiO₂; EtOAc/EtOH 95:5→EtOAc/EtOH/Et₃N 95:5:1) yields the title compound; m.p. 60-61° C.; MS: [M+1]⁺=231.

### Example 2

### N-[4-(6-Methylamino-pyrimidin-4-yloxy)-phenyl]-N′-(3-azetidin-1-ylmethyl-5-trifluoromethyl-phenyl)-urea

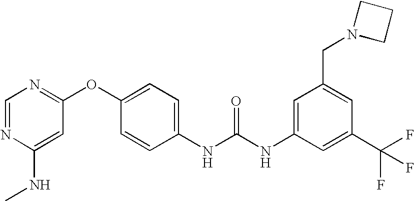

Under N₂-Atmosphere, 250 mg (0.52 mMol) of N-[4-(6-chloro-pyrimidin-4-yloxy)-phenyl]-N′-(3-azetidin-1-ylmethyl-5-trifluoromethyl-phenyl)-urea in 3 ml of a 33% solution of MeNH₂ in EtOH are stirred in an ice-bath for 4 h. Then ≈1 g of SiO₂ is added to the solution and the mixture concentrated in vacuo. The resulting powder is put on top of a MPLC column (SiO₂) and eluted with MeOH (+1% NH₃^{aq})/CH₂Cl₂ 3:97-1:9→1:4, yielding the title compound: MS: [M+1]⁺=473; ¹H-NMR (CD₃OD+CDCl₃): 8.11 (s, 1H), 7.95 (m, 1H), 7.46 (s, 1H), 7.45 (d, 7.4 Hz, 2H), 7.17 (s, 1H), 7.03 (d, 7.4 Hz, 2H), 5.59 (s, 1H), 3.90 (s, 2H), 3.63 (m, 2×2H), 2.81 (s, H₃C), 2.30 (m, 2H).

### Example 3

### N-[4-(6-Azido-pyrimidin-4-yloxy)-phenyl]-N′-(3-azetidin-1-ylmethyl-5-trifluoromethyl-phenyl)-urea

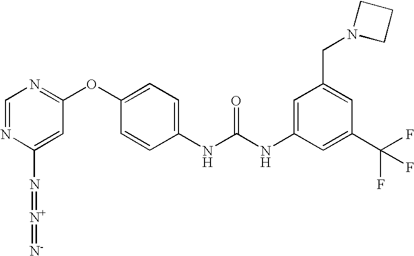

A mixture of 300 mg (0.63 mMol) of N-[4-(6-chloro-pyrimidin-4-yloxy)-phenyl]-N′-(3-azetidin-1-ylmethyl-5-trifluoromethyl-phenyl)-urea and 82 mg (1.26 mMol) NaN₃ in 5 ml of DMF is stirred for 16 h at 40° C. and 5 h at 60° C. The reaction mixture is poured into water and extracted with 3 portions of EtOAc. The organic layers are washed with water and brine, dried (Na₂SO₄) and concentrated. The residue is re-dissolved in 20 ml of THF, filtered and the filtrate directly used in the hydrogenation step of Ex. 4. The title compound can be obtained by concentration of the filtrate in vacuo: MS: [M+1]⁺=485; ¹H-NMR (CDCl₃): 8.53 (s, 1H), 7.96 (s, 1H), 7.94 (s, 1H), 7.57 (s, 1H), 7.53 (s, 1H), 7.45 (d, 8.6 Hz, 2H), 7.17 (s, 1H), 7.04 (d, 8.6 Hz, 2H), 6.25 (s, 1H), 3.58 (s, 2H), 3.27 (t, 7.0 Hz, 2×2H), 2.11 (q, 7.0 Hz, 2H).

### Example 4

### N-[4-(6-Amino-pyrimidin-4-yloxy)-phenyl]-N′-(3-azetidin-1-ylmethyl-5-trifluoromethyl-phenyl)-urea

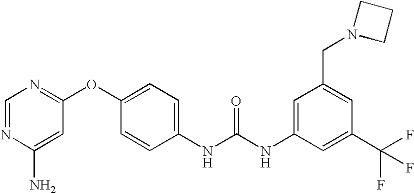

A solution of N-[4-(6-azido-pyrimidin-4-yloxy)-phenyl]-N′-(3-azetidin-1-ylmethyl-5-trifluromethyl-phenyl)-urea (0.63 mMol) in 20 ml of THF is hydrogenated in the presence of 60 mg Pd/C 10%. After filtering off of the catalyst, ≈1 g of SiO₂ is added to the filtrate and the mixture concentrated in vacuo. The resulting powder is put on top of a MPLC column (SiO₂) and eluted with EtOH (+1% NEt₃)/EtOAc 1:49→4:46→1:4, yielding the title compound: MS: [M+1]⁺=459; ¹H-NMR (CD₃OD): 8.08 (s, 1H), 7.82 (s, 1H), 7.54 (s, 1H), 7.52 (d, 9.0 Hz, 2H), 7.24 (s, 1H), 7.09 (d, 9.0 Hz, 2H), 5.75 (s, 1H), 3.68 (s, 2H), 3.35 (t, 7.2 Hz, 2×2H), 2.16 (q, 7.2 Hz, 2H).

### Example 5

### N-[4-(6-Chloro-pyrimidin-4-yloxy)-phenyl]-N′-[3-(4-isopropylpiperazin-1-ylmethyl)-5-trifluoromethyl-phenyl]-urea

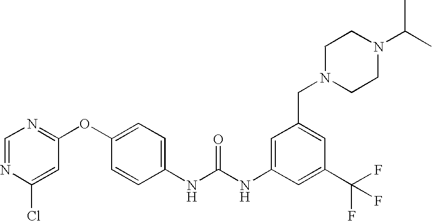

A solution of 1.00 g (4.0 mMol) of 4-chloro-6-(4-isocyanato-phenoxy)-pyrimidine (Step 1.3) dissolved in 3 ml THF is added dropwise to a solution of 1.31 g (4.3 mMol) of 3-(4-isopropylpiperazin-1-ylmethyl)-5-trifluoromethyl-aniline (Step 5.3) in 33 ml ether under N₂-atmosphere. After stirring for 4 h at rt, the reaction mixture is concentrated in vacuo. Column chromatography (SiO₂; CH₂Cl₂/MeOH 9:1→88:12→85:15) gives the title compound: m.p.: 101° C.; MS: [M+1]⁺=549; ¹H-NMR (CDCl₃): 8.57 (s, 1H), 7.64 (s, 1H), 7.48 (s, 1H), 7.47 (d, 9 Hz, 2H), 7.28 (s, 1H), 7.19 (m, 1H), 7.13 (s, 1H), 7.12 (d, 9 Hz, 2H), 6.92 (s, 1H), 3.49 (s, 2H), 2.69 (sept, 6.3 Hz, 1H), 2.58 (m, 4H), 2.52 (m, 4H), 1.08 (d, 6.3 Hz, 6H).

The starting material is prepared as follows:

### Step 5.1: (3-Nitro-5-trifluoromethyl-phenyl)-(4-isopropylpiperazin-1-yl)-methanone

In an ice bath under N₂-atmosphere, 9.00 g (38.3-mMol) of 3-nitro-5-trifluoromethyl-benzoic acid (Lancaster), 150 ml CH₂Cl₂, a few drops of DMF and 5.3 ml (61 mMol) of oxalylchloride are mixed and then stirred for 4.5 h at rt. The resulting solution is concentrated in vacuo. The residue is dissolved in 50 ml CH₂Cl₂ and added dropwise to an ice cooled solution of 10.3 g (80 mMol) of 1-isopropylpiperazine in 50 ml CH₂Cl₂. After stirring for 140 min, the mixture is washed with a diluted solution of Na₂CO₃, water and brine. The aqueous layers are re-extracted twice with EtOAc, the combined organic phases dried (Na₂SO₄) and concentrated. Crystallization from DIPE/hexane gives the title compound: m.p.: 70.71° C.; MS: [M+1]⁺346.

### Step 5.2: (3-Amino-5-trifluoromethyl-phenyl)-(4-isopropylpiperazin-1-yl)-methanone

Hydrogenation of 9.2 g (27 mMol) of (3-nitro-5-trifluoromethyl-phenyl)-(4-isopropylpiperazin-1-yl)-methanone in 200 ml ethanol in the presence of 2 g of Raney-Nickel as described in Step 1.5 gives the title compound: m.p.: 89-90° C.; MS: [M+1]⁺=316.

### Step 5.3: 3-(4-Isopropylpiperazin-1-ylmethyl)-5-trifluoromethyl-aniline

To 7.0 g (22 mMol) of (3-amino-5-trifluoromethyl-phenyl)-(4-isopropylpiperazin-1-yl)-methanone in 70 ml THF under N₂-atmosphere, 67 ml (1M in THF; 67 mMol) of BH₃.THF are added dropwise. The resulting solution is stirred for 18 h at rt, then 100 ml of HCl conc./H₂O 1:1 are added and the mixture is stirred for 5 h at rt. The reaction mixture is extracted with EtOAc, the organic phase washed with 0.1 N HCl and discarded. To the acidic aqueous layers then 250 ml of saturated Na₂CO₃ solution are added, followed by extraction with 3 portions of EtOAc. The organic layers are washed with brine, dried (Na₂SO₄) and concentrated, yielding the title compound as an oil: MS: [M+1]⁺=302; ¹H-NMR(CDCl₃): 6.93 (s, 1H), 6.82 (s, 1H), 6.77 (s, 1H), 3.82 (s, H₂N), 3.45 (s, 2H), 2.67 (sept, 6.3 Hz, 1H), 2.57 (m, 4H), 2.51 (m, 4H), 1.07 (d, 6.3 Hz, 6H).

### Example 6

### N-[4-(6-Methylamino-pyrimidin-4-yloxy)-phenyl]-N′-[3-(4-isopropylpiperazin-1-ylmethyl)-5-trifluoromethyl-phenyl]-urea

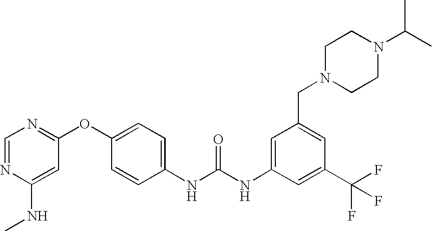

Under N₂-Atmosphere, 368 mg (0.67 mMol) of N-[4-(6-chloro-pyrimidin-4-yloxy)-phenyl]-N′-[3-(4-isopropylpiperazin-1-ylmethyl)-5-trifluoromethyl-phenyl]-urea in 3 ml of a 33% solution of MeNH₂ in EtOH are stirred in an ice-bath for 4.5 h. The mixture is poured off into EtOAc and an aqueous solution of NaHCO₃, the aqueous phase separated off and extracted twice with EtOAc. The organic layers are washed twice with water and brine, dried (Na₂SO₄) and concentrated. Reversed phase chromatography gives the title compound: MS: [M+1]⁺=544; ¹H-NMR (CD₃OD): 8.15 (s, 1H), 7.84 (s, 1H), 7.66 (s, 1H), 7.56 (d, 9 Hz, 2H), 7.34 (s, 1H), 7.13 (d, 3 Hz, 2H), 5.72 (s, 1H), 3.63 (s, 2H), 2.87 (s, H₃C), 2.9-2.5 (m, 9H) 1.15 (d, 6.7 Hz, 6H).

### Example 7

### N-[4-(6-Azido-pyrimidin-4-yloxy)-phenyl]-N′-[3-(4-isopropylpiperazin-1-ylmethyl)-5-trifluoromethyl-phenyl]-urea

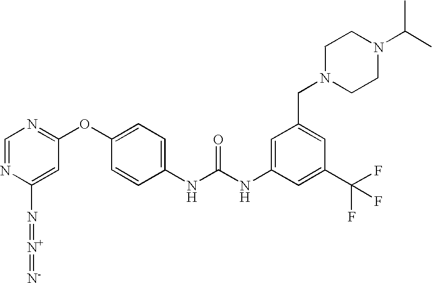

A mixture of 470 mg (0.86 mMol) of N-[4-(6-chloro-pyrimidin-4-yloxy)-phenyl]-N′-[3-(4-isopropylpiperazin-1-ylmethyl)-5-trifluoromethyl-phenyl]-urea and 111 mg (1.7 mMol) NaN₃ in 7 ml of DMF is stirred for 2 h at 80° C. Then the solution is cooled in an ice-bath and poured into 80 ml of water under vigorous stirring. Filtration of the resulting suspension and washing with water gives the title compound: MS: [M+1]⁺=556; HPLC ^{A}t_{Ret}=11.2.

### Example 8

### N-[4-(6-Amino-pyrimidin-4-yloxy)-phenyl]-N′-[3-(4-isopropylpiperazin-1-ylmethyl)-5-trifluoromethyl-phenyl]-urea

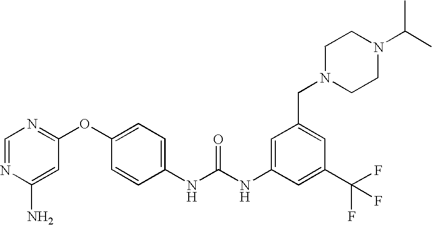

A solution of 0.39 g (0.70 mMol) N-[4-(6-azido-pyrimidin-4-yloxy)-phenyl]-N′-[3-(4-isopropylpiperazin-1-ylmethyl)-5-trifluoromethyl-phenyl]-urea in 20 ml of THF is hydrogenated in the presence of 100 mg Pd/C₅%. The catalyst is filtered off, the filtrate concentrated in vacuo, the residue re-dissolved in CH₂Cl₂/MeOH and after adding ≈1 g of SiO₂ again concentrated. The resulting powder is out on top of a MPLC column (SiO₂) and eluted with EtOAc/EtOH (+1% NEt₃) 19:1→9:1→7:3, yielding the title compound after crystallization from hexane: Anal. C₂₆H₃₀N₇F₃O₂ 0.8 H₂O 0.2 EtOAc: C, H, N, H₂O; MS: [M+1]⁺=530; ¹H-NMR (CD₃OD): 8.12 (s, 1H), 7.86 (s, 1H), 7.63 (s, 1H), 7.57 (d, 8.6 Hz, 2H), 7.34 (s, 1H), 7.13 (d, 8.6 Hz, 2H), 5.79 (s, 1H), 3.62 (s, 2H), 2.8-2.5 (m, 9H), 1.13 (d, 6.7 Hz, 6H).

### Example 9

### N-[4-(6-Chloro-Pyrimidin-4-yloxy)-phenyl]-N′-[3-(4-methylpiperazin-1-ylmethyl)-5-trifluoromethyl-phenyl]-urea

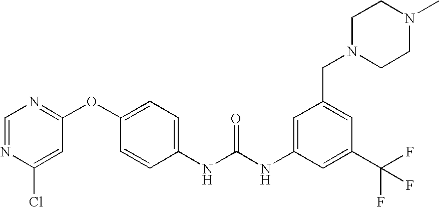

1.00 g (4.0 mMol) of 4-chloro-6-(4-isocyanato-phenoxy)-pyrimidine (Step 1.3) dissolved in 3 ml THF and 1.1 g (4.0 mMol) of 3-(4-methylpiperazin-1-ylmethyl)-5-trifluoromethyl-aniline (Step 9.3) in 30 ml ether are converted analogously to Ex. 5 into the title compound: m.p.: 291-292° C.; Anal. C₂₄H₂₄N₆ClF₃O₂ 0.5H₂O: C, H, N, Cl, F; MS: [M+1]⁺=521.

The starting material is prepared as follows:

### Step 9.1: (3-Nitro-5-trifluoromethyl-phenyl)-(4-methylpiperazin-1-yl)-methanone

Analogously to Step 5.1, 9.00 g (38.3 mMol) of 3-nitro-5-trifluoromethyl-benzoic acid are activated with 5.3 ml (61 mMol) of oxalylchloride and reacted with 8.9 ml (80 mMol) of 1-methylpiperazine, yielding the title compound as an oil; MS: [M+1]⁺=318; HPLC ^{A}t_{Ret}=8.7.

### Step 9.2: (3-Amino-5-trifluoromethyl-phenyl)-(4-methylpiperazin-1-yl)-methanone

Hydrogenation of 11.8 g (37 mMol) of (3-nitro-5-trifluoromethyl-phenyl)-(4-methylpiperazin-1-yl)-methanone in 200 ml ethanol in the presence of 2 g of Raney-Nickel as described in Step 1.5 gives the title compound; m.p.: 114-115° C.; MS: [M+1]⁺=288.

### Step 9.3: 3-(4-Methylpiperazin-1-ylmethyl)-5-trifluoromethyl-aniline

Analogously to Step 1.6, 9.91 g (34.5 mMol) (3-amino-5-trifluoromethyl-phenyl)-(4-methylpiperazin-1-yl)-methanone in 90 ml THF are reduced by BH₃-Me₂S to the title compound: m.p.: 98-99° C.; MS: [M+1]⁺=274; ¹H-NMR (CDCl₃): 6.94 (s, 1H), 6.82 (s, 1H), 6.78 (s, 1H), 3.82 (s, H₂N), 3.45 (s, 2H), 2.48 (m, 8H), 2.30 (s, H₃C).

The compounds of Ex. 10-13 can be prepared analogously to the procedures described herein:

### Example 10

### N-[4-(6-Chloro-pyrimidin-4-yloxy)-phenyl]-N′-(3-diethylaminomethyl-5-trifluoromethyl-phenyl)-urea

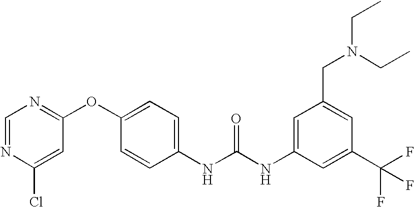

171 mg (0.69 mMol) of 4-chloro-6-(4-isocyanato-phenoxy)-pyrimidine (Step 1.3) dissolved in 2 ml THF and 170 mg (0.69 mMol) of 3-diethylamino-methyl)-5-trifluoromethyl-aniline (Step 10.3) in 6 ml ether are converted analogously to Ex. 5 into the title compound. MS: [M+1]⁺=493.9.

The starting material is prepared as follows:

### Step 10.1: (3-Nitro-5-trifluoromethyl-phenyl)-(diethylamino)-methanone

Analogously to Step 5.1, 2.40 g (10.0 mMol) of 3-nitro-5-trifluoromethyl-benzoic acid are activated with 1.7 ml (20.0 mMol) of oxalylchloride and reacted with 7.3 g (100 mMol) of diethylamine, yielding the title compound as an oil; MS: [M−1]⁺=290; ¹H-NMR (DMSO-d₈): 8.79 (s, 1H), 8.41 (s, 1H), 8.21 (s, 1H), 3.50 (q, 2H), 3.21 (q, 2H), 1.19 (t, 3H), 1.01 (t, 3H).

### Step 10.2: (3-Amino-5-trifluoromethyl-phenyl)-(4-methylpiperazin-1-yl)-methanone

Hydrogenation of 2.8 g (9.6 mMol) of (3-nitro-5-trifluoromethyl-phenyl)-(diethylamino)-methanone in 50 ml ethanol in the presence of 140 mg of Pd—C described in Step 1.5 gives the title compound as a yellow solid; MS: [M+1]⁺=261. ¹H-NMR (DMSO-d₆): 6.89 (s, 1H), 6.78 (s, 1H), 6.60 (s, 1H), 5.79 (s, 2H, NH₂), 3.50-3.39 (m, 2H), 3.25-3.02 (m, 2H), 1.21-0.99 (m, 6H).

### Step 10.3: 3-(Diethylamino methyl)-5-trifluoromethyl-aniline

Analogously to Step 1.6, 1.04 g (4.0 mMol) (3-amino-5-trifluoromethyl-phenyl)-(4-methylpiperazin-1-yl)-methanone in 15 ml THF are reduced by BH₃.Me₂s to the title compound: MS: [M+1]⁺=247; ¹H-NMR (DMSO-d₆): 6.87 (s, 1H), 6.84 (s, 1H), 6.81 (s, 1H), 5.60 (s, 2H, NH₂), 2.75-2.65 (m, 4H), 1.28-1.08 (m, 6H).

### Example 11

### N-[4-(6-Methylamino-pyrimidin-4-yloxy)-phenyl]-N′-(3-diethylaminomethyl-5-trifluoromethyl-phenyl)-urea

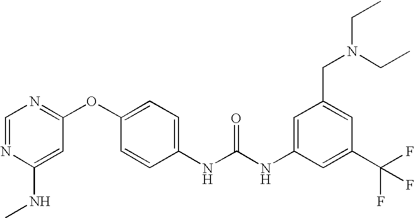

Under N₂-Atmosphere, 250 mg (0.52 mMol) of N-[4-(6-chloro-pyrimidin-4-yloxy)-phenyl]-N′-(3-diethylaminomethyl-5-trifluoromethyl-phenyl)-urea in 3 ml of a 33% solution of MeNH₂ in EtOH are stirred at 5° C. for 2 h. After aqueous workup the crude product is purified by flash chromatography (SiO₂, gradient CH₂Cl₂/MeOH 0-40%) yielding the title compound: m.p.: 68-70° C.; MS: [M+1]⁺=489; ¹H-NMR (DMSO-d₆): 9.21 (s, 1H, NH), 8.83 (s, 1H, NH), 8.09 (s, 1H), 7.85 (s, 1H), 7.45 (d, 2H), 7.20 (s, 1H), 7.05 (d, 2H), 5.71 (s, 1H), 3.56 (s, 2H), 2.74 (s, 3), 2.50-2.32 (m, 4H), 1.01-0.95 (m, 6H).

### Example 12

### N-[4-(6-Azido-pyrimidin-4-yloxy)-phenyl]-N′-(3-diethylaminomethyl-5-trifluoromethyl-phenyl)-urea

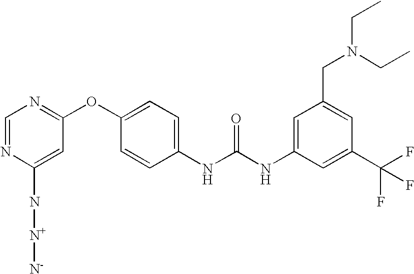

A mixture of 218 mg (0.44 mMol) of N-[4-(6-chloro-pyrimidin-4-yloxy)-phenyl]-N′-(3-diethylaminomethyl-5-trifluoromethyl-phenyl)-urea and 50 mg (0.7 mMol) NaN₃ in 6 ml of DMF is stirred for 2 h at 80° C. Then the reaction mixture is diluted with ethyl acetate and washed with brine. The organic layer is separated, dried and concentrated to give the crude product which is purified by flash chromatography (SiO₂, gradient CH₂Cl₂/MeOH 040%). MS: [M+1]⁺=501.

### Example 13

### N-[4-(6-Amino-pyrimidin-4-yloxy)-phenyl]-N′-(3-diethylaminomethyl-5-trifluoromethyl-phenyl)-urea

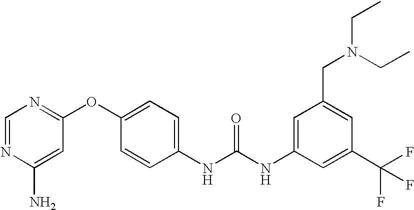

A solution of 98 mg (0.17 mMol) N-[4-(6-azido-pyrimidin-4-yloxy)-phenyl]-N′-(3-diethylaminomethyl-5-trifluoromethyl-phenyl)-urea in 10 ml of DME is hydrogenated in the presence of 20 mg Pd/C₅%. The catalyst is filtered off, the filtrate concentrated in vacuo, the residue is purified by preparative TLC (SiO₂, CH₂C)₂/MeOH 9:1) yielding the title compound: m.p.: 63-65° C. MS: [M+1]⁺=475.

### Example 14

### N-[4-(6-Chloro-pyrimidin-4-yloxy)-phenyl]-N′-[4-(4-methylpiperazin-1-ylmethyl)-3-trifluoromethyl-phenyl]-urea

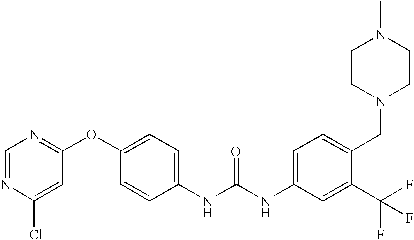

To an ice-cooled solution of 687 mg (2.77 mMol) 4-chloro-6-(4-isocyanato-phenoxy)-pyrimidine (Step 1.3) dissolved in 3 ml THF under N₂-atmosphere, a solution of 758 mg (2.77 mMol) of 4-(4-methylpiperazin-1-ylmethyl)-3-trifluoromethyl-aniline (Step 14.4) in 20 ml ether is added dropwise. After stirring for 3 h at rt, the resulting suspension is filtered and the residue washed with ether, yielding the title compound: MS: [M+1]⁺=521; ¹H-NMR (CDCl₃): 8.55 (s, 1H), 7.67 (d, 8.6 Hz, 1H), 7.56 (d, 8.6 Hz, 1H), 7.54 (s, 1H), 7.41 (d, 9 Hz, 2H), 7.21 (s, 1H), 7.15 (s, 1H), 7.08 (d, 9 Hz, 2H), 6.91 (s, 1H), 3.58 (s, 2H), 2.48 (m, 8H), 2.30 (s, H₃C).

The starting material is prepared as follows:

### Step 14.1: N-(4-Methyl-3-trifluoromethyl-phenyl)-2,2,2-trifluoro-acetamide

To an ice-cooled solution of 320 g (1.827 Mol) of 5-amino-2-methylbenzotrifluoride and 1.47 l (18.27 Mol) pyridine in 4.5 l of CH₂Cl₂ under N₂-atmosphere, 284 ml (2.01 Mol) of trifluoroacetic acid anhydride are added dropwise. After 50 min, the mixture is diluted with 5 l ice-cooled 2 N HCl. The organic phases are separated off and washed two times with 2 l cold 2 N HCl, then 112 N HCl and finally with 2 l brine. The aqueous layers are extracted twice with CH₂Cl₂, the organic phases dried (Na₂SO₄) and concentrated partially. Crystallization by addition of hexane yields the title compound: m.p.: 72-73° C.

### Step 14.2: N-(4-Bromomethyl-3-trifluoromethyl-phenyl)-2,22-trifluoro-acetamide

To a solution of 60.9 g (224.6 mMol) of N-(4-methyl-3-trifluoromethyl-phenyl)-2,2,2-trifluoro-acetamide in 830 ml ⁿbutyl acetate under N₂-atmosphere, 44 g (247 mMol) N-bromosuccinimide and 830 mg (5 mMol) azo-iso-butyronitrile are added. The suspension is heated up to 60° C. and then illuminated for 30 min by a Phillips low-voltage lamp (500 W; 10500 lm), whereby the temperature rises to 70-75° C. and a clear brown solution is formed. There is still remaining educt detectable, therefore another 22 g N-bromosuccinimide are added in 3 portions. After totally 6 h illumination, the resulting solid is filtered off and discarded and the filtrate concentrated. The residue is distributed between 2 l CH₂Cl₂ and 1 l H₂O and the aqueous layer extracted with 1 l CH₂Cl₂. The organic phases are washed 4 times with 1 l H₂O, 0.5 l brine, dried (Na₂SO₄) and concentrated. Column chromatography (SiO₂; hexane/CH₂Cl₂ 2:1→1:1) and crystallization from CH₂Cl₂/hexane yields the title compound: m.p.: 119-120° C.

### Step 14.3: 2,2,2-Trifluoro-N-[4-(4-methyl-piperazin-1-ylmethyl)-3-trifluoromethyl-phenyl]-acetamide

To an ice-cooled solution of 1.9 ml (17.1 mMol) N-methylpiperazine in 50 ml acetonitrile under N₂-atmosphere, a solution of 2.00 g (5.71 mMol) N-(4-bromomethyl-3-trifluoromethylphenyl)-2,2,2-trifluoro-acetamide in 50 ml acetonitrile is added dropwise during 30 min. After additional 20 min, the reaction mixture is concentrated in vacuo. The resulting oil is diluted with EtOAc and saturated NaHCO₃-solution/H₂O 1:1. The aqueous layer is separated off and extracted twice with EtOAc. The organic layers are washed with saturated NaHCO₃-solution/H₂O 1:1, water and brine, dried (Na₂SO₄), concentrated and directly used in Step 14.4: MS: [M+1]⁺=370; HPLC ^{A}t_{Ret}=9.5.

### Step 14.4: 4-(4-Methylpiperazin-1-ylmethyl)-3-trifluoromethyl-aniline

To a solution of 1.102 g (2.98 mMol) of 2,2,2-trifluoro-N-[4-(4-methyl-piperazin-1-ylmethyl)-3-trifluoromethyl-phenyl]-acetamide in 26 ml of boiling methanol, 14 ml of a 1 M solution of K₂CO₃ in water are added dropwise. After 1 h stirring, the reaction mixture is cooled to rt and diluted with EtOAc and water. The aqueous layer is separated off and extracted twice with EtOAc. The organic phases are washed with water and brine, dried (Na₂SO₄) and concentrated to yield the title compound, which is directly used in Ex. 14: MS: [M+1]⁺=274; HPLC ^{A}t_{Ret}=5.4.

Alternative synthesis for 4-(4-methylpiperazin-1-ylmethyl)-3-trifluoromethyl-aniline:

Step 14.4.1: 4-Nitro-2-trifluoromethyl-benzoic acid [see: J. Gen. Chem. USSR (Engl. Transl.) 33 (1963), 2957]

Under N₂-atmosphere, a mechanically stirred mixture of 50 g (263 mMol) o-trifluoromethyl-benzoic acid and 307 ml H₂SO₄ 96% is cooled in an ice bath. Then 105 ml HNO₃ 100% is added dropwise at 5-7° C. during 75 min. The ice bath is removed and stirring continued for 2 h at rt. The reaction mixture is poured into 1.9 kg ice and stirred for 20 min. Filtration of the suspension, washing with 100 ml cold water and drying (0.2 mbar, 50° C.) gives the crude title compound containing 20% of a regio-isomer. This material is partially dissolved in 0.4 l boiling toluene and filtered. The filtrate is concentrated to half of its volume, then 0.1 l hexane is added. Upon cooling to rt, the title compound crystallizes and can be filtered off: m.p.: 138-141° C.; ¹H-NMR (CDCl₃): 8.71 (d, 2.3 Hz, 1H), 8.56 (dd, 2.3 Hz, 8.2 Hz, 1H), 8.18 (d, 8.2 Hz, 1H).

### Step 14.4.2: (4-Nitro-2-trifluoromethyl-phenyl)-(4-methylpiperazin-1-yl)-methanone

To an ice-cooled solution of 17.99 g (76.5 mMol) of 4-nitro-2-trifluoromethyl-benzoic acid, 300 ml CH₂Cl₂ and 3 ml DMF under N₂-atmosphere, 12.3 ml (145 mMol) of oxalylchloride are added dropwise. After 4.5 h, the resulting solution is concentrated in vacuo. The residue is dissolved in 300 ml CH₂Cl₂ and added dropwise to an ice cooled solution of 17.8 ml (160 mMol) of 1-methylpiperazine in 120 ml CH₂Cl₂. After stirring for 3 h, the mixture is diluted with 0.5 l CH₂Cl₂, washed with 3 portions of a 10% solution of Na₂CO₃, water and brine. The organic phase is dried (Na₂SO₄) and concentrated to the title compound as an oil: MS: [M+1]⁺=318; ¹H-NMR (CDCl₃): 8.62 (d, 2.3 Hz, 1H), 8.50 (dd, 2.3 Hz, 8.2 Hz, 1H), 7.60 (d, 8.2 Hz, 1H), 3.90 (m, 1H), 3.84 (m, 1H), 3.21 (t, 5.1 Hz, 2H), 2.53 (t, 5.1 Hz, 2H), 2.36 (s, 3H), 2.36 (m, 2H).

### Step 14.4.3: (4-Amino-2-trifluoromethyl-phenyl)(4-methylpiperazin-1-yl)-methanone

A solution of 24 g (76 mMol) (4-nitro-2-trifluoromethyl-phenyl)-(4-methylpiperazin-1-yl)-methanone in 400 ml ethanol is hydrogenated for 14 h in the presence of 4 g of Raney-Nickel. The catalyst is filtered off and the filtrate concentrated in vacuo. The residue in 500 ml boiling toluene is filtered, the filtrate concentrated partially until the product starts to crystallize. Cooling to rt and filtration affords the title compound: m.p.: 154-156° C.; MS: [M+1]⁺=288.

### Step 14.4.4: 4-(4-Methylpiperazin-1-ylmethyl)-3-trifluoromethyl-aniline

To 17.2 g (60 mMol) of (4-amino-2-trifluoromethyl-phenyl)-(4-methylpiperazin-1-yl)-methanone in 160 ml THF under N₂-atmosphere, 180 ml (1 M in THF; 180 mMol) of BH₃-THF are added during 75 min. The resulting solution is stirred for 18 h at rt, then 180 ml of HCl conc./H₂O 1:1 are added under cooling and the mixture is stirred for 18 h at rt. The reaction mixture is concentrated partially, the residue extracted with EtOAc, the separated organic phase washed with 0.1 N HCl and discarded. Then 0.7 l of a saturated Na₂CO₃ solution are added to the acidic aqueous layers (e pH 9-10), followed by extraction with 3 portions of EtOAc. The organic phases are washed with brine, dried (Na₂SO₄) and concentrated. Crystallization from boiling toluene gives the title compound: m.p.: 119-121° C.

### Example 15

### N-[4-(6-Chloro-pyrimidin-4-yloxy)-phenyl]-N′-[4-(4-isopropylpiperazin-1-ylmethyl)-3-trifluoromethyl-phenyl]-urea

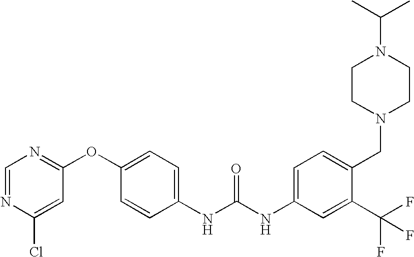

To an ice-cooled solution of 1.251 g (5.05 mMol) 4-chloro-6-(4-isocyanato-phenoxy)-pyrimidine (Step 1.3) dissolved in 4 ml THF under N₂-atmosphere, a solution of 1.522 g (5.05 mMol) of 4-(4-isopropylpiperazin-1-ylmethyl)-3-trifluoromethyl-aniline (Step 15.2) in 25 ml ether is added dropwise. After stirring for 2.5 h, the reaction mixture is diluted with ether, the solid filtered off and washed with ether. The crude product is re-dissolved in CH₂Cl₂/MeOH, absorbed on SiO₂, which then is put on top of a SiO₂ chromatography column. Eluation with CH₂Cl₂/MeOH/NH₃^{aq} 95:5:1 yields the title compound: Anal. C₂₆H₂₈N₆ClF₃O₂ 0.5H₂O: C, H, N, F; MS: [M+1]⁺=549; ¹H-NMR (CDCl₃): 8.56 (s, 1H), 7.68 (d, 8 Hz, 1H), 7.57 (d, 8 Hz, 1H), 7.56 (s, 1H), 7.43 (d, 9 Hz, 2H), 7.11 (s, 1H), 7.10 (d, 9 Hz, 2H), 7.05 (s, 1H), 6.92 (s, 1H), 3.58 (s, 2H), 2.67 (sept, 6.3 Hz, 1H), 2.56 (m, 4H), 2.51 (m, 4H), 1.08 (d, 6.3 Hz, 6H).

The starting material is prepared as follows:

### Step 15.1: 2,2,2-Trifluoro-N-[4-(4-isopropyl-piperazin-1-ylmethyl)-3-trifluoromethyl-phenyl]-acetamide

To an ice-cooled solution of 3.46 g (27 mMol) N-isopropylpiperazine in 70 ml acetonitrile under N₂-atmosphere, a solution of 3.15 g (9.0 mMol) N-(4-bromomethyl-3-trifluoromethylphenyl)-2,2,2-trifluoro-acetamide (Step 14.2) in 70 ml acetonitrile is added dropwise during 35 min. After additional 5 min, a workup procedure as described in Step 14.3 gives the title compound as an oil: MS: [M+1]⁺=398; HPLC ^{A}t_{Ret}=10.1.

### Step 15.2: 4-(4-Isopronylpiperazin-1-ylmethyl)-3-trifluoromethyl-aniline

To a solution of 3.58 g (9.0 mMol) of 2,2,2-trifluoro-N-[4-(4-isopropyl-piperazin-1-ylmethyl)-3-trifluoromethyl-phenyl]-acetamide in 90 ml of boiling methanol, 45 ml of a 1 M solution of K₂CO₃ in water are added dropwise. After 110 min stirring, the reaction mixture is cooled to rt and concentrated partially in vacuo. The residue is diluted with EtOAc and water, the aqueous layer separated off and extracted twice with EtOAc. The organic phases are washed with water and brine, dried (Na₂SO₄) and concentrated partially. Upon dilution with hexane, the title compound crystallizes and can be isolated by filtration: m.p.: 117-119° C.; MS: [M+1]⁺=302.

### Example 16

### N-[4-(6-Methylamino-pyrimidin-4-yloxy)-phenyl]-N′-[4-(4-isopropylpiperazin-1-ylmethyl)-3-trifluoromethyl-phenyl]-urea trifluoroacetate

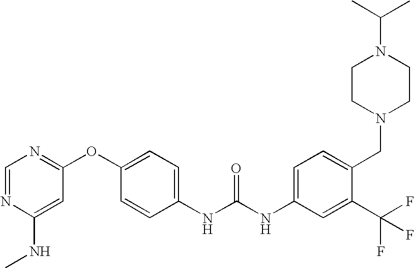

Under N₂-Atmosphere, 450 mg (0.82 mMol) of N-[4-(6-chloro-pyrimidin-4-yloxy)-phenyl]-N′-[4-(4-isopropylpiperazin-1-ylmethyl)-3-trifluoromethyl-phenyl]-urea in 4 ml of a 33% solution of MeNH₂ in EtOH are stirred in an ice-bath for 3 h. The mixture is poured off into EtOAc and a 10% solution of NaHCO₃, the aqueous phase separated off and extracted twice with EtOAc. The organic layers are washed twice with water and brine, dried (Na₂SO₄) and concentrated. Reversed phase chromatography gives the title compound: MS: [M+1]⁺=544; ¹H-NMR (DMSO-d₆): 9.16 (s, HN), 9.04 (m, HN⁺), 8.93 (s, HN), 8.12 (m, 1H), 7.95 (s, 1H), 7.62 (2s, 2H), 7.48 (d, 9 Hz, 2H), 7.33 (m, HNMe), 7.05 (d, 9 Hz, 2H), 5.73 (s, 1H), 3.65 (s, 2H), 3.47 (m, 1H), 3.39 (m, 2H), 3.00 (m, 2H), 2.95 (m, 2H), 2.76 (m, H₃C), 2.39 (m, 2H), 1.26 (d, 7 Hz, 6H).

### Example 17

### N-[4-(6-Methylamino-pyrimidin-4-yloxy)-phenyl]-N′-[4-(4-isopropyl-4-oxy-piperazin-1-ylmethyl)-3-trifluoromethyl-phenyl]-urea trifluoroacetate

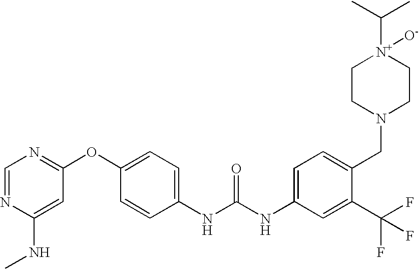

The title compound can be isolated as a slower moving side product during the reversed phase chromatography of the reaction mixture of Ex. 16: MS: [M+1]⁺=560; ¹H-NMR (DMSO-d₆): 11.48 (s, HN), 9.14 (s, HN), 8.92 (s, HN), 8.11 (m, 1H), 7.95 (s, 1H), 7.63 (m, 2H), 7.47 (d, 8 Hz, 2H), 7.30 (m, HNMe), 7.05 (d, 8 Hz, 2H), 5.73 (s, 1H), 3.95 (sept, 7 Hz, 1H), 3.69 (s, 2H), 3.60 (m, 4H), 2.87 (m, 2H), 2.76 (m, H₃C), 2.7 (m, 2H), 1.35 (d, 7 Hz, 6H).

### Example 18

### N-[4-(6-Azido-Pyrimidin-4-yloxy)-phenyl]-N′-[4-(4-isopropylpiperazin-1 ylmethyl)-3-trifluoromethyl-phenyl]-urea

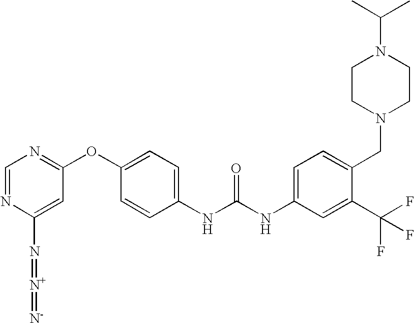

The title compound is prepared from 647 mg (1.18 mMol) of N-[4-(6-chloro-pyrimidin-4-yloxy)-phenyl]-N′-[4-(4-isopropylpiperazin-1-ylmethyl)-3-trifluoromethyl-phenyl]-urea as described in Ex. 7: MS: [M+1]⁺=556; HPLC ^{A}t_{Ret}=11.4.

### Example 19

### N-[4-(6-Amino-pyrimidin-4-yloxy)-phenyl]-N′-[4-(4-isopropylpiperazin-1-ylmethyl)-3-trifluoromethyl-phenyl]-urea

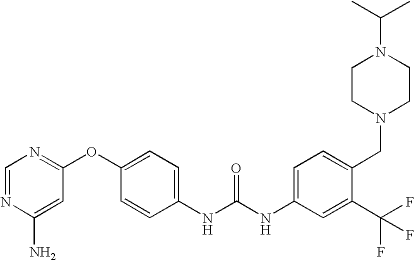

Hydrogenation of 0.66 g (1.18 mMol) of N-[4-(6-azido-pyrimidin-4-yloxy)-phenyl]-N′-[4-(4-isopropylpiperazin-1-ylmethyl)-3-trifluoromethyl-phenyl]-urea in 25 ml THF in the presence of 0.12 g Pd/C-10% (“Engelhard 4505”), filtration, concentration of the filtrate and chromatography [MPLC: CH₂Cl₂/MeOH (+1% NH₃^{aq}) 199:1→93:7→82:18] gives the title compound: Anal. C₂₆H₃₀N₇F₃O₂ 0.8H₂O: C, H, N, F; MS: [M+1]⁺=530; ¹H-NMR (CDCl₃): 8.25 (s, 1H), 7.86 (s, 1H), 7.65 (d, 8.2 Hz, 1H), 7.56 (m, 3H), 7.25 (d, 8 Hz, 2H), 6.97 (d, 8 Hz, 2H), 5.64 (s, 1H), 5.26 (s, H₂N), 3.57 (s, 2H), 2.64 (sept, 6.7 Hz, 1H), 2.53 (m, 4H), 2.49 (m, 4H), 1.06 (d, 6.7 Hz, 6H).

The compounds of Ex. 19-1 and 19-2 can be prepared analogously to the procedures described herein:

### Example 19-1

### N-[4-(6-Amino-pyrimidin-4-yloxy)-phenyl]-N′-[4-(4-H-piperazin-1-ylmethyl)-5-trifluoromethyl-phenyl]-urea

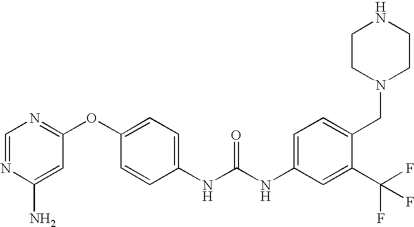

Hydrogenation of 0.33 g (0.68 mMol) of N-[4-(6-azido-pyrimidin-4-yloxy)-phenyl]-N′-[4-(4-benzoyloxycarbonylpiperazin-1-ylmethyl)-3-trifluoromethyl-phenyl]-urea in 10 ml DME in the presence of 0.05 g Pd/C 10% (“Engelhard 4505”, filtration, concentration of the filtrate and chromatography [C18: CH₃CN/H₂O (+0.1% TFA)] gives the title compound: m.p.: 153-155° C. MS: [M+1]⁺=488; ¹H-NMR (DMSO-d₆): 9.39 (s, 1H), 9.17 (s, 1H), 8.59 (s, 2H, NH), 8.18 (s, 1H), 7.98 (s, 1H), 7.59 (s, 1H), 7.42 (d, 2H), 7.01 (d, 2H); 5.62 (s, 2H), 3.17-3.08 (m, 4H), 2.62-2.52 (m, 4H).

The starting material is prepared as follows:

### Step 19-1.1: 2,2,2-Trifluoro-N-[4-(4-benzoyloxycarbonyl-piperazin-1-ylmethyl)-3-trifluoromethyl-phenyl]-acetamide

To a solution of 1.57 g (7.1 mMol) N-benzyl-1-piperazine carboxylate in 10 ml EtOH under N₂-atmosphere, a solution of 1.0 g (2.8 mMol) N-(4-bromomethyl-3-trifluoromethyl-phenyl)-2,2,2-trifluoro-acetamide (Step 14.2) in 5 ml EtOH is added dropwise during 35 min. After additional 30 min of stirring and a workup procedure as described in Step 14.3 the title compound is obtained as an oil: MS: [M+1]⁺=491; ¹H-NMR (CDCl₃): 8.15 (s, 1H, NH), 7.81-6.99 (m, 3H), 7.39-7.28 (m, 5H), 5.15 (s, 2H), 3.59 (s, 2H), 3.52-3.43 (m, 4H), 2.44-2.39 (m, 4H).

### Step 19-1.2: 4-(4-Benzoyloxycarbonyl-piperazin-1-ylmethyl)-3-trifluoromethyl-aniline

To a solution of 1.31 g (2.67 mMol) of 2,2,2-trifluoro-N-[4-(4-benzyloxycarbonyl-piperazin-1-ylmethyl)-3-trifluoromethyl-phenyl]-acetamide in 20 ml of boiling methanol, 13 ml of a 1 M solution of K₂CO₃ in water are added dropwise. After 1 h stirring, the reaction mixture is cooled to rt and diluted with EtOAc and water. The aqueous layer is separated off and extracted twice with EtOAc. The organic phases are washed with water and brine, dried (Na₂SO₄) and concentrated to yield the title compound, which is directly used in Step 19-1.3: [M+1]⁺=394; ¹H-NMR (DMSO-d₆): 7.39-7.21 (m, 6H), 6.82 (s, 1H), 6.75 (d, 1H), 5.41 (s, 2H), 5.01 (s, 2H), 3.40-3.29 (m, 6H), 2.31-2.24 (m, 4H).

### Step 19-1.3: N-[4-(6-Chloro-pyrimidin-yloxy)-phenyl]-N′-[4-(4-benzoyloxycarbonylpiperazin-1-ylmethyl)-3-trifluoromethyl-phenyl]-urea

To an ice-cooled solution of 0.38 g (1.52 mMol) 4-chloro-6-(4-isocyanato-phenoxy)-pyrimidine (Step 1.3) dissolved in 5 ml THF under N₂-atmosphere, a solution of 0.60 g (1.52 mMol) of 4-(4-benzoyloxy carbonylpiperazin-1-ylmethyl)-3-trifluoromethyl-aniline (Step 15.2) in 15 ml ether is added dropwise. After stirring for 1.5 h, the reaction mixture is diluted with ether, the solid filtered off and washed with ether. The crude product is re-dissolved in CH₂Cl₂/MeOH, absorbed on SiO₂, which then is put on top of a SiO₂ chromatography column. Eluation with CH₂Cl₂/MeOH; gradient 0-3% MeOH yields the title compound: MS: [M+1]⁺=642.7; ¹H-NMR (CDCl₃): 8.59 (s, 1H), 7.62 (d, 1H), 7.59-7.51 (m, 2H), 7.41 (d, 2H), 7.35-7.30 (m, 3H), 7.18 (s, 1H), 7.15 (d, 2H), 7.05 (s, 1H), 6.90 (s, 1H), 5.19 (s, 2H), 3.62 (s, 2H), 3.59-3.40 (m, 4H), 2.51-2.38 (m, 4H).

### Step 19-1.4: N-[4-(6-Azido-pyrimidin-4-yloxy)-phenyl]-N′-[4-(benzoyloxycarbonylpiperazin-1-ylmethyl)-3-trifluoromethyl-phenyl]-urea

The title compound is prepared from 300 mg (0.46 mMol) of N-[4-(6-chloro-pyrimidin-4-yloxy)-phenyl]-N′-[4-(4-benzyloxycarbonyllpiperazin-1-ylmethyl)-3-trifluoromethyl-phenyl]-urea as described in Ex. 7: MS: [M+1]⁺=648; ¹H-NMR (CDCl₃): 8.58 (s, 1H), 8.01 (s, 1H), 7.69-7.59 (m, 3H), 7.41 (d, 1H), 7.39-7.35 (m, 5H), 7.20 (s, 1H), 7.09 (d, 2H), 6.25 (s, 1H), 5.17 (s, 2H) 3.61 (s, 2H), 3.59-3.42 (m, 4H), 2.43-2.38 (m, 4H).

### Example 19-2

### N-[4-(6-Methylamino-pyrimidin-4-yloxy)-phenyl]-N′-[4-(4-H-piperazin-1-ylmethyl)-5-trifluoromethyl-phenyl]-urea

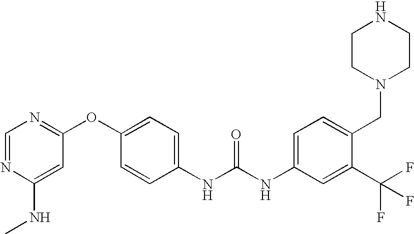

Hydrogenation of 88.0 mg (0.14 mMol) of N-[4-(6-methylamino-pyrimidin-4-yloxy)-phenyl]-N′-[4-(4-benzoyloxycarbonylpiperazin-1-ylmethyl)-3-trifluoromethyl-phenyl]-urea in 5 ml MeOH in the presence of 15 mg Pd/C 10% (“Engelhard 4505”), filtration, concentration of the filtrate and chromatography [C18: CH₃CN/H₂O (+0.1% TFA) gives the title compound: m.p.: 197-198° C.; MS: [M+1]⁺=502; ¹H-NMR (DMSO-d₆): 8.80 (s, 1H, NH), 8.52 (s, 1H, NH), 8.06 (s, 1H), 7.89 (s, 1H), 7.63 (d, 1H), 7.58 (d, 1H), 7.49 (d, 2H), 7.05 (d, 2H), 5.79 (s, 1H), 3.18-3.09 (m, 4H), 2.80 (s, 3H), 2.69-2.59 (m, 4H).

The starting material is prepared as follows:

### Step 19-2.1: N-[4-(6-Methylamino-pyrimidin-4-yloxy)-phenyl]-N′-[4-(4-benzoyloxy carbonylpiperazin-1-ylmethyl)-3-trifluromethyl-phenyl]urea

Under N₂-Atmosphere, 122 mg (0.19 mMol) of N-[4-(6-chloro-pyrimidin-4-yloxy)-phenyl]-N′-[4-(4-benzyloxycarbonyl-piperazin-1-ylmethyl)-3-trifluoromethyl-phenyl]-urea (Ex. 20-1) in 4 ml of a 33% solution of MeNH₂ in EtOH are stirred in an ice-bath for 2 h. The mixture is poured off into EtOAc and a 10% solution of NaHCO₃, the aqueous phase separated off and extracted twice with EtOAc. The organic layers are washed twice with water and brine, dried (Na₂SO₄) and concentrated. Flash chromatography (SiO₂, CH₂Cl₂/MeOH, gradient 0-5% MeOH) gives the title compound: MS: [M+1]⁺=636; ¹H-NMR (CDCl₃): 8.21 (s, 1H), 7.61-7.44 (m, 3H), 7.39-7.31 (m, 5H), 7.17-6.99 (m, 3H), 6.51 (d, 1H), 5.75 (s, 1H), 5.12 (s, 2H), 3.59 (s, 3H), 3.48-3.41 (m, 4H), 2.91 (s, 2H), 2.41-2.35 (m, 4H).

### Example 20

### N-[4-(6-Chloro-pyrimidin-4-yloxy)-phenyl]-N′-[4-(4-^{tert}butylpiperazin-1-ylmethyl)-3-trifluoromethyl-phenyl]-urea

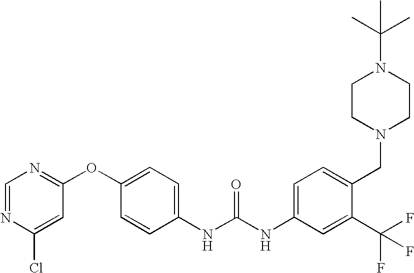

Prepared in analogy to Ex. 14. The crude product is purified by flash chromatography (SiO₂, CH₂Cl₂/MeOH, gradient 0-10% MeOH) to give the title compound as a yellow foam. C₂₇H₃₀ClF₃N₆O₂; MS (ES+), M+H=563.6; ¹H-NMR (300 MHz, CDCl₃): 8.59 (s, 1H), 7.62 (d, 1H), 7.60-7.56 (m, 2H), 7.42 (d, 2H), 7.18-7.11 (m, 3H), 7.02 (s, 1H), 3.79 (s, 2H), 2.78-2.54 (m, 4H), 2.51-2.40 (m, 4H), 1.04 (s, 9H).

The starting material is prepared as follows:

### Step 20.1: Bis-(2-chloro-ethyl)-carbamic acid ethyl ester

The title compound is prepared from bis-(2-chloroethyl)amine according to a literature procedure [J. Pharmaceutical. Sci. 61 (1972), 1316]. C₇H₁₃Cl₂NO₂; MS (ES+), M+H=216.4; ¹H-NMR (300 MHz, CDCl₃): 4.19 (q, 2H), 3.75-3.58 (m, 8H), 1.14 (t, 3H).

### Step 20.2: 4-tert-Butyl-piperazine-1-carboxylic acid ethyl ester

The compound of Step 20.1 (10 g, 46 mmol) is dissolved in tert-butanol and subsequently NaI (280 mg, 1.8 mmol) and tert-butylamine (5.12 g, 70 mmol) are added at rt. The yellow reaction mixture is then heated to 130° C. in an oil bath and stirred for 13 h. It is allowed to cool to rt again and K₂CO₃ (6.9 g. 50 mmol) is added. The reaction is then exposed to microwave irradiation (130° C./6 min). The product is collected by filtration, taken up in EtOAc and purified by acid/base washing to give the title compound as a yellow oil. (2.54 g, 32 mmol, 26%). C₁₁H₂₂N₂O₂; MS (ES+), M+H=215.5; ¹H-NMR (300 MHz, CDCl₃): 4.15 (q, 2H), 3.51-3.40 (m, 4H), 2.58-2.41 (m, 4H) 1.12 (t, 3H), 1.02 (s, 9H).

### Step 20.3: 1-tert-Butyl-piperazine

The compound of Step 20.2 (1 g, 4.6 mmol) is dissolved in ethanol (15 mL). KOH (1.2 g, 201 mmol) is added and the reaction is heated to reflux for 12 h. It is allowed to cool to rt and concentrated under reduced pressure. The residue is taken up in EtOAc and washed with brine. Organic layers are dried over Na₂SO₄, concentrated and dried under high vacuum to give the title compound as a yellow oil. (546 mg, 3.7 mmol, 82%). C₈H₁₈N₂; MS (ES+), M+H=143.5; ¹H-NMR (300 MHz, CDCl₃): 2.91-2.84 (m, 4H), 2.59-2.48 (m, 4H), 1.02 (s, 9H).

### Step 20.4: N-[4-(4-tert-Butyl-piperazin-1-ylmethyl)-3-trifluoromethyl-phenyl]-2,2,2-trifluoro-acetamide

The compound of Step 20.3 (540 mg, 3.8 mmol) is dissolved in EtOH (3 mL) and 532 mg (1.5 mmol) N-(4-bromomethyl-3-trifluoromethyl-phenyl)-2,2,2-trifluoro-acetamide (Step 14.2) is added at rt. The reaction is stirred at ambient temperature for 1.5 h until completion. It is concentrated and the residual crude product is purified by flash chromatography (SiO₂; CH₂Cl₂/MeOH, gradient 0-8% MeOH) to give the title compound as a yellow oil (654 mg, 1.5 mmol, 42%). C₁₈H₂₃F₆N₃O; MS (ES+), M+H=412.0.

### Step 20.5: 4-(4-tert-Butyl-piperazin-1-ylmethyl)-3-trifluoromethyl-phenyl-amine

The compound of Step 20.4 (650 mg, 1.5 mmol) is dissolved in MeOH (15 mL) and treated with K₂CO₃ (7.9 mL of a 1N aqueous solution) at rt. The reaction is heated to reflux for 1 h until completion, cooled back to rt and concentrated. The residual oil is taken up in EtOAc and washed with brine. The organic layers are dried over Na₂SO₄, filtered and concentrated under reduced pressure. Drying under high vacuum gives the title compound as a yellow oil (496 mg, 1.5 mmol). C₁₆H₂₄F₃N₃; MS (ES+), M+H=316.1; ¹H-NMR (300 MHz, CDCl₃): 7.44 (d, 1H), 6.91 (d, ₁H), 6.79 (d, d, 1H), 3.79 (bs, 2H), 3.51 (s, 2H), 2.67-2.59 (m, 4H), 2.58-2.40 (m, 4H), 1.01 (s, 9H).

The compounds of Ex. 20-1 to 20-8 can be prepared analogously to the procedures described herein:

### Example 20-1

### N-[4-(6-Chloro-pyrimidin-4-yloxy)-phenyl]-N′-[4-(4-benzoyloxycarbonylpiperazin-1-ylmethyl)-3-trifluoromethyl-phenyl]-urea

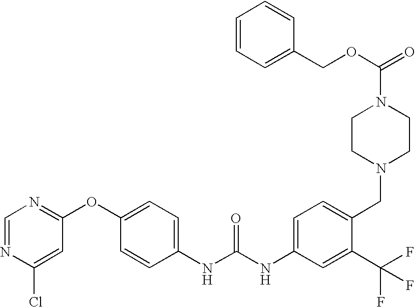

Prepared in analogy to Ex. 14 from 600 mg (1.5 Mmol) 4-(4-amino-2-trifluoromethyl-benzyl)-piperazine-1-carboxylic acid benzyl ester. The crude product is purified by flash chromatography (SiO₂, CH₂Cl₂/MeOH, gradient 0-10% MeOH). MS (ES+), M+H=643; ¹H-NMR (300 MHz, CDCl₃): 8.57 (s, 1H), 7.64 (d, 1H, J=8.2 Hz), 7.59-7.55 (m, 2H), 7.43 (d, J=8.7 Hz), 7.36-7.32 (m, 3H), 7.17 (s, 1H), 7.08 (d, J=8.7 Hz), 7.04 (s, 1H), 6.91 (s, 1H), 5.17 (s, 2H), 3.60 (s, 2H), 3.57-3.45 (m, 4H), 2.49-2.33 (m, 4H).

The starting material is prepared as follows:

### Step 20-1,1: 4-[4-(2,2,2-Trifluoro-acetylamino)-2-trifluoromethyl-benzyl]-piperazine-1-carboxylic acid benzylester

A solution of 1.0 g (2.8 mMol) N-(4-bromomethyl-3-trifluoromethyl-phenyl)-2,2,2-trifluoro-acetamide (Step 14.2) in 15 ml EtOH is treated with 1.57 g (7.1 mMol) benzyl-1-piperazinecarboxylate at rt. The reaction is stirred for 1 h at rt. After completion it is concentrated and the residual crude product purified by flash chromatography (SiO₂, CH₂Cl₂/MeOH, gradient 0-10% MeOH) to give the title compound as a yellow solid. MS (ES+), M+H=491; ¹H-NMR (300 MHz, CDCl₃): 8.19 (s, 1H, NH), 7.92-7.89 (m, 3H), 7.40-7.38 (m, 5H), 5.18 (s, 2H), 3.60 (s, 2H), 3.58-3.52 (m, 4H), 2.49-2.38 (m, 4H).

### Step 20-1.2: 4-(4-Amino-2-trifluoromethyl-benzyl)-piperazine-1-carboxylic acid benzyl ester

A solution of 1.3 g (2.6 mMol) 4-[4-(2,2,2-trifluoro-acetylamino)-2-trifluoromethyl-benzyl]-piperazine-1-carboxylic acid benzylester in 20 ml MeOH is treated with 13.4 ml 1M aqueous solution of K₂CO₃ at rt. The reaction is then heated to reflux and stirred for 2 h. After completion MeOH is destilled off and the residual aqueous suspension is extracted with EtOAc (3×). Combined organic extracts are dried over Na₂SO₄ and after filtration and concentration in vacuo the title compound is obtained as a yellow solid. MS (ES+), M+H=394; ¹H-NMR (300 MHz, DMSO-d6): 7.39-7.29 (m, 6H), 6.82 (s, 1H), 6.74 (d, 1H); 5.41 (s, 2H; NH₂), 5.02 (s, 2H), 3.42 (s, 2H), 3.40-3.31 (m, 4H), 2.31-2.24 (m, 4H).

### Example 20-2

### N-[4-(6-Chloro-pyrimidin-4-yloxy)-phenyl]-N′-[4-(N,N-dimethylamino-methyl)-3-trifluoromethyl-phenyl]-urea

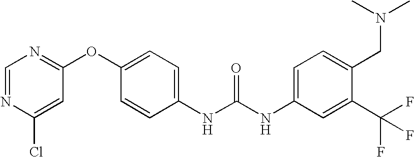

Prepared in analogy to Ex. 14 starting from 110 mg (0.5 mMol) of 4-(4-(N,N-dimethylaminomethyl)-3-trifluoromethyl-phenyl-amine and 125 mg (0.5 mMol) 4-chloro-6-(4-isocyanatophenoxy)-pyrimidine (Step 1.3). The crude product is purified by flash chromatography (SiO₂, CH₂Cl₂/MeOH, gradient 0-10% MeOH) to give the title compound as a yellow foam. m.p. 98-105° C. MS (ES+), M+H=466. ¹H-NMR (300 MHz, DMSO-d₆): 9.02 (s, 1H), 8.92 (s, 1H), 8.60 (s, 1H), 7.97 (s, 1H), 7.59-7.54 (m, 2H), 7.49 (d, 2H), 7.38 (s, 1H), 7.12 (d, 2H), 3.41 (s, 2H), 2.19 (s, 6H).

The starting material is prepared as follows:

### Step 20-2.1: 4-(4-(N,N-Dimethylamino-methyl)-3-trifluoromethyl-phenyl-2,2,2-trifluoro-acetamide

501 mg (1.5 mmol) N-(4-bromomethyl-3-trifluoromethyl-phenyl)-2,2,2-trifluoro-acetamide (Step 14.2) is added to 5 ml of a solution of dimethyl amine in EtOH (33%) at rt. The reaction is stirred at ambient temperature for 0.5 h until completion. It is concentrated and the residual crude product is purified by flash chromatography (SiO₂; CH₂Cl₂/MeOH, gradient 0-5% MeOH) to give the title compound as a yellow oil. MS (ES+), M+H=315.

### Step 20-2.2: 4-(4-(N,N-Dimethylamino-methyl)-3-trifluoromethyl-phenyl-amine

The compound of Step 20-1.1 (359 mg, 1.2 mmol) is dissolved in MeOH (12 mL) and treated with K₂CO₃ (6 mL of a 1N aqueous solution) at rt. The reaction is heated to reflux for 1.5 h until completion, cooled back to rt and concentrated. The residual oil is taken up in EtOAc and washed with brine. The organic layers are dried over Na₂SO₄, filtered and concentrated under reduced pressure. Drying under high vacuum gives the title compound as a yellow oil. M+H=219.

### Example 20-3

### N-[4-(6-Chloro-pyrimidin-4-yloxy)-phenyl]-N′-[4-(N,N-diethylamino-methyl)-3-trifluoromethyl-phenyl]-urea

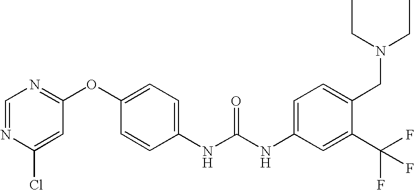

Prepared in analogy to Ex. 14. starting from 370 mg (1.5 mMol) of 4-(4-(N,N-diethylaminomethyl)-3-trifluoromethyl-phenyl-amine and 371 mg (1.5 mMol) 4-chloro-6-(4-isocyanatophenoxy)-pyrimidine (Step 1.3). The crude product is purified by flash chromatography (SiO₂, CH₂Cl₂/MeOH, gradient 0-10% MeOH) to give the title compound: MS (ES+), M+H=494.

### Example 20-4

### N-[4-(6-Chloro-pyrimidin-4-yloxy)-phenyl]-N′-[4-[(3-dimethylamino-propyl)-methyl-amino-methyl)]-3-trifluoromethyl-phenyl]-urea

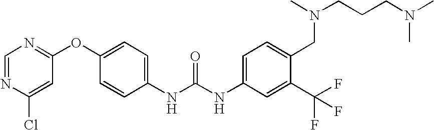

Prepared in analogy to Ex. 14 starting from 600 mg (2.2 mMol) of 4-[(3-dimethylaminopropyl)-methyl-amino-methyl)]-3-trifluoromethyl-phenyl-amine and 539 mg (2.2 mMol) 4-chloro-6-(4-isocyanato-phenoxy)-pyrimidine (Step 1.3) to give the title compound: MS (ES+), M+H=523.

### Example 20-5

### N-[4-(6-Chloro-pyrimidin-4-yloxy)-phenyl]-N′-[4-[(4-cyano-benzyl)-amino-methyl]-3-trifluoromethyl-phenyl]-urea

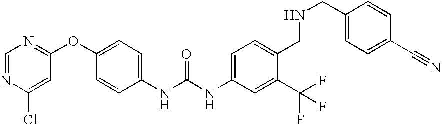

Prepared in analogy to Ex. 14 starting from 440 mg (1.4 mMol) of 4-[(4-cyano-benzyl)-amino-methyl)]-3-trifluoromethyl-phenyl-amine and 375 mg (1.4 mMol) 4-chloro-6-(4-isocyanato-phenoxy)-pyrimidine (Step 1.3) to give the title compound: MS (ES+), M+H=553.

### Example 20-6

### N-[4-(6-Chloro-pyrimidin-4-yloxy)-phenyl]-N′-[4-(1-morpholinyl)-3-trifluoromethyl-phenyl]-urea

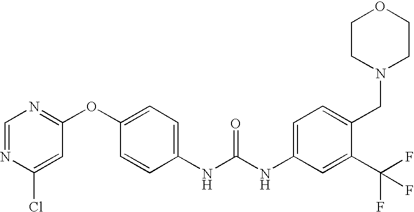

Prepared in analogy to Ex. 14 starting from 260 mg (1.0 mMol) of 4-(morpholin-4-ylmethyl)-3-trifluromethyl-phenylamine and 248 mg (1.0 mMol) 4-chloro-6-(4-isocyanato-phenoxy)-pyrimidine (Step 1.3) to give the title compound: MS (ES+), M+H=508. ¹H-NMR (300 MHz, DMSO-d₆): 8.82 (s, 1H, NH), 8.79 (s, 1H, NH), 8.69 (s, 1H), 7.91 (s, 1H), 7.75-7.65 (2×d, 2H), 7.50 (d, 2H), 7.15 (d, 2H), 7.12 (s, 1H), 3.74 (s, 2H), 3.71-3.61 (m, 4H), 2.62-2.52 (m, 4H).

### Example 20-7

### N-[4-(6-Chloro-pyrimidin-4-yloxy)-phenyl]-N′-[4-(pyrrolidin1-yl-amino-methyl)-3-trifluoromethyl-phenyl]-urea

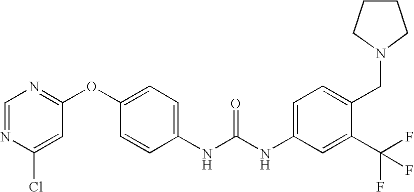

### Prepared in Analogy to Ex. 14.

MS (ES+), M+H=493. ¹H-NMR (300 MHz, CDCl₃): 8.59 (s, 1H), 7.71 (d, 2H), 7.51-7.39 (m, 3H), 7.17 (s, 1H), 7.02 (d, 2H), 6.93 (s, 1H), 3.79 (s, 2H), 2.62-2.58 (m, 4H), 2.93-2.72 (m, 4H).

### Example 20-8

### N-[4-(6-Chloro-pyrimidin-4-yloxy)-phenyl]-N′-[4-(4-(4-methoxybenzyl)-piperazin-1-ylmethyl)-3-trifluoromethyl-phenyl]-urea

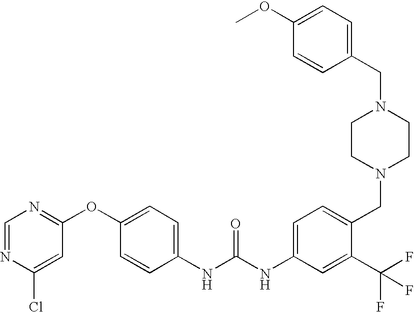

Prepared in analogy to Ex. 14 starting from 878 mg (2.3 mMol) of 4-[4-(4-methoxy-benzyl)-piperazinyl]-3-3-trifluoromethyl-phenylamine and 573 mg (2.3 mMol) 4-chloro-6-(4-isocyanato-phenoxy)-pyrimidine (Step 1.3) to give the title compound: MS (ES+), M+H=628. ¹H-NMR (300 MHz, CDCl₃): 8.59 (s, 1H), 7.75 (d, 1H), 7.41 (d, 2H), 7.20 (d, 2H), 7.17 (d, 2H), 6.98 (s, 1H), 6.83 (d, 3H), 6.79 (s, 1H), 3.80 (s, 3H), 3.59 (s, 2H), 3.42 (s, 2H), 2.58-2.37 (m, 8H).

### Example 21

### N-[4-(6-Chloro-pyrimidin-4-yloxy)-phenyl]-N′-[4-(methyl-^{tert}butyl-amino-methyl)-3-trifluoromethyl-phenyl]-urea

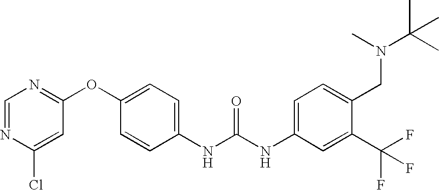

Analogously to Ex. 14, 1.0 g (4.0 mMol) 4-chloro-6-(4-isocyanato-phenoxy)-pyrimidine (Step 1.3) dissolved in 3 ml THF and a solution of 1.1 g (4.2 mMol) of 4-(methyl-^{tert}butyl-amino-methyl-3-trifluoromethyl-aniline (Step 21.2) in 30 ml ether are reacted to the title compound: Anal. C₂₄H₂₅N₅ClF₃O₂: C, H, N, Cl, F; MS: [M+1]⁺=508; ¹H-NMR (CDCl₃): 8.61 (s, 1H), 7.94 (d, 8.2 Hz, 1H). 7.63 (d, 2 Hz, 1H), 7.54 (dd, 8 Hz, 2 Hz, 1H), 7.47 (d, 9 Hz, 2H), 7.14 (d, 9 Hz, 2H), 6.95 (s, 1H), 6.93 (s, 1H), 6.91 (s, 1H), 3.69 (s, 2H), 2.13 (s, H₃C), 1.17 (s, ^{tert}butyl).

The starting material is prepared as follows:

### Step 21.1: 2,2,2-Trifluoro-N-[4-(methyl-^{tert}butyl-amino-methyl)-3-trifluoromethyl-phenyl]-acetamide

To an ice-cooled solution of 2.05 ml (17 mMol) methyl-^{tert}butyl-amine in 80 ml acetonitrile under N₂-atmosphere, a solution of 2.0 g (5.7 mMol) N-(4-bromomethyl-3-trifluoromethylphenyl)-2,2,2-trifluoro-acetamide (Step 14.2) in 80 ml acetonitrile is added dropwise during 30 min. After additional 30 min, a workup procedure as described in Step 14.3 gives the title compound as an oil: MS: [M+1]⁺=357; HPLC ^{A}t_{Ret}=10.0.

### Step 21.2: 4-(Methyl-^{tert}butyl-amino-methyl)-3-trifluoromethyl-aniline

Saponification of 2.55 g (7.2 mMol) of 2,2,2-trifluoro-N-[4-(methyl-^{tert}butyl-amino-methyl)-3-trifluoromethyl-phenyl]-acetamide as described in Step 15.2 gives the title compound as an oil: MS: [M+1]⁺=261; HPLC ^{A}t_{Ret}=8.3.

### Example 22

### N-[4-(6-Chloro-pyrimidin-4-yloxy)-phenyl]-N′-[4-(azetidin-1-ylmethyl)-3-trifluoromethyl-phenyl]-urea

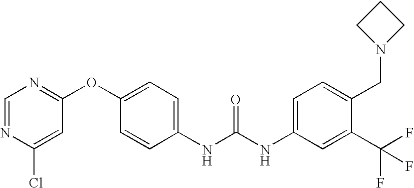

Analogously to Ex. 14, 431 mg (1.7 mMol) 4-chloro-6-(4-isocyanato-phenoxy)-pyrimidine (Step 1.3) dissolved in 2 ml THF and a solution of 400 mg (1.7 mMol) of 4-(azetidin-1-ylmethyl)-3-trifluoromethyl-aniline (Step 22.2) in 10 ml ether are reacted to the title compound: MS: [M+1]⁺=478; HPLC ^{A}t_{Ret}=11.3.

The starting material is prepared as follows:

### Step 22.1: 2,2,2-Trifluoro-N-[4-(azetidin-1-ylmethyl)-3-trifluoromethyl-phenyl]-acetamide

To an ice-cooled solution of 1.74 ml (25.7 mMol) azedidine in 100 ml acetonitrile under N₂-atmosphere, a solution of 3.0 g (8.5 mMol) N-(4-bromomethyl-3-trifluoromethyl-phenyl)-2,2,2-trifluoro-acetamide (Step 14.2) in 100 ml acetonitrile is added dropwise during 65 min. After additional 75 min, a workup procedure as described in Step 14.3 gives the title compound as an oil: MS: [M+1]⁺=327; HPLC ^{A}t_{Ret}=0.1.

### Step 22.2: 4-(Azetidin-1-ylmethyl)-3-trifluoromethyl-aniline

Saponification of 2.67 g (8.2 mMol) of 2,2,2-trifluoro-N-[4-(azetidin-1-ylmethyl)-3-trifluoromethyl-phenyl]-acetamide as described in Step 15.2 gives the title compound as an oil: MS: [M+1]⁺=231; ¹H-NMR (CDCl₃): 7.37 (d, 8.2 Hz, 1H), 6.90 (d, 2 Hz, 1H), 6.79 (dd, 8 Hz, 2 Hz, 1H), 3.75 (s, H₂N), 3.64 (s, 2H), 3.25 (t, 6.8 Hz, 4H), 2.10 (quint, 6.8 Hz, 2H).

### Example 23

### N-[4-(6-Chloro-pyrimidin-4-yloxy)-phenyl]-N′-[4-(4,5-dimethylimidazol-1-ylmethyl)-3-trifluoromethyl-phenyl]-urea

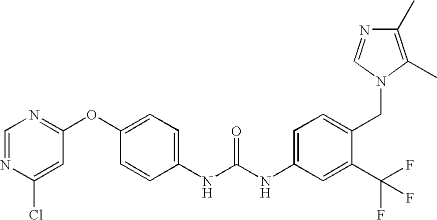

238 mg (0.96 mMol) 4-chloro-6-(4-isocyanato-phenoxy)-pyrimidine (Step 1.3) and 246 mg (0.91 mMol) of 4-(4,5-dimethylimidazol-1-ylmethyl)-3-trifluoromethyl-aniline (Step 23.2) are dissolved in 5 ml THF under N₂-atmosphere. After 15 min, 10 ml of DIPE are added (precipitation formed) and stirring continued for 2 h. Filtration and washing with DIPE gives the title compound: m.p.: 195-196° C.; Anal. C₂₄H₂₀N₆ClF₃O₂.0.4 DIPE.0.1 THF: C, H, N, Cl, F; MS: [M+1]⁺=517; ¹H-NMR (CDCl_{.}): 9.23 (s, 1H), 8.99 (s, 1H), 8.52 (s, 1H), 8.46 (d, 2 Hz, 1H), 7.55 (d, 9.0 Hz, 2H), 7.45 (s, 1H), 7.03 (d, 9 Hz, 2H), 6.83 (s, 1H), 6.34 (dd, 8.6 Hz, 2 Hz, 1H), 6.12 (d, 8.6 Hz, 1H), 5.15 (s, 2H), 2.20 (s, H₃C), 2.02 (s, H₃C).

The starting material is prepared as follows:

### Step 23.1: 2,2,2-Trifluoro-N-[4-(4,5-dimethylimidazol-1-ylmethyl)-3-trifluoromethyl-phenyl]-acetamide

To an ice-cooled solution of 1.81 g (18.8 mMol) 4,5-dimethylimidazol in 70 ml acetonitrile under N₂-atmosphere, a solution of 2.2 g (6.3 mMol) N-(4-bromomethyl-3-trifluoromethylphenyl)-2,2,2-trifluoro-acetamide (Step 14.2) in 70 ml acetonitrile is added dropwise during 30 min. After 5 h, the suspension is filtered and the residue washed with CH₃CN, yielding the title compound (more product can be isolated from the filtrate by concentration and extraction as described in Step 14.3): m.p.: 238-239° C.; MS: [M+1]⁺=366.

### Step 23.2: 4-(4,5-Dimethylimidazol-1-ylmethyl)-3-trifluoromethyl-aniline

Saponification of 2.67 g (7.3 mMol) of 2,2,2-trifluoro-N-[4-(4,5-dimethylimidazol-1-ylmethyl)-3-trifluoromethyl-phenyl]-acetamide as described in Step 15.2 gives upon chromatography (SiO₂: EtOAC/Et₃N 99:1→EtOAC/EtOH/Et₃N 97:2:1) and crystallization from EtOAc the title compound: m.p.: 185-186° C.; MS: [M+1]⁺=270.

### Example 24

### N-[4-(6-Chloro-pyrimidin-4-yloxy)-phenyl]-N′-[4-(2-methylimidazol-1-ylmethyl)-3-trifluoromethyl-phenyl]-urea

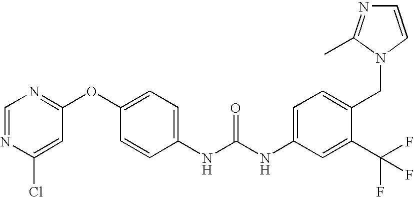

1.00 g (4.04 mMol) 4-chloro-6-(4-isocyanato-phenoxy)-pyrimidine (Step 1.3) and 1.03 g (4.04 mMol) of 4-(2-methylimidazol-1-ylmethyl)-3-trifluoromethyl-aniline (Step 24.2) are dissolved in 40 ml THF under N₂-atmosphere. During stirring at rt for 4 h, a suspension is formed and the title compound can be filtered off: m.p.: 228° C.; Anal. C₂₃H₁₈N₆ClF₃O₂: C, H, N, Cl; MS: [M+1]⁺=503; ¹H-NMR (DMSO-d₆): 9.15 (s, 1H), 8.93 (s, 1H), 8.67 (s, 1H), 8.14 (d, 2 Hz, 1H), 7.55 (d, 9.0 Hz, 2H), 7.54 (m, 1H), 7.36 (s, 1H), 7.19 (d, 9 Hz, 2H), 7.08 (s, 1H), 6.84 (s, 1H), 6.66 (d, 8.6 Hz, 1H), 5.27 (s, 2H), 2.20 (s, H₃C).

The starting material is prepared as follows:

### Step 24.1: 2,2,2-Trifluoro-N-[4-(2-methylimidazol-1-ylmethyl)-3-trifluoromethyl-phenyl]-acetamide

To an ice-cooled suspension of 1.85 g (22.5 mMol) 2-methylimidazol in 80 ml acetonitrile under N₂-atmosphere, a solution of 2.64 g (7.5 mMol) N-(4-bromomethyl-3-trifluoromethyl-phenyl)-2,2,2-trifluoro-acetamide (Step 14.2) in 80 ml acetonitrile is added dropwise during 30 min. Upon stirring for 5 h at rt, a solution is formed, which then is concentrated in vacuo. The residue is diluted with EtOAc and saturated NaHCO₃-solution/H₂O 1:1. The aqueous layer is separated off and extracted twice with EtOAc. The organic layers are washed with saturated NaHCO₃-solution/H₂O 1:1, water and brine, dried (Na₂SO₄) and concentrated. Column chromatography (SiO₂: EtOAC/EtOH 19:1→9:1) gives the title compound: m.p.: 229-230° C.; MS: [M+1]⁺=352.

### Step 24.2: 4-(2-Methylimidazol-1-ylmethyl)-3-trifluoromethyl-aniline

Saponification of 2.0 g (5.69 mMol) of 2,2,2-trifluoro-N-[4-(2-methylimidazol-1-ylmethyl)-3-trifluoromethyl-phenyl]-acetamide as described in Step 15.2 gives after crystallization from EtOAc the title compound: m.p.: 146-147° C.; MS: [M+1]⁺=256.

### Example 25

### N-[4-(6-Chloro-pyrimidin-4-yloxy)-phenyl]-N′-[4-(2,4-dimethylimidazol-1-ylmethyl)-3-trifluoromethyl-phenyl]urea

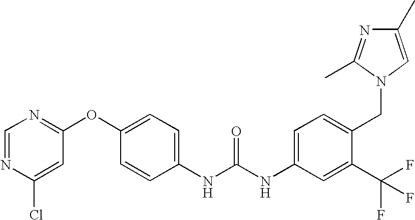

Can be prepared analogously to Ex. 23 or 24.

### Example 26

### N-[4-(6-Chloro-pyrimidin-4-yloxy)-phenyl]-N′-[4-(4-ethylpiperazin-1-ylmethyl)-3-methyl-phenyl]-urea

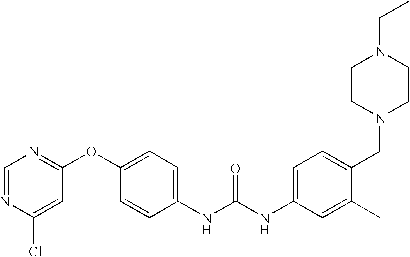

Analogously to Ex. 14, 467 mg (1.88 mMol) 4-chloro-6-(4-isocyanato-phenoxy)-pyrimidine (Step 1.3) dissolved in 2 ml THF and a suspension of 440 mg (1.88 mMol) of 4-(4-ethylpiperazin-1-ylmethyl)-3-methyl-aniline (Step 26.4) in 8 ml ether are reacted to the title compound: MS: [M+1]⁺=481; ¹H-NMR (DMSO-d₆): 8.77 (s, 1H), 8.67 (s, 1H), 8.60 (s, 1H), 7.53 (d, 9.0 Hz, 2H), 7.35 (d, 0.8 Hz, 1H), 7.21-7.27 (m, 2H), 7.17 (d, 9.0 Hz, 2H), 7.10 (d, 8.2 Hz, 1H), 3.34 (s, 2H), 2.36 (m, 10H), 2.30 (s, H₃C), 0.98 (t, 7.2 Hz, H₃C).

The starting material is prepared as follows:

### Step 26.1: 4-Nitro-2-methyl-benzoic acid

A mixture of 3.04 g (18.7 mMol) of 2-methyl-4-nitrobenzonitrile [preparation see: J. Med. Chem. 44 (2001), 3856], 26 ml HCl conc. and 26 ml acetic acid is heated in a sealed tube for 8 h to 150° C. Filtration of the cool reaction mixture and washing with water gives the title compound: m.p.: 151-155° C.; MS: [M−1]⁺=180.

### Step 26.2: (4-Nitro-2-methyl-phenyl)-(4-ethylpiperazin-1-yl)-methanone

Analogously to Step 5.1, 8.72 g (48.1 mMol) of 4-nitro-2-methyl-benzoic acid are activated with 6.52 ml (77 mmol) of oxalylchloride and reacted with 13.45 ml (106 mMol) of 1-ethylpiperazine, yielding the title compound: m.p.: 96-99° C.; MS: [M+1]⁺=278.

### Step 26.3: (4-Amino-2-methyl-phenyl)-(4-ethylpiperazin-1-yl)-methanone

Hydrogenation of 12.6 g (45.5 mMol) of (4-nitro-2-methyl-phenyl)-(4-ethylpiperazin-1-yl)-methanone in 200 ml ethanol in the presence of 2 g of Raney-Nickel as described in Step 1.5 gives the title compound as an oil: MS: [M+1]⁺=248.

### Step 26.4: 4-(4-Ethylpiperazin-1-ylmethyl)-3-methyl-aniline

Analogously to Step 5.3, 11.12 g (45 mMol) (4-amino-2-methyl-phenyl)-(4-ethylpiperazin-1-yl)-methanone in 100 ml THF are reduced by 135 ml BH₃ (1M in THF). Chromatography (SiO₂; CH₂Cl₂/MeOH/NH₃^{aq} 97:3:1) gives the oily title compound: MS: [M+1]⁺=234; ¹H-NMR (CDCl₃): 7.04 (d, 8.2 Hz, 1H), 6.54 (d, 2.4 Hz, 1H), 6.51 (dd, 8 Hz, 2.4 Hz, 1H), 3.59 (s, H₂N), 3.39 (s, 2H), 2.5 (m, 8H), 2.43 (q, 7.2 Hz, 2H), 2.31 (s, H₃C), 1.11 (t, 7.2 Hz, H₃C).

### Example 27

### N-[4-(6-Chloro-pyrimidin-4-yloxy)-phenyl]-N′-[4-(4-ethylpiperazin-1-yl methyl)-phenyl]-urea

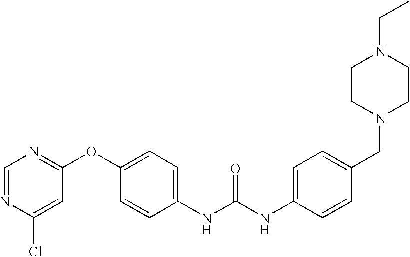

A solution of 230 mg (0.93 mMol) 4-chloro-6-(4-isocyanato-phenoxy)-pyrimidine (Step 1.3) and 200 mg (0.91 mMol) of 4-(4-ethylpiperazin-1-ylmethyl)-aniline in 8 ml THF is stirred for 40 min at rt. Crystallization by addition of ≈15 ml of DIPE, filtration and washing with DIPE gives the title compound: m.p.: 203-204° C.; MS: [M+1]⁺=467; ¹H-NMR (CDCl₃): 8.62 (s, 1H), 7.48 (d, 9.0 Hz, 2H), 7.33 (m, 4H), 7.13 (d, 9.0 Hz, 2H), 6.95 (s, 1H), 6.88 (s, 1H), 6.75 (s, 1H), 3.52 (s, 2H), 2.53 (m, 8H), 2.45 (q, 7.0 Hz, 2H), 1.12 (t, 7.0 Hz, H₃C).

The starting material is prepared as follows:

### Step 27.1: 4-(4-Ethylpiperazin-1-ylmethyl)-aniline

Analogously to Step 5.3, 7.8 g (33.4 mMol) (4-aminophenyl)-(4-ethylpiperazin-1-yl)-methanone [synthesis as described above or alternatively in J. Pharmaceutical Sci. 57 (1968), 2073] in 105 ml THF are reduced by 100 ml BH₃ (1M in THF) at 65° C.: MS: [M+1]⁺=220; ¹H-NMR (CDCl₃): 7.13 (d, 8.2 Hz, 2H), 6.68 (d, 8.2 Hz, 2H), 3.67 (s, H₂N), 3.47 (s, 2H), 2.6 (m, 8H), 2.53 (q, 7.3 Hz, 2H), 1.16 (t, 7.3 Hz, H₃C).

### Example 28

### 1-(4-[1,4′]Bipiperidinyl-1′-yl-3-trifluoromethyl-phenyl)-3-[4-(6-chloro-pyrimidin-4-yloxy)-phenyl]-urea

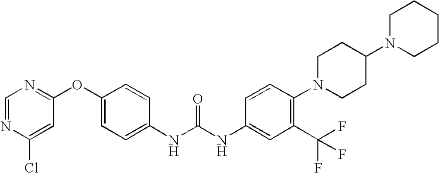

A solution of 248 mg (1.0 mMol) 4-chloro-6-(4-isocyanato-phenoxy)-pyrimidine (Step 1.3) and 327 mg (1.0 mMol) of 4-[1,4′]bipiperidinyl-1′-yl-3-trifluoromethyl-phenylamine (Step 28.2) in 8 ml THF is stirred for 30 min at rt. Crystallization by addition of =15 ml of DIPE, filtration and washing with DIPE gives the title compound: MS: [M+1]⁺=575; HPLC ^{B}t_{Ret}=2.06.

The starting material is prepared as follows:

### Step 28.1: 1′-(4-Nitro-2-trifluoromethyl-phenyl)-[1,4′]bipiperidinyl

A solution of 1.0 mL (7.27 mMol) of 1-fluoro-4-nitro-2-trifluoromethyl-benzene, 1.47 g (8.73 mMol) [1,4′]bipiperidinyl and 1.51 g (10.9 mMol) K₂CO₃ in 15 ml DMF is stirred at room temperature for 17 h. After evaporating the DMF under reduced pressure, the reaction mixture is diluted with 80 ml H₂O and extracted 3× with 60 ml of EtOAc. The combined organic phases are washed with 30 ml H₂O and 30 ml brine, dried (MgSO₄), concentrated under reduced pressure and flash chromatographed (SiO₂; 4.0×24 cm, MeOH/CH₂Cl₂ 1:19) to give the title compound as oil: ¹H-NMR (400 MHz, CDCl₃): 8.45 (dd, 1H), 8.25 (dd, 1H), 7.20 (dd, 1H), 3.45 (m, 2H), 2.88 (m, 2H), 2.58 (m, 4H), 2.40 (m, 1H), 1.60 (m, 10H).

### Step 28.2: 4-[1,4′]Bipiperidinyl-1′-yl-3-trifluoromethyl-phenylamine

Hydrogenation of 2.14 g (5.99 mMol) of 1′-(4-nitro-2-trifluoromethyl-phenyl)-[1,4′]bipiperidinyl in 25 ml ethanol in the presence of 220 mg of 10% Pd/C as described in Step 1.5 gives the title compound as an oil: MS: [M+1]⁺=32a.

### Example 29

### 1-[4-(6-Chloro-pyrimidin-4-yloxy)-phenyl]-3-{4-[4-(2,2-dimethyl-propyl)-piperazin-1-ylmethyl]-3-trifluoromethyl-phenyl}-urea

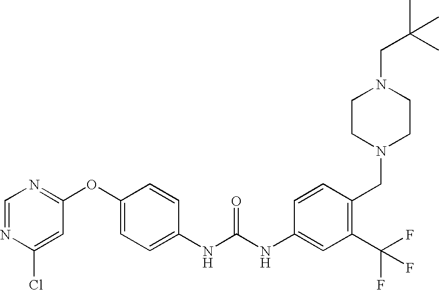

A solution of 112 mg (0.45 mMol) 4-chloro-6-(4-isocyanato-phenoxy)-pyrimidine (Step 1.3) and 150 mg (0.45 mMol) of 4-[4-(2,2-dimethyl-propyl)-piperazin-1-ylmethyl]-3-trifluoromethyl-phenylamine (Step 29.4) in 8 ml THF is stirred for 30 min at rt. Crystallization by addition of ≈15 ml of DIPE, filtration and washing with DIPE gives the title compound: MS: [M+1]⁺=578; HPLC ^{B}t_{Ret}=2.18; ¹H-NMR (de-DIMSO): 9.00 (bs, 1H), 8.82 (bs, 1H), 8.60 (s, 1H), 7.94 (s, 1H), 7.5 (m, 4H), 7.30 (s, 1H), 7.10 (m, 2H), 3.46 (bs, 2H), 2.45 (m, 4H), 2.35 (m, 4H), 2.00 (s, 2H), 0.80 (s, 9H).

The starting material is prepared as follows:

### Step 29.1: 3-[2-(2,2-Dimethyl-proyylamino)-ethyl]-oxazolidin-2-one

A solution of 5 g (17.5 mMol) of toluene-4-sulfonic acid 2-(2-oxo-oxazolidin-3-yl)-ethyl ester, 1.68 g (19.2 mMol) 2,2-dimethyl-propylamine and 3.63 g (26.3 mMol) K₂CO₃ in 35 ml MeCN is stirred at 40° C. for 12 h. After evaporating the MeOH under reduced pressure, the reaction mixture is diluted with 80 ml H₂O and extracted 3× with 60 ml of EtOAc. The combined organic phases are washed with 30 ml H₂O and 30 ml brine, dried (MgSO₄) and concentrated under reduced pressure to give the title crude compound as oil. MS: [M+1]⁺=201; ¹H-NMR (CDCl₃): 4.30 (dd, 2H), 3.65 (dd, 2H), 3.35 (t, 2H), 2.80 (t, 2H), 2.35 (s, 2H), 0.90 (s, 9H).

### Step 29.2: 1-(2,2-Dimethyl-propyl)-piperazine dihydrobromide salt

1-(2,2-Dimethyl-propyl)-piperazine dihydrobromide salt is prepared using 3-[2-(2,2-dimethyl-propylamino)-ethyl]oxazolidin-2-one according to a literature procedure (Tetrahedron Letters, 40, 7331, 1994): MS: [M+1]⁺=157.

### Step 29.3: N-{4-[4-(2,2-Dimethyl-propyl)-piperazin-1-ylmethyl]-3-trifluoromethyl-phenyl}-2,2,2-trifluoro-acetamide

1.0 g (3.14 mMol) 1-(2,2-dimethyl-propyl)-piperazine dihydrobromide salt, 440 mg (1.25 mMol) N-(4-bromomethyl-3-trifluoromethyl-phenyl)-2,2,2-trifluoro-acetamide (Step 14.2), and 0.53 mL (3.77 mMol) triethylamine, dissolved in 10 ml DMF are stirred for 3 h at rt. After evaporating the acetonitrile under reduced pressure, the reaction mixture is diluted with 80 ml H₂O and extracted 3 times with 70 ml of EtOAc. The combined organic phases are washed twice with 30 ml NaHCO₃ solution and 30 ml brine, dried (MgSO₄), concentrated under reduced pressure and flash chromatographed (MeOH/CH₂Cl₂ 1:19), to give a yellow solid: MS: [M+1]⁺=426; HPLC ^{B}t_{Ret}=2.13.

### Step 29.4: 4-[4-(2,2-Dimethyl-propyl)-piperazin-1-ylmethyl]-3-trifluoromethyl-phenylamine

To a solution of 445 mg (1.04 mMol) of N-{4-[4-(2,2-dimethyl-propyl)-piperazin-1-ylmethyl]-3-trifluoromethyl-phenyl}-2,2,2-trifluoro-acetamide in 18 ml of boiling methanol, 5.2 ml of a 1 M solution of K₂CO₃ in water are added dropwise. After 1 h stirring, the reaction mixture is cooled to rt and diluted with EtOAc and water. The aqueous layer is separated off and extracted twice with EtOAc. The organic phases are washed with water and brine, dried (Na₂SO₄) and concentrated to yield the title compound, which is directly used in Ex. 29: MS: [M+1]⁺=330; HPLC ^{D}t_{Ret}=1.73.

### Example 30

### 1-[4-6-Chloro-pyrimidin-4-yloxy)-phenyl]-3-{4-[4-(2,2-dimethyl-propyl)-piperazin-1-yl]-3-trifluoromethyl-phenyl}-urea

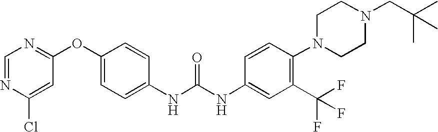

A solution of 141 mg (0.57 mMol) 4-chloro-6-(4-isocyanato-phenoxy)-pyrimidine (Step 1.3) and 180 mg (0.57 mMol) of 4-[4-(2,2-dimethyl-propyl)-piperazin-1-yl]-3-trifluoromethyl-phenylamine (Step 30.2) in 8 ml THF is stirred for 30 min at rt. Crystallization by addition of ≈15 ml of DIPE, filtration and washing with DIPE gives the title compound: MS: [M+1]⁺=563 HPLC ^{D}t_{Ret}=2.28.

The starting material is prepared as follows:

### Step 30.1: 1-(2,2-Dimethyl-propyl)-4-(4-nitro-2-trifluoromethyl-phenyl)-piperazine

A solution of 0.36 mL (2.62 mMol) of 1-fluoro-4-nitro-2-trifluoromethyl-benzene, 1.0 g (3.14 mMol) 1-(2,2-dimethyl-propyl)-piperazine dihydrobromide salt and 1.08 g (7.86 mMol) K₂CO₃ in 8 ml DMF is stirred at room temperature for 17 h. After evaporating the DMF under reduced pressure, the reaction mixture is diluted with 80 ml H₂O and extracted 3× with 60 ml of EtOAc. The combined organic phases are washed with 30 ml H₂O and 30 ml brine, dried (MgSO₄), concentrated under reduced pressure and flash chromatographed (SiO₂, MeOH/CH₂Cl₂ 1:19) to give the title compound as oil: MS: [M+1]⁺=346; HPLC ^{D}t_{Ret}=2.39; ¹H-NMR (300 MHz, CDCl₃): 8.50 (dd, 1H), 8.30 (dd, 1H), 7.25 (dd, 1H), 3.15 (m, 4H), 2.70 (m, 4H), 2.10 (s, 2H), 0.90 (s, 9H).

### Step 30.2: 4-[4-(2,2-Dimethyl-propyl)-piperazin-1-yl]-3-trifluoromethyl-phenylamine

Hydrogenation of 210 mg (0.63 mMol) of 1-(2,2-Dimethyl-propyl)-4-(4-nitro-2-trifluoromethyl-phenyl)-piperazine in 10 ml ethanol in the presence of 40 mg of 10% Pd/C as described in Step 1.5 gives the title compound as an oil: MS: [M+1]⁺=316.

### Example 31

### 1-[4-(6-Chloro-pyrimidin-4-yloxy)-phenyl]-3-[4-(1-methyl-piperidin-4-ylmethoxy)-3-trifluoromethyl-phenyl]-urea

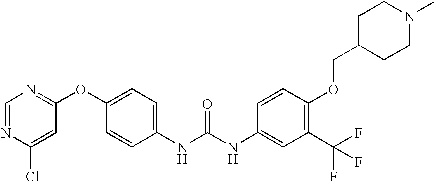

A solution of 248 mg (1.00 mMol) 4-chloro-6-(4-isocyanato-phenoxy)-pyrimidine (Step 1.3) and 288 mg (1.00 mMol) of 4-(1-methyl-piperidin-4-ylmethoxy)-3-trifluoromethyl-phenylamine (Step 31.2) in 8 ml THF is stirred for 30 min at rt. Crystallization by addition of ≈15 ml of DIPE, filtration and washing with DIPE gives the title compound: MS: [M+1]⁺=535; HPLC ^{A}t_{Ret}=1.98.

The starting material is prepared as follows:

### Step 31.1: 1-Methyl-4-(4-nitro-2-trifluoromethyl-phenoxymethyl)-piperidine

A solution of 1.00 mL (7.27 mMol) of 1-fluoro-4-nitro-2-trifluoromethyl-benzene, 1.88 g (14.5 mMol) (1-methyl-piperidin-4-yl)-methanol and 470 mg (1.45 mMol) tetrabutylammonium bromide in 6 ml toluene and 6 ml 25% KOH_{aq} is stirred at 60° C. for 17 h. After cooling the solution, the reaction mixture is diluted with 80 ml H₂O and extracted 3× with 60 ml of EtOAc. The combined organic phases are washed twice with 30 ml NaHCO₃ solution and 30 ml brine, dried (MgSO₄), concentrated under reduced pressure and flash chromatographed (MeOH/CH₂Cl₂ 1:19) to give the title compound: MS: [M+1]⁺=319.

### Step 31.2: 4-(1-Methyl-piperidin-4-ylmethoxy)-3-trifluoromethyl-phenylamine

Hydrogenation of 1.86 g (5.84 mMol) of 1-methyl-4-(4-nitro-2-trifluoromethyl-phenoxymethyl)-piperidine in 20 ml ethanol in the presence of 190 mg of 10% Pd/C as described in Step 1.5 gives the title compound as an oil: MS: [M+1]⁺=289.

### Example 32

### 1-[4-(6-Chloro-pyrimidin-4-yloxy)-phenyl]-3-[4-(1-methyl-piperidin-4-yloxy)-3-trifluoromethyl-phenyl]-urea

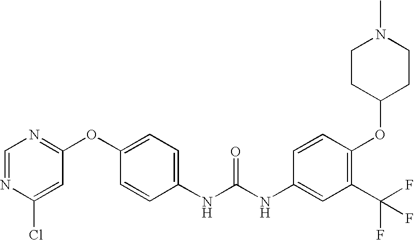

A solution of 248 mg (1.00 mMol) 4-chloro-6-(4-isocyanato-phenoxy)-pyrimidine (Step 1.3) and 274 mg (1.00 mMol) of 4-(1-methyl-piperidin-4-yloxy)-3-trifluoromethyl-phenylamine (Step 32.2) in 8 ml THF is stirred for 30 min at rt. Crystallization by addition of ≈15 ml of DIPE, filtration and washing with DIPE gives the title compound: MS: [M+1]⁺=522; HPLC ^{A}t_{Ret}=1.96.

The starting material is prepared as follows:

### Step 32.1: 1-Methyl-4-(4-nitro-2-trifluoromethyl-phenoxy)-piperidine

A solution of 1.00 mL (7.27 mMol) of 1-fluoro-4-nitro-2-trifluoromethyl-benzene, 1.71 ml (14.5 mMol) 1-methyl-piperidin-4-ol and 470 mg (1.45 mMol) tetrabutylammonium bromide in 6 ml toluene and 6 ml 25% KOH_{aq} is stirred at 60° C. for 17 h. After cooling the solution, the reaction mixture is diluted with 80 ml H₂O and extracted 3× with 60 ml of EtOAc. The combined organic phases are washed twice with 30 ml NaHCO₃ solution and 30 ml brine, dried (MgSO₄), concentrated under reduced pressure and flash chromatographed (MeOH/CH₂Cl₂ 1:19) to give the title compound: MS: [M+1]⁺=305.

### Step 32.2: 4-(1-Methyl-piperidin-4-yloxy)-3-trifluoromethyl-phenylamine

Hydrogenation of 1.74 g (5.72 mMol) of 1-methyl-4-(4-nitro-2-trifluoromethyl-phenoxy)-piperidine in 20 ml ethanol in the presence of 180 mg of 10% Pd/C as described in Step 1.5 gives the title compound as an oil: MS: [M+1]⁺=275.

### Example 33

### N-[4-(6-Chloro-pyrimidin-4-yloxy)-phenyl]-N′-{4-[2-(4-ethyl-piperazin-1-yl)-ethyl]-3-trifluoromethyl-phenyl}-urea

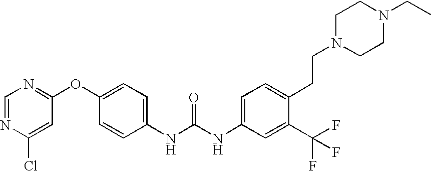

370 mg (1.49 mMol) 4-chloro-6-(4-isocyanato-phenoxy)-pyrimidine (Step 1.3) and 450 mg (1.49 mMol) of 4-[2-(4-ethyl-piperazin-1-yl)-ethyl]-3-trifluoromethyl-phenylamine (Step 33.3) are dissolved in 1.4 ml THF and 7.4 ml ether under N₂-atmosphere and stirred for 1 h. Concentration and reversed phase chromatography (Gilson System) gives the title compound: HPLC ^{A}t_{Ref}=11.1; MS: [M+1]⁺=549; ¹H-NMR (CDCl₃): 8.60 (s, 1H), 7.58 (d, 1H), 7.57 (s, 1H), 7.46 (d, 9.0 Hz, 2H), 7.30 (m, 1H), 7.12 (m, 4H), 6.95 (s, 1H), 2.94 (m, 2H), 2.6 (m, 12H), 1.13 (t, 7.2 Hz, H₃C).

The starting material is prepared as follows:

### Step 33.1: 2-(4-Nitro-2-trifluoromethyl-phenyl)-1-(4-ethyl-piperazin-1-yl)-ethanone

To an ice-cooled solution of 11.4 g (45.9 mMol) (4-nitro-2-trifluoromethyl-phenyl)-acetic acid in 200 ml CH₂Cl₂ and 2 ml DMF, 7.36 ml (87.2 mMol) oxalylchloride are added dropwise. After 20 min the reaction mixture is concentrated in vacuo. The residue is re-dissolved in 200 ml CH₂Cl₂ and a solution of 12.2 ml (96 mMol) N-ethyl-piperazine in 80 ml CH₂Cl₂ is added dropwise. After 1 h the mixture is diluted with 0.41 of a 10% solution of Na₂CO₃ and 0.4 l CH₂Cl₂, the aqueous layer separated off and extracted twice with CH₂Cl₂. Washing of the organic phases twice with a 10% solution of Na₂CO₃, water and brine, drying (Na₂SO₄) and concentration gives the title compound: HPLC ^{A}t_{Ret}=9.2; MS: [M+1]⁺=346.

### Step 33.2: 2-(4-Amino-2-trifluoromethyl-phenyl)-1-(4-ethyl-piperazin-1-yl)-ethanone

15.35 g (44.5 mMol) 2-(4-nitro-2-trifluoromethyl-phenyl)-1-(4-ethyl-piperazin-1-yl)-ethanone in 245 ml ethanol are hydrogenated in presence of 2.46 g Raney Nickel (B113W Degussa). Filtration, concentration of the filtrate and column chromatography (SiO₂; EtOAc/EtOH+1% NH₃^{aq}4:1) gives the title compound: MS: [M+1]⁺=316; R^{f}(EtOAc/EtOH+1% NH₃^{aq} 4:1): 0.11.

### Step 33.3: 4-[2-(4-Ethyl-piperazin-1-yl)-ethyl]-3-trifluoromethyl-phenylamine

To a solution of 3.47 g (11.0 mMol) 2-(4-amino-2-trifluoromethyl-phenyl)-1-(4-ethyl-piperazin-1-yl)-ethanone in 35 ml THF, 46.8 ml of a 1 M solution of BH₃ in THF are added dropwise during 30 min. After stirring for 20 h, 60 ml of a 1:1-mixture of HCl conc. and water are added dropwise during 20 min at 30° C. The mixture is stirred for 16 h at rt and then partially concentrated in vacuo. The residue is extracted 3 times with EtOAc and the organic layers washed with 0.1 N HCl and then discarded The acidic aqueous phases are made basic by addition of saturated Na₂CO₃ solution and extracted 3 times with EtOAc. The organic layers are washed with brine, dried (Na₂SO₄) and concentrated. Combi Flash chromatography (CH₂Cl₂/MeOH+1% NH₃^{aq} 99:1→95:5) gives the title compound: MS: [M+1]⁺=302.

### Example 34

### The Following Compounds can be Prepared Analogously to the Described Procedures

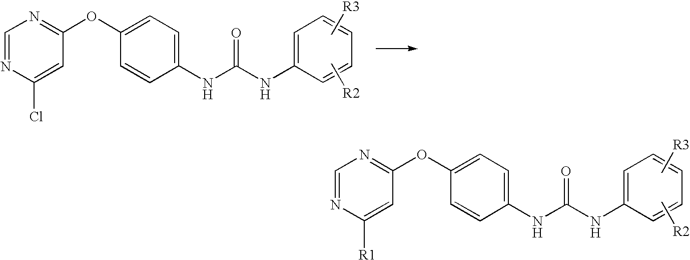

| | 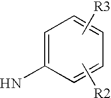 | R1 | HPLC^{A}t_{Ret}[min] | m.p. [° C.] | MS[M + 1]⁺ | Anal. |
|---|---|---|---|---|---|---|
| a.1)a.2)a.3) | 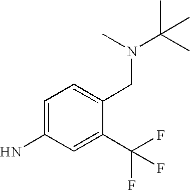 | NH—CH₃N═N⁺═N⁻NH₂ | 9.412.4 8.9 | | 503515489 | CHNF |
| b.1)b.2)b.3) | 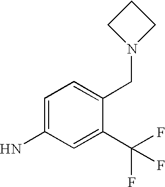 | NH—CH₃N═N⁺═N⁻NH₂ | 8.411.5 8.1 | | 473485459 | |
| c.1)c.2)c.3) | 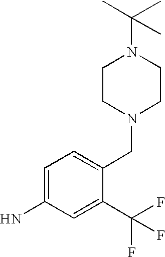 | NH—CH₃N═N⁺═N⁻NH₂ | 2.00^{$}) 2.14^{$}) 1.73^{$}) | 96-98 176-179 | 571545 | |
| d.1)d.2)d.3) | 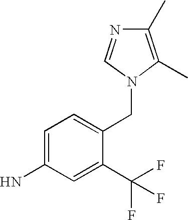 | NH—CH₃N═N⁺═N⁻NH₂ | 9.312.2 9.1 | 251-252 236-237 | 512524498 | CHNF CHNF |
| e.1)e.2)e.3) | 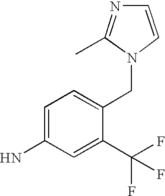 | NH—CH₃N═N⁺═N⁻NH₂ | 8.611.6 8.4 | 248-249 237 | 498510484 | CHNF CHN |
| f.1)f.2)f.3) | 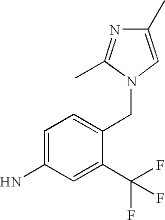 | NH—CH₃N═N⁺═N⁻NH₂ | | | | |
| g.1)g.2)g.3)g.4) | 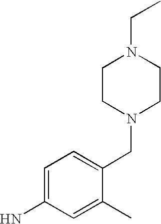 | NH—CH₃N═N⁺═N⁻NH₂NH—C₂H₅**) | 6.5 9.5 6.3 6.9 | | 476488462490 | |
| h.1)h.2)h.3) | 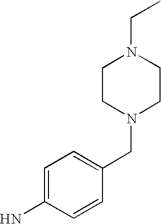 | NH—CH₃N═N⁺═N⁻NH₂ | 6.1 9.2 9.2 | 222-223 | 462474448 | |
| i.1)i.2)i.3) | 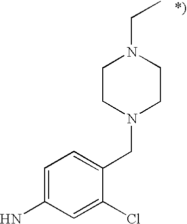 | NH—CH₃N═N⁺═N⁻NH₂ | 7.010.1 6.8 | | 496508482 | |
| j.1)j.2)j.3) | 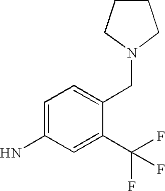 | NH—CH₃N═N⁺═N⁻NH₂ | 2.19^{$}) 1.33^{$}) | 112 56-58 | 487 473 | |
| k.1)k.2)k.3) | 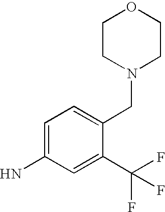 | NH—CH₃N═N⁺═N⁻NH₂ | 1.59^{$}) | 89 89 | 515489 | |
| l.1)l.2)l.3) | 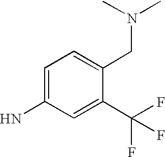 | NH—CH₃N═N⁺═N⁻NH₂ | 2.31^{$}) 2.33^{$}) 2.35^{$}) | 135-137 | 461473447 | |
| m.1)m.2)m.3) | 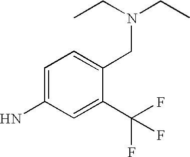 | NH—CH₃N═N⁺═N⁻NH₂ | 2.04^{$}) | 85-86 85-87 | 489501475 | |
| n.1)n.2)n.3) | 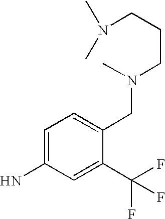 | NH—CH₃N═N⁺═N⁻NH₂ | 1.67^{$}) 2.14^{$}) 1.42^{$}) | 76-78 | 533530518 | |
| o.1)o.2)o.3) | 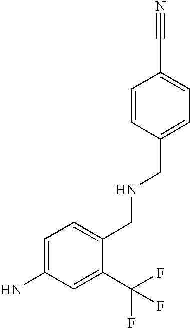 | NH—CH₃N═N⁺═N⁻NH₂ | 2.42^{$}) 1.95^{$}) | 104-107 153-155 | 548560534 | |
| p.1)p.2)p.3) | 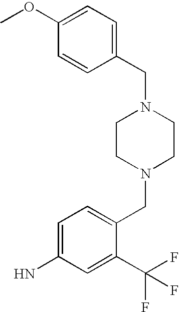 | NH—CH₃N═N⁺═N⁻NH₂ | 1.91^{$}) 2.27^{$}) 1.89^{$}) | 115-117 | 622634608 | |
| q.1)q.2) | 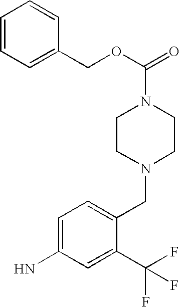 | NH₂N═N⁺═N⁻ | 2.04^{$}) 2.43^{$}) | | 636648 | |
| r.1) r.2)r.3) | 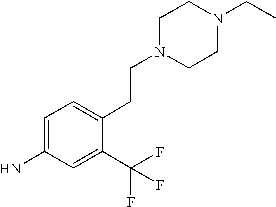 | NH—CH₃**)N═N⁺═N⁻NH₂ | 8.6 7.4 | | 544 556530 | |
| s.1)s.2) | 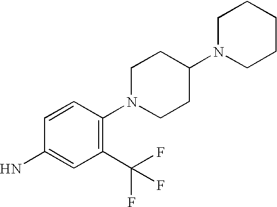 | N═N⁺═N⁻NH₂ | | 158-161 | 581555 | |
| t.1)t.2) | 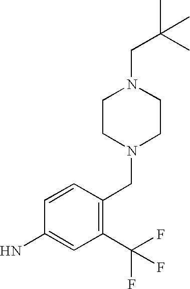 | N═N⁺═N⁻NH₂ | | 154-155 | 584558 | |
| u.1)u.2) | 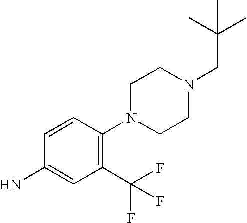 | N═N⁺═N⁻NH₂ | | 150-151 | 570544 | |
| v.1)v.2) | 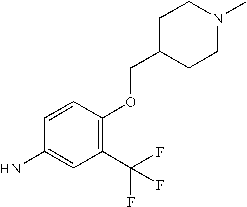 | N═N⁺═N⁻NH₂ | | 151-154 | 542517 | |
| w.1)w.2) | 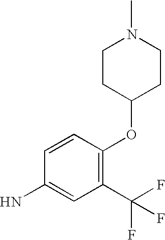 | N═N⁺═N⁻NH₂ | | 147-149 | 528502 | |
| x.1) x.2) | 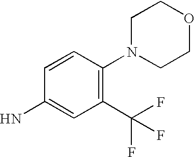 | N═N⁺═N⁻***)NH₂ | 15.4 12.1 | 244-248 | 501 475 | CHNF |

*) Synthesis of educt A see Ex. 65
**) prepared from MeNH₂ respectively EtNH₂ in THF at rt for 4-10 d analogously to Ex. 16.
***) educt Step 69.1
^{$}) ^{D}t_{Ret}

The compounds of Ex. 35-44 can be prepared analogously to the procedures described herein:

### Example 35

### 3-{3-[4-(6-Chloro-pyrimidin-4-yloxy)-phenyl]-ureido}-5-trifluoromethyl-benzamide

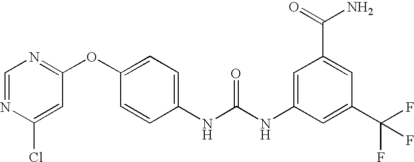

Analogously to Ex. 14, 250 mg (1.0 mMol) 4-chloro-6-(4-isocyanato-phenoxy)-pyrimidine (Step 1.3) dissolved in 2 ml THF and a solution of 204 mg (1.0 mMol) of 3-amino-5-trifluoromethyl)-benzamide (Step 35.2) in 6 ml ether are reacted to the title compound: MS: [M+1]⁺=452; ¹H-NMR (DMSO-d₆): 9.41 (s, 1H, NH), 9.05 (s, 1H, NH), 8.62 (s, 1H), 8.16 (s, 2H, NH2), 8.14 (s, 1H), 8.02 (s, 1H), 7.81 (s, 1H), 7.55-7.52 (m, 3H), 7.32 (s, 1H), 7.17 (d, 2H).

The starting material is prepared as follows:

### Step 35.1°: (3-Nitro-5-trifluoromethyl)-benzamide

Prepared in analogy to Step 1.4 from 2.35 g (10.0 mmol) of 3-nitro-5-trifluoromethyl-benzoic acid (Lancaster), and 20 ml NH₃ (25% aq solution) to give the title compound. MS: [M+1]⁺233.

### Step 35.2: (3-Amino-5-trifluoromethyl)-benzamide

Prepared in analogy to Step 1.5 from 2.34 g (10 mmol) 3-nitro-5-trifluoromethyl)-benzamide by hydrogenation over 250 mg Pd—C (10% Engelhardt 4505). MS: [M+1]⁺=205. ¹H-NMR (400 MHz, DMSO-d₈): 7.99 (s, 1H), 7.31 (s, 1H), 7.19 (s, 2H, NH₂), 6.89 (s, 1H), 5.78 (s, 2H, NH₂). m.p. 94-98° C.

### Example 36

### 3-{3-[4-(6-Methylamino-pyrimidin-4-yloxy)-phenyl]-ureido}-5-trifluoromethyl-benzamide

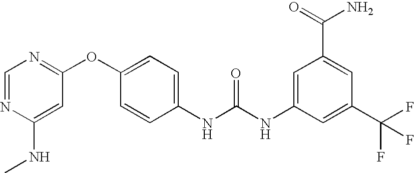

Prepared in analogy to Ex. 16 from 45 mg (0.1 mMol) 3-{3-[4-(6-chloro-pyrimidin-4-yloxy)-phenyl]-ureido}-5-trifluoromethyl-benzamide and 0.8 ml (methylamine (33% in EtOH). MS: [M+1]⁺=447. HPLC ^{B}t_{Ret}: 2.31.

### Example 37

### 3-{3-[4-(6-Azido-pyrimidin-4-yloxy)-phenyl]-ureido}-5-trifluoromethyl-benzamide

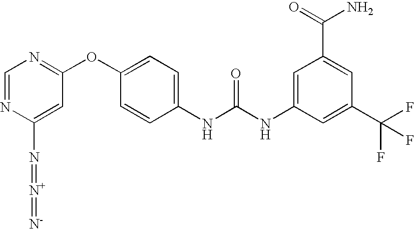

The title compound is prepared from 150 mg (0.33 mMol) of 3-{3-[4-(6-chloro-pyrimidin-4-yloxy)-phenyl]-ureido}-5-trifluoromethyl-benzamide as described in Ex. 7 to yield the title compound which is directly used as starting material in Ex. 38. MS: [M+1]⁺=459.

### Example 38

### 3-{3-[4-(6-Amino-pyrimidin-4-yloxy)-phenyl]-ureido}-5-trifluoromethyl-benzamide

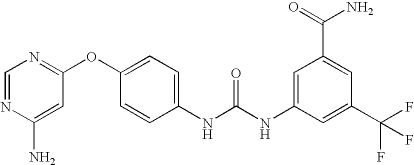

Hydrogenation of 0.15 g (0.33 mMol) of 3-{3-[4-(6-azido-pyrimidin-4-yloxy)-phenyl]-ureido}-5-trifluoromethyl-benzamide in 10 ml DME in the presence of 20 mg Pd/C 10% (“Engelhard 4505”), filtration, concentration of the filtrate and chromatography (Preparatory TLC: CH₂Cl₂/MeOH 9:1) gives the title compound MS: [M+1]⁺=433; ¹H-NMR (DMSO-d₆): 9.72 (s, 1H, NH), 9.43 (s, 1H, NH), 8.18 (s, 1H), 8.16 (s, 1H), 8.06 (s, 2H), 7.78 (s, 1H), 7.52 (d, 2H), 7.05 (d, 2H), 6.82 (s, 2H, NH₂).

### Example 39

### N-[4-(6-Methylamino-pyrimidin-4-yloxy)-phenyl]-N′-(3-aminomethyl-5-trifluoromethyl-phenyl)-urea

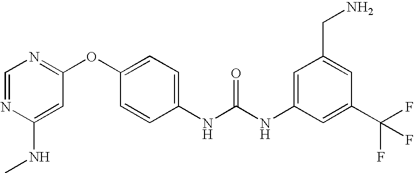

### Example 40

### 3-{3-[4-(6-Chloro-pyrimidin-4-yloxy)-phenyl]-ureido}-N-methyl-5-trifluoromethyl-benzamide

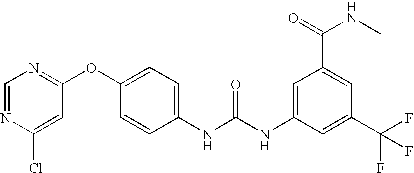

Analogously to Ex. 14, 250 mg (1.5 mMol) 4-chloro-6-(4-isocyanato-phenoxy)-pyrimidine (Step 1.3) dissolved in 3 ml THF and a solution of 218 mg (1.5 mMol) of 3-amino-N-methyl-5-(trifluoromethyl)-benzamide (Step 35.2) in 6 ml ether are reacted to the title compound: MS: [M+1]⁺=466; HPLC ^{B}t_{Ret}: 2.31

The starting material is prepared as follows:

### Step 40.1: N-Methyl (3-nitro-5-trifluoromethyl)-benzamide

Prepared in analogy to Step 1.4 from 2.35 g (10.0 mmol) of 3-nitro-5-trifluoromethyl-benzoic acid (Lancaster), and 40 ml NH₃ (40% aq solution) to give the title compound. MS: [M−1]=247. ¹H-NMR (400 MHz, DMSO-d₆): 9.09 (q, 1H, NH), 8.89 (s, 1H), 8.39 (s, 1H), 8.38 (s, 1H), 2.81 (d, 3H).

### Step 40.2: 3-Amino-N-methyl-5-(trifluoromethyl)-benzamide

Prepared in analogy to Step 1.5 from 2.34 g (10 mmol) N-methyl (3-nitro-5-trifluoromethyl)-benzamide by hydrogenation over 240 mg Pd—C (10% Engelhardt 4505). MS: [M+1]⁺=219. ¹H-NMR (400 MHz, DMSO-d₆): 8.41 (q, 1H, NH), 7.24 (s, 1H), 7.19 (s, 1H), 6.98 (s, 1H), 3.41 (s, 2H, NH₂), 2.78 (d, 3H).

### Example 41

### 3-{3-[4-(6-Methylamino-pyrimidin-4-yloxy)-phenyl]-ureido}-N-methyl-5-trifluoromethyl-benzamide

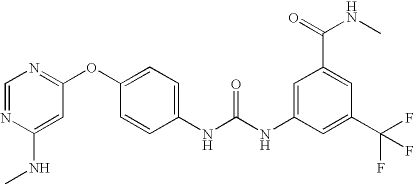

Prepared in analogy to Ex. 16 from 83 mg (0.18 mMol) 3-{3-[4-(6-chloro-pyrimidin-4-yloxy)-phenyl]-ureido}-N-methyl-5-trifluoromethyl-benzamide and 1.5 ml methylamine (33% in EtOH). MS: [M+1]⁺=461. ¹H-NMR (400 MHz, DMSO-d₆): 9.19 (s, 1H, NH), 8.87 (s, 1H, NH), 8.65 (q, 1H, NH), 8.13 (s, 1H), 8.03 (s, 1H), 7.75 (s, 1H), 7.5 (d, 2H), 7.26 (s, 1H), 7.07 (d, 2H) 5.72 (s, 1H), 3.59 (s, 3H), 2.82 (d, 3H).

### Example 42

### 3-{3-[4-(6-Azido-pyrimidin-4-yloxy)-phenyl]-ureido}-methyl-5-trifluoromethyl-benzamide

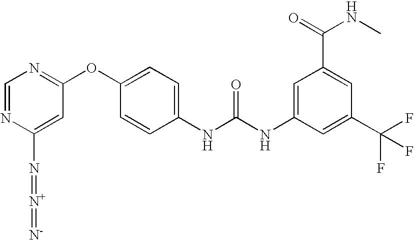

The title compound is prepared from 300 mg (0.64 mMol) of 3-{3-[4-(6-chloro-pyrimidin-4-yloxy)-phenyl]-ureido}-N-methyl-5-trifluoromethyl-benzamide as described in Ex. 7 to yield the title compound which is directly used as starting material in Ex. 43. MS: [M+1]⁺=473.

### Example 43

### 3-{3-[4-(6-Amino-pyrimidin-4-yloxy)-phenyl]-ureido}-N-methyl-5-trifluoromethyl-benzamide

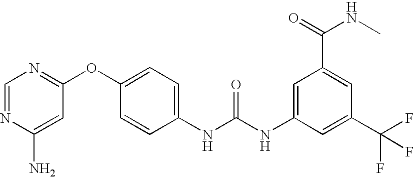

Hydrogenation of 0.3 g (0.64 mMol) of 3-{3-[4-(6-azido-pyrimidin-4-yloxy)-phenyl]-ureido}-N-methyl-5-trifluoromethyl-benzamide in 10 ml DME in the presence of 60 mg Pd/C 10% (“Engelhard 4505”), filtration and concentration of the filtrate gives the title compound: MS: [M+1]⁺=447; ¹H-NMR (DMSO-d₆): 9.17 (s, 1H, NH), 8.82 (s, 1H, NH), 8.60 (q, 1H, NH), 8.12 (s, 1H), 8.03 (s, 1H), 8.01 (s, 1H), 7.73 (s, 1H), 7.50 (d, 2H), 7.05 (d, 2H), 6.80 (s, 1H), 5.68 (s, 1H), 3.57 (s, 3H), 2.80 (d, 3H). HPLC ^{B}t_{Ret}: 1.82.

### Example 44

### N-[4-(6-Methylamino-pyrimidin-4-yloxy)-phenyl]-N′-(3-methylaminomethyl-5-trifluoromethyl-phenyl)-urea

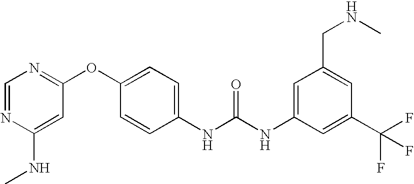

Can be synthesized analogously to the compounds described herein.

### Example 45

### N-[4-(2-Amino-pyrimidin-4-yloxy)-phenyl]-N′-[4-(4-isopropylpiperazin-1-ylmethyl)-3-trifluoromethyl-phenyl]-urea

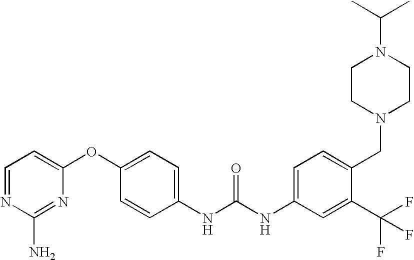

To a solution of 98 mg (0.33 mMol) triphosgene in 11 ml CH₂Cl₂ under N₂-atmosphere cooled in an ice bath, 302 mg (1.00 mMol) of 4-(4-isopropylpiperazin-1-ylmethyl)-3-trifluoromethyl-aniline (Step 15.2) and 0.14 ml (1.0 mMol) NEt₃ in 5 ml CH₂Cl₂ are added dropwise. After stirring for 10 min in the ice bath and 30 min at rt, a suspension of 202 mg (1.0 mMol) of 4-(4-amino-phenoxy)-pyrimidin-2-ylamine (Step 45.3) and 0.14 ml (1.0 mMol) NEt₃ in 5 ml CH₂Cl₂ is added during 5 min. After 15 min stirring at rt, the reaction mixture is concentrated in vacuo, the residue re-dissolved in CH₂Cl₂/MeOH and after addition of SiO₂ again concentrated. The resulting powder is put on top of a MPLC chromatography column and the title compound eluted with CH₂Cl₂/methanol (+1% NH₃^{aq}) 19:1→9:1 and finally lyophilized from dioxane: Anal. C₂H₃₀N₇F₃O₂.1.2H₂O.0.1 C₄HBO₂: C, H, N, F; MS: [M+1]⁺=530; ¹H-NMR (DMSO-d₆): 9.06 (s, HN), 8.86 (s, HN), 8.10 (d, 5.5 Hz, 1H), 7.98 (d, 2.3 Hz, 1H), 7.65 (d, 8.6 Hz, 1H), 7.59 (dd, 8.6 Hz, 2.3 Hz, 1H), 7.50 (d, 9.0 Hz, 2H), 7.10 (d, 9.0 Hz, 2H), 6.62 (s, H₂N), 6.09 (d, 5.5, 1H), 3.54 (s, 2H), 2.67 (m, 1H), 2.50 (m, 4H), 2.41 (m, 4H), 0.99 (d, 6.7 Hz, 6H).

The starting material is prepared as follows:

### Step 45.1: 2-Chloro-4-(4-nitro-phenoxy)-pyrimidine

18 g (130 mMol) 2,4-dichloropyrimidine dissolved in 100 ml of acetone are slowly added to a solution of 5.32 g (130 mMol) NaOH and 16.64 g (118.4 mMol) 4-nitrophenol in 100 ml H₂O at 0° C. After stirring for 23 h at 80° C., the reaction mixture is concentrated under reduced pressure, cooled, and the precipitated crude product is filtered off, washed with H₂O and dried in vacuo. Purification is performed by flash chromatography (SiO₂; 4.5×46 cm, hexane/EtOAc 2: 1): MS: [M+1]⁺=252; ¹H-NMR (400 MHz, DMSO-d₆): 8.67 (d, 4.5 Hz, 1H, pyrimidinyl), 8.33 (d, 8.5 Hz, 2H, phenyl), 7.56 (d, 8.5 Hz, 2H, phenyl), 7.31 (d, 4.5 Hz, 1H, pyrimidinyl), R_{f} (hexane/EtOAc=1:1): 0.38, HPLC ^{B}t_{Ret}: 5.97.

### Step 45.2: 4-(4-Nitro-phenoxy)-pyrimidin-2-ylamine

4 g (15.9 mMol) 2-chloro-4-(4-nitro-phenoxy)-pyrimidine dissolved in 100 ml EtOH and 100 ml aqueous NH₃ (25%) is stirred in an autoclave (2 bar) at 100° C. of 2 h. After concentrating the reaction mixture under reduced pressure, the precipitating product is taken up in MeOH and flash chromatographed (SiO₂; 4.5×26 cm, EtOAc/hexane/NH₃ 50:50:1.5→100:50:1.5) to give the title compound as white solid: R_{f} (EtOAc/hexane/NH₃: 100:50:1.5): 0.10; MS: [M+1]⁺=233.

### Step 45.3: 4-(4-Amino-phenoxy)-pyrimidin-2-ylamine

1.68 g (6.7 mMol) 4-(4-nitro-phenoxy)-pyrimidin-2-ylamine dissolved in 50 ml MeOH is hydrogenated in the presence of 500 mg Raney-Ni during 4 h. After filtering over Hyflo and washing twice with 40 ml EtOH, the reaction solution is concentrated under reduced pressure and flash chromatographed (SiO₂; 4.5×26 cm, EtOAc/hexane/NH₃ 100:50:1.5→200:50:1.5) to give the title compound as a beige solid: R_{f} (EtOAc/hexane/NH₃: 100:50:1.5): 0.10; MS: [M+1]⁺=203.

### Example 46

### N-[4-(2-Methylamino-pyrimidin-4-yloxy)-phenyl]-N′-[4-(4-isopropylpiperazin-1-ylmethyl)-3-trifluoromethyl-phenyl]-urea

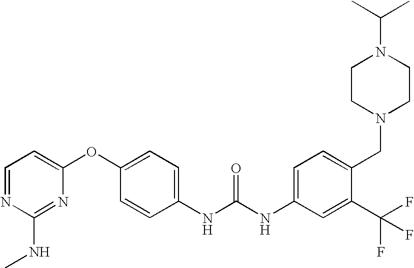

To a solution of 60 ma (0.20 mMol) triphosgene in 7 ml CH₂Cl₂ under N₂-atmosphere cooled in an ice bath, 181 mg (0.60 mMol) of 4-(4-isopropylpiperazin-1-ylmethyl)-3-trifluoromethyl-aniline (Step 15.2) and 83 pd (0.6 mMol) NEt₃ in 3-ml CH₂Cl₂ are added dropwise. After stirring for 10 min in the ice bath and 30 min at rt, a suspension of 130 mg (0.60 mMol) of [4-(4-amino-phenoxy)-pyrimidin-2-yl]-methyl-amine (Step 46.2) and 83 μl (0.6 mMol) NEt₃ in 3 ml CH₂Cl₂ is added during 5 min. After 90 min stirring at rt, the reaction mixture is concentrated in vacuo, the residue re-dissolved in CH₂Cl₂/MeOH and after addition of SiO₂ again concentrated. The resulting powder is put on top of a MPLC chromatography column and the title compound eluted with CH₂Cl₂/methanol (+1% NH₃^{aq}) 97:3→93:7: MS: [M+1]⁺=544; ¹H-NMR (CD₃OD/CDCl₃): 7.99 (d, 5 Hz, 1H), 7.67 (d, 2 Hz, ₁H), 7.60 (dd, 8.6 Hz, 2 Hz, 1H), 7.55 (d, 8.6 Hz, 1H), 7.43 (d, 9.0 Hz, 2H), 7.03 (d, 9.0 Hz, 2H), 5.96 (d, 5 Hz, 1H), 3.59 (s, 2H), 2.99 (m, 1H), 2.83 (s, H₃C), 2.70 (m, 4H), 2.58 (m, 4H), 1.12 (d, 6.3 Hz, 6H).

The starting material is prepared as follows:

### Step 46.1: Methyl-[4-(4-nitro-phenoxy)-pyrimidin-2-yl]-amine

2 g (7.95 mMol) 2-chloro-4-(4-nitro-phenoxy)-pyrimidine dissolved in 40 ml MeNH₂ (30% in EtOH) is stirred at rt for 50 min. After evaporation of the solvent, the crude product is flash chromatographed (SiO₂; 4.5×30 cm, hexane/EtOAc 1:1) to give the title compound as a white solid: R_{f} (hexane/EtOAc 2:1): 0.18; MS: [M+1]⁺=247; ¹H-NMR (400 MHz, CDCl₃): 8.33 (d, 8.5 Hz, 2H, phenyl), 8.24 (d/broad, 1H, pyrimidinyl), 7.35 (d, 8.5 Hz, 2H, phenyl), 6.22 (d, 6.0 Hz, 1H, pyrimidinyl), 2.90 (s/broad, 3H, CH₃).

### Step 46.2: [4-(4-Amino-phenoxy)-pyrimidin-2-yl]-methyl-amine

The title compound is prepared by hydrogenation in the presence of Raney-Ni from methyl-[4-(4-nitro-phenoxy)-pyrimidin-2-yl]-amine: R_{f} (hexane/EtOAc 1:1): 0.13; MS: [M+1]⁺=217; ¹H-NMR (400 MHz, DMSO-d₆): 8.04 (s/broad, 1H, pyrimidinyl), 6.95 (s/broad, 1H, HN), 6.76 (d, 8.5 Hz, 2H, phenyl), 6.54 (d, 8.5 Hz, 2H, phenyl), 5.90 (s/broad, 1H, pyrimidinyl), 5.00 (s, 2H, NH₂), 2.70 (s/broad, 3H, CH₃).

### Example 47

### N-[4-(2-Chloro-pyrimidin-4-yloxy)-phenyl]-N′-[4-(dimethylamino-methyl)-3-trifluoromethyl-phenyl]-urea

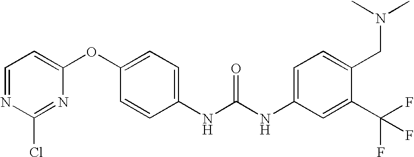

The title compound is prepared from 2-chloro-4-(4-isocyanato-phenoxy)-pyrimidine and 4-(dimethylamino-methyl)-3-trifluoromethyl-phenylamine.

The compounds of Ex. 48-50 can be prepared analogously to the procedures described herein:

### Example 48

### N-[4-(2-Methylamino-pyrimidin-4-yloxy)-phenyl]-N′-[4-(4-dimethylamino-methyl) 3-trifluoromethyl-phenyl]-urea

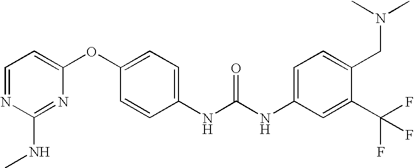

Prepared according to Ex. 45 from 101 mg (0.43 mMol) of 4-(N,N-dimethylamino methyl)-3-trifluoromethyl-aniline (Step 20.1-2) and 100 mg (0.43 mMol) of [4-(4-amino-phenoxy)-pyrimidin-2-yl]-methyl-amine (Step 46.2). After 3 h stirring at rt, the reaction mixture is concentrated in vacuo, the residue re-dissolved in CH₂Cl₂/MeOH and the crude product is purified by preparative TLC(CH₂Cl₂/MeOH 9:1) to give the title compound MS: [M+1]⁺=461; HPLC ^{B}t_{Ret}: 2.03, R_{f} (CH₂Cl₂/MeOH 9:1): 0.65.

### Example 49

### N-[4-(2-Methylamino-pyrimidin-4-yloxy)-phenyl]-N′-[4-(4-tert-butylpiperazinyl-methyl)-3-trifluoromethyl-phenyl]-urea

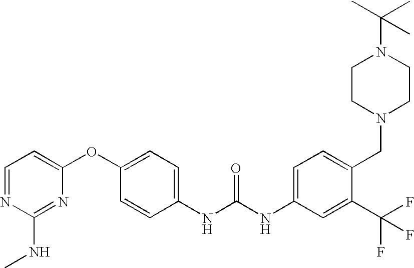

Prepared according to Ex. 45 from 146 mg (0.43 mMol) of 4-(4-^{tert}butyl-piperazin-1-ylmethyl)-3-trifluoromethyl-phenyl-amine (Step 20.5) and 100 mg (0.43 mMol) of [4-(4-aminophenoxy)-pyrimidin-2-yl]-methyl-amine (Step 46.2) and 83 μl (0.6 mMol) NEt₃ in 3 ml CH₂Cl₂ is added during 5 min. After 0.5 h stirring at rt, the precipitated product is isolated by filtration. MS: [M+1]⁺=558; m.p. 257-258° C., ¹H-NMR (400 MHz, DMSO-d₆): 9.60 (bs, 1H, NH), 9.09 (s, 1H, NH), 8.78 (s, 1H, NH), 8.10 (d, 1H,), 7.86 (s, 1H), 7.69-7.55 (m, 2H), 7.48 (d, 2H), 7.08 (d, 2H), 6.50 (bs, 1H, NH), 6.04 (d, 1H), 3.70 (s, 2H), 3.49-3.37 (m, 4H), 3.10-2.87 (m, 4H), 2.85 (s, 3H), 1.37 (s, 9H).

### Example 50

### N-[4-(2-Amino-pyrimidin-4-yloxy)-phenyl]-N′-[4-(4-tert-butylpiperazinyl-methyl 3-trifluoromethyl-phenyl]-urea

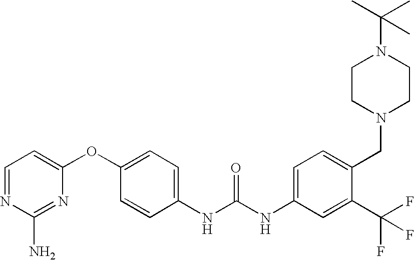

Prepared according to Ex. 45 from 312 mg (0.98 mMol) of 4-(4-^{tert}butyl-piperazin-1-ylmethyl)-3-trifluoromethyl-phenyl-amine (Step 20.5) 200 mg (0.98 mMol) of 4-(4-amino-phenoxy)-pyrimidin-2-ylamine (Step 45.3). After 30 min stirring at rt the precipitated product is isolated by filtration and washed with cold THF and dried in vacuo to give the title compound as a white solid. MS: [M+1]⁺=548; ¹H-NMR (DMSO-d₆): 9.41 (s, 1H, HN), 9.17 (s, 1H, NH), 8.03 (d, 1H), 7.97 (s, 1H), 7.62-7.58 (m, 2H), 7.43 (d, 2H), 7.02 (d, 2H), 6.59 (bs (2H), 6.01 (d, 1H), 3.62 (s, 2H), 3.49-3.39 (m, 2H), 2.99-2.82 (m, 4H), 2.61-2.48 (m, 2H), 1.17 (s, 9H).

The starting material (amine component) is prepared as described in Example 20, Steps 1-5.

### Example 51

### The Following Compounds can be Prepared Analogously

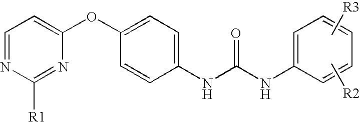

| | 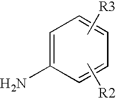 Eductfrom Step: | 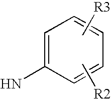 | R1 | HPLC^{A}t_{Ret}[min] | m.p.[° C.] | MS[M + 1]⁺ | Anal. |
|---|---|---|---|---|---|---|---|
| a.1)¹⁾ a.2)²⁾ | 23.2 23.2 | 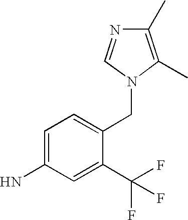 | HPLC^{A}t_{Ret}[min] | 8.8 9.2 | 215-216214-215 | 498 512 | CHNFH₂OCHNFH₂O |
| b.1)¹⁾b.2)²⁾ | 24.224.2 | 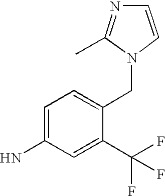 | NH₂NH—CH₃ | 8.5 8.9 | 216-217 | 484498 | CHNCHNF |
| c.1)¹⁾c.2)²⁾ | | 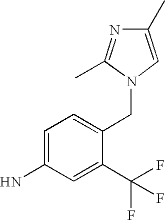 | NH₂NH—CH₃ | | | | |
| d.1)¹⁾d.2)²⁾ | 26.426.4 | 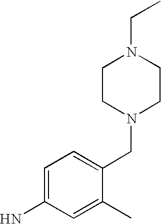 | NH₂NH—CH₃ | 6.3 6.8 | | 462476 | |
| e.1)¹⁾e.2)²⁾ | 27.127.1 | 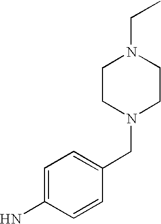 | NH₂NH—CH₃ | 6.7 | | 448462 | CHN |
| f.1)¹⁾f.2)²⁾ | 65.365.3 | 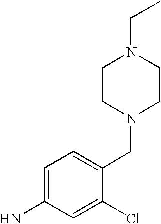 | NH₂NH—CH₃ | 7.1 7.7 | | 482496 | CHNCl |
| g.1)²⁾g.2)²⁾ | | 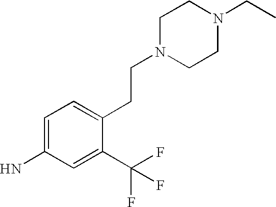 | NH₂NH—CH₃ | 6.7 | | 530544 | CHNF |
| h.1)¹⁾h.2)²⁾ | | 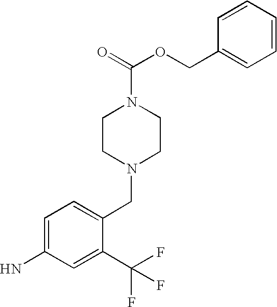 | NH₂NH—CH₃ | 10.2 | 105 | 622 | |

¹⁾ prepared analogously to Ex. 45;
²⁾ prepared analogously to Ex. 46

### Example 52

### Analogously to Example 45 the Following Compounds are Prepared

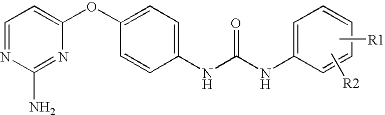

| | Source of 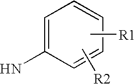 | 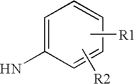 | HPLC^{B}t_{Ret} | R_{f} | MS[M + H] |
|---|---|---|---|---|---|
| a) | Step 14.4 | 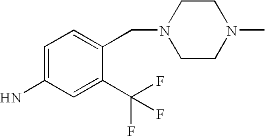 | 3.45 | (MeOH/CH₂Cl₂10:90): 0.36 | 502 |
| b) | commerciallyavailable | 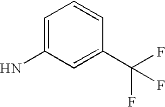 | [symbol] | (EtOAc/hexane4:1): 0.19 | [symbol] |
| c) | Step 52c.2 | 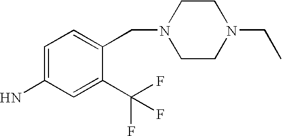 | 4.04 | (MeOH/CH₂Cl₂/NH₃ 12:87:1):0.37 | 516 |
| d) | Step 52d.1 | 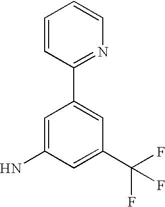 | 4.12 | (MeOH/CH₂Cl₂1:4): 0.32 | 467 |

[symbol]

indicates data missing or illegible when filed

### Step 52c.1: N-[4-(4-Ethyl-piperazin-1-ylmethyl)-3-trifluoromethyl-phenyl]-2,2,2-trifluoro-acetamide

2 g (5.71 mMol) of N-(4-bromomethyl-3-trifluoromethyl-phenyl)-2,2,2-trifluoro-acetamide (Step 14.2) and 2.22 ml (17.14 mMol) of N-ethylpiperazine, dissolved in 55 ml acetonitrile are stirred for 45 min at rt. After evaporating the acetonitrile under reduced pressure, the reaction mixture is diluted with 80 ml H₂O and extracted 3 times with 70 ml of EtOAc. The combined organic phases are washed twice with 30 ml NaHCO₃ solution and 30 ml brine, dried (MgSO₄), concentrated under reduced pressure and flash chromatographed (SiO₂; 4.0×24 cm, MeOH/CH₂Cl₂ 1:19) to give a yellow solid: R_{f}(MeOH/CH₂Cl₂ 1:4): 0.42; MS: [M+1]⁺=384; ¹H-NMR (400 MHz, DMSO-d₆): 11.40 (s/broad, 1H, NH), 8.02 (s, 1H), 7.90 (d, 7.5 Hz, 1H), 7.74 (d, 7.5 Hz, 1H), 3.56 (s, 2H, CH₂-aryl), 2.30 (m, 10H), 2.51 (t, 7.5 Hz, 3H, CH₃).

### Step 52c.2: 4-(4-Ethyl-piperazin-1-ylmethyl)-3-trifluoromethyl-phenylamine

A solution of 1.59 g (4.1 mMol) N-[4-(4-ethyl-piperazin-1-ylmethyl)-3-trifluoromethyl-phenyl]-2,2,2-trifluoro-acetamide in 41 ml MeOH and 20.5 ml of a 1M solution of K₂CO₃ in H₂O is stirred under Ar at 70° C. for 1.5 h. After evaporating the MeOH under reduced pressure, the reaction mixture is diluted with 80 ml H₂O and extracted 3× with 60 ml of EtOAc. The combined organic phases are washed with 30 ml H₂O and 30 ml brine, dried (MgSO₄), concentrated under reduced pressure and flash chromatographed (SiO₂; 4.0×24 cm, MeOH/CH₂Cl₂ 1:19) to give the title compound as yellow solid: R_{f} (MeOH/CH₂Cl₂ 1:4): 0.42; MS: [M+1]⁺=288; ¹H-NMR (400 MHz, DMSO-d₆): 7.24 (d, 7.5 Hz, 1H), 6.81 (s, 1H), 6.73 (d, 7.5 Hz, 1H) 5.41 (s, 2H, CH₂-aryl), 3.35 (m, 2H, CH—CH₃), 2.30 (m, 8H, piperazinyl), 2.51 (t, 6.5 Hz, 3H, CH₃).

### Step 52d.1: 3-Pyridin-2-yl-5-trifluoromethyl-phenylamine

The title compound is synthesized according to the procedure of [Lam F, Chan K S (1995), Synthesis of acyclic dinucleating Schiff base-pyridine and Schiff base-phosphine ligands. Tetrahedron Lett; 36(6):919-922] by stirring of 600 mg (2.44 mMol) of 3-amino-5-bromobenzotrifluoride, 1 g (2.69 mMol) 2-(tributylstannyl)-pyridine and 285 mg tetrakistriphenylphosphine Pd, dissolved in 10 ml THF for 7 d under Ar at 90° C. Chromatographic separation (SiO₂; 4.5×19 cm, EtOAc/hexane 1:2→2:3) gives the title compound as a slightly brownish solid: R_{f}(hexane/EtOAc 2:1): 0.17; MS: [M+1]⁺=239; ¹H-NMR (400 MHz, DMSO-d₆): 8.81 (d, 4.5 Hz, 1H, pyridinyl), 7.88 (m, 2H, pyridinyl), 7.53 (s, 1H, phenyl-CF₃), 7.43 (s, 1H, phenyl-CF₃), 7.37 (m, 1H, pyridinyl), 6.89 (s, 1H, phenyl-CF₃), 5.73 (s, 2H, NH₂).

### Example 53

### Analogously to Example 46 the Following Compounds are Prepared

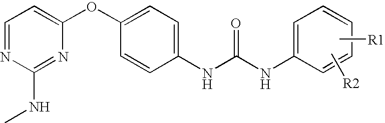

| | 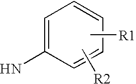 | HPLC^{B}t_{Ret} | R_{f} | MS[M + H] |
|---|---|---|---|---|
| a) | 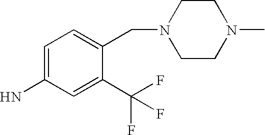 | 3.63 | (MeOH/CH₂Cl₂1:9): 0.15 | 516 |
| b)* | 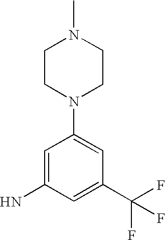 | 3.76 | (MeOH/CH₂Cl₂1:4): 0.15 | 502 |
| c) | 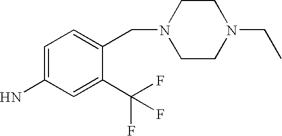 | 3.52 | (MeOH/CH₂Cl₂1:9): 0.10 | 530 |
| d)* | 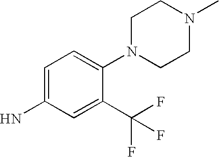 | 3.76 | (MeOH/CH₂Cl₂1:9): 0.15 | 502 |

*Synthesis of corresponding trifluoromethyl phenylamine building blocks is described under Step 53b.3 and 53d.1, respectively.

### Step 53b.1: (3-Bromo-5-trifluoromethyl-phenyl)-carbamic acid tert-butyl ester

A solution of 25 g (104 mMol) of 3-bromo-5-trifluoromethyl-aniline, 24 g (110 mMol) (Boc)₂O and 1.2 g (10 mMol) DMAP in 200 ml MeCN is stirred at 60° C. for 10 h. After evaporating the solvent under reduced pressure, the residue is flash chromatographed (SiO₂; hexane/EtOAc 10:1) and crystallized from hexane to give the title compound as white crystals: R_{f} (hexane/EtOAc 1:5): 0.23; MS: [M+1]⁺=341.

### Step 53b.2: [3-(4-Methyl-piperazin-1-yl)-5-trifluoromethyl-phenyl]-carbamic acid tert-butyl ester

6.8 g (20 mMol) (3-bromo-5-trifluoromethyl-phenyl)-carbamic acid tert-butyl ester, 2.6 ml (24 mMol) 1-methyl-piperazine, 2.7 g (28 mMol) NaOtBu, 6 ml tri-tert-butylphosphine (10% in hexane, 3 mMol) and 0.5 g (1 mMol) tris-(dibenzylidenaceton)-di-palladium dissolved in 100 ml toluene are stirred under Ar at 70° C. for 6 h. The reaction solution is diluted with 200 ml EtOAc and filtered over Hyflo. After washing with 50 ml of brine, the filtrate is dried (MgSO₄), concentrated under reduced pressure, and re-precipitated from EtOAc/hexane to give the title compound as a brownish oil: R_{f} (MeOH/CH₂Cl₂ 1:5): 0.45; MS: [M+1]⁺=360.

### Step 53b.3: 3-(4-Methyl-piperazin-1-yl)-5-trifluoromethyl-phenylamine

A solution of 3.2 g (8.9 mMol) of [3-(4-methyl-piperazin-1-yl)-5-trifluoromethyl-phenyl]-carbamic acid tert-butyl ester dissolved in 60 ml of 2.5 N HCl in 2-propanol is stirred at 60° C. for 5.5 h. After evaporating the solvent under reduced pressure, the residue is partitioned between 200 ml EtOAc and 100 ml NaHCO₃ solution. The organic phase is washed with 50 ml brine, dried (MgSO₄), and the solvent evaporated to give the title compound as brownish oil: MS: [M+1]⁺=260; R_{f} (MeOH/CH₂Cl₂ 1:5): 0.18; ¹H-NMR (400 MHz, DMSO-d₆): 6.31 (s, 1H), 6.27 (s, 1H), 5.34 (s, 1H), 3.32 (s/broad, 2H, NH₂), 3.70/2.42 (m/m, 4H/4H, CH₂-piperazinyl), 2.20 (s, 3H, CH₃).

### Step 53d.1: 4-(4-Methyl-piperazin-1-yl)-3-trifluoromethyl-phenylamine

The title compound is synthesized by nucleophilic substitution reaction from 1-bromo-4-nitro-2-trifluoromethyl-benzene with the 1-methyl-piperazine (140° C., 4 h) and further hydrogenolytic reduction of the nitro function to the amine by means of Raney nickel: m.p.: 121-123° C.; R_{f} (MeOH/CH₂Cl₂=1:5): 0.17; MS: [M+1]⁺=260; ¹H-NMR (400 MHz, DMSO-d₆): 7.21 (d, 9 Hz, 1H), 6.74 (m, 2H), 5.35 (s/broad, 2H, NH₂), 2.70 (m/broad, 4H, CH₂), 2.36 (s/broad, 4H, CH₂), 2.18 (s, 3H, CH₃).

### Example 54

### 1-[4-(6-Amino-pyrimidin-4-yloxy)-phenyl]-3-[3-(6-methyl-pyridin-2-yl)-5-trifluoromethyl-phenyl]-urea

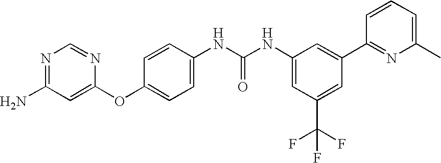

A solution of 252 mg (1 mMol) of 3-(6-methyl-pyridin-2-yl)-5-trifluoromethyl-phenylamine (Step 54.2) and 0.12 ml NEt₃ in 4.5 ml CH₂Cl₂ is added to 99 mg (0.33 mMol) triphosgene dissolved in 9 ml CH₂Cl₂ at 0° C. After stirring at rt for 15 min, a solution of 202 mg (1 mMol) 4-(4-amino-phenoxy)-pyrimidin-6-ylamine (Step 54.3) and 0.12 ml NEt₃ in 4.5 ml CH₂Cl₂ and 0.5 ml DMF is added. After stirring the brownish reaction solution at rt for 3.5 h, the solvent is evaporated under reduced pressure and flash chromatographed (SiO₂; 2.5×18 cm, MeOH/CH₂Cl₂/NH₃ 5:95:0.5) to give the title compound as beige solid: R_{f} (MeOH/CH₂Cl₂/NH₃ 5:95:0.5): 0.06; MS: [M+1]⁺=481; ¹H-NMR (DMSO-d₆): 9.21/8.83 (s/s, 1H/1H, urea), 8.29 (s, 1H, pyrimidinyl), 8.06 (m, 2H, pyridinyl), 7.93 (s, 1H, phenyl-CF₃), 7.80 (s, 1H, phenyl-CF₃), 7.79 (s, 1H, phenyl-CF₃), 7.51 (d, 9.0 Hz, 2H, phenyl), 7.26 (m, 1H, pyridinyl), 7.06 (d, 9.0 Hz, 2H, phenyl), 6.77 (s, 2H, NH₂), 5.66 (s, 1H, pyrimidinyl), 2.51 (s, 3H, CH₃).

### Step 54.1: 6-Methyl-2-(tributylstannyl)-pyridine

The title compound is synthesized analogously to the procedure of Zhang et al. (Synthetic Communications 31 (2001), 1129). To a solution of 3.83 g (22.2 mMol) 2-bromo-6-methylpyridine in 7 ml THF, 13.9 ml ⁿBuLi (1.6 N in hexane; 22.2 mMol) are added slowly at −78° C. under Ar. After stirring at −78° C. for 1.5 h, 6 ml (22.2 mMol) tributylstannylchloride are slowly added and the reaction solution is stirred for additional 30 min at −78° C. After filtration of the reaction mixture, the title compound is isolated by flash chromatography (SiO₂; 5×16 cm, EtOAc/hexane 1:9): colorless oil: R_{f} (hexane/EtOAc 3:2): 0.42; MS: [M+1]⁺=380.

### Step 54.2: 3-(6-Methyl-pyridin-2-yl)-5-trifluoromethyl-phenylamine

1 g (4.19 mMol) 3-amino-5-bromobenzotrifluoride, 1 g (2.60 mMol) 6-methyl-2-(tributylstannyl)-pyridine and 30 mg tetrakistriphenylphosphine Pd dissolved in 1.5 ml THF are stirred in sealed tube in a microwave oven (Emrys Optimizer, personal chemistry, Sweden) under Ar at 140° C. for 1000 seconds. Chromatographic separation (SiO₂; 5×18 cm, EtOAc/hexane 1:9→2:3) gives the title compound as colorless oil: R_{f} (hexane/EtOAc 3:2): 0.42; MS: [M+1]⁺253; ¹H-NMR (400 MHz, DMSO-d₆): 7.62 (t, 6.5 Hz, 1H, pyridinyl), 7.74/7.70 (s/s, 1H/1H, phenyl-CF₃), 7.69 (d, 6.5 Hz, 1H, pyridinyl), 7.12 (d, 6.5 Hz, 1H, pyridinyl), 6.91 (s, 1H, phenyl-CF₃), 3.95 (s/broad, 2H, NH₂), 2.63 (s, 3H, CH₃).

### Step 54.3: 4-(4-Amino-phenoxy)-pyrimidin-6-ylamine

2.0 g (9.725 mMol) 4-(6-chloro-pyrimidin-4-yl-oxy)-aniline (Step 1.2) dissolved in 80 ml aq NH₃ (25%) and 60 ml EtOH are stirred in a sealed tube at 80° C. for 23 h. After evaporating the solvent under reduced pressure on a water bath at 40° C., the residue is flash chromatographed (SiO₂, 5.5×65 cm; CH₂Cl₂/MeOH 9:1) to give the title compound as white solid: R_{f} (CH₂Cl₂/MeOH=9:1): 0.37; MS: [M+1]⁺=203; ¹H-NMR (400 MHz, DMSO-d₆): 8.01 (s, 1H, pyrimidinyl), 6.74 (d, 9 Hz, 2H, phenyl), 6.70 (s, 2H, NH₂), 6.57 (d, 9 Hz, 2H, phenyl), 5.51 (s, 1H, pyrimidinyl), 5.03 (s, 2H, NH₂).

### Example 55

### Additional Compounds are Synthesized Via Urea Formation Anlogously to the Preparation of Compound of Example 54

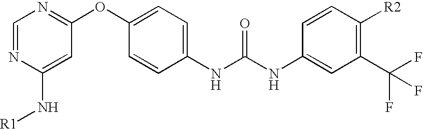

| | 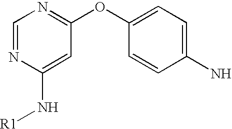 R1 = / source of theamine | 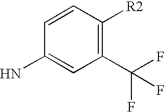 R2 = / sourceof the amine | HPLC^{B}t_{Ret} | R_{f} | MS[M + 1]⁺ |
|---|---|---|---|---|---|
| a) | 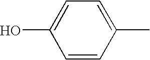 [symbol] | 4-Methyl-piperazin-1-ylmethyl/ Step 14.4 | | (NH₃/MeOH/CH₂Cl₂0.5:10:90): 0.38 | 608 |
| b) | 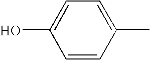 / Step 55a.1c* | 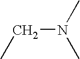 / Step 55b.2 | | (NH₃/MeOH/CH₂Cl₂1:10:90): 0.30 | 539 |
| c) | 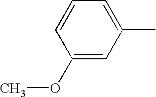 / Step 55c.1b | 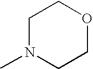 / Step 55c.2 | 6.64 | (MeOH/CH₂Cl₂5:95): 0.24 | 581 |
| d) | 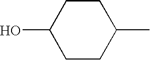 / Step 55d.1b | 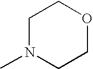 / Step 55c.2 | | (NH₃/MeOH/CH₂Cl₂1:10:90): 0.35 | 573 |

*The OH-group of the phenolic amine is TBDMS-protected. After urea formation, the TBDMS protecting group of the phenolic oxygen is split off by means of HF in pyridine (30%). [symbol]
indicates data missing or illegible when filed

### Step 55a.1a: 4-[6-(4-Nitro-phenoxy)-pyrimidin-4-ylamino]-phenol

3 g (11.9 mMol) 4-chloro-6-(4-nitro-phenoxy)-pyrimidine (Step 1.1), 1.95 g (17.9 mMol) 4-aminophenol, and 3.04 ml (17.9 mMol) of DIPEA dissolved in 50 ml 2-propanol are stirred at 85° C. for 18 h. After concentrating the reaction mixture under reduced pressure, the product precipitates as a colorless fine solid: R_{f} (EtOAc/hexane 2:1): 0.48; MS: [M+1]⁺=245; ¹H-NMR (400 MHz, DMSO-d₆): 9.40/9.25 (s/s, 2H, NH/OH), 8.28 (d, 7.5 Hz, 2H, phenyl-NO₂), 8.26 (s, 1H, pyrimidinyl), 7.40 (d, 7.5 Hz, 2H, phenyl-NO₂), 7.24 (d, 8.0 Hz, 2H, phenyl-OH), 6.77 (d, 8.0 Hz, 2H, phenyl-OH), 6.15 (s, 1H, pyrimidinyl).

### Step 55a.1b: [4-(tert-Butyl-dimethyl-silyloxy)-phenyl]-[6-(4-nitro-phenoxy)-pyrimidin-4-yl]-amine

1.5 g (4.63 mMol) of 4-[6-(4-nitro-phenoxy)-pyrimidin-4-ylamino]-phenol, 1.39 g (9.26 mMol) tert-butyl-dimethylsilyl chloride, 1.29 ml (9.26 mMol) NEt₃, dissolved in 20 ml DMF are stirred for 3.5 h. After concentrating the reaction mixture under reduced pressure and dissolving in phosphate buffer (50 ml, pH=7), the product is extracted by 10 ml EtOAc and purified by flash chromatography (SiO₂; 3.0×17 cm, EtOAc/hexane 1:1→4:1) to give the title compound as a colorless solid: MS: [M+1]⁺=439; ¹H-NMR (400 MHz, DMSO-d₆): 9.56 (s, 1H, NH), 8.28 (m, 3H, pyrimidinyl, phenyl-NO₂), 7.40 (m, 4H, phenyl-OTBS, phenyl-NO₂), 6.81 (d, 8.8 Hz, 2H, phenyl-OTBS, 6.20 (s, 1H, pyrimidinyl), 0.93 (s, 9H, TBS), 0.18 (s, 6H, TBS).

### Step 55a.1c: [6-(4-Amino-phenoxy)-pyrimidin-4-yl]-[4-(tertbutyldimethylsilanyloxy)-phenyl]-amine

1.8 g (4.1 mMol) of [4-(tert-butyl-dimethyl-silyloxy)-phenyl]-[6-(4-nitro-phenoxy)-pyrimidin-4-yl]-amine is hydrogenated in the presence of 0.4 g Raney-Ni in 50 ml EtOH/THF (35/15) during 3 h and purified by flash chromatography (SiO₂; 3.0×18 cm, EtOAc/hexane 1:1→4:1) to give the title compound as a cordless solid: R_{f} (EtOAc/hexane=2:1): 0.22; MS: [M+1]⁺=409; ¹H-NMR (400 MHz, DMSO-d₆): 9.22 (s, 1H, NH), 8.20 (s, 1H, pyrimidinyl), 7.37 (d, 8.8 Hz, 2H, phenyl-OTBS), 6.77 (d, 8.8 Hz, 2H, phenyl-NH₂), 6.70 (d, 8.8 Hz, 2H, phenyl-OTBS), 6.55 (8.8 Hz, 2H, phenyl-NH₂), 5.79 (s, 1H, pyrimidinyl), 5.02 (s, 2H, NH₂), 0.90 (s, 9H, TBS), 0.12 (s, 6H, TBS).

### Step 55b.2: 4-Dimethylaminomethyl-3-trifluoromethyl-phenylamine

1.8 g (5.14 mMol) of N-(4-bromomethyl-3-trifluoromethyl-phenyl)-2,2,2-trifluoro-acetamide (Step 14.2) dissolved in 25 ml HNMe₂ (30% in EtOH) is stirred at rt for 1 h and then (for saponification of the 2,2,2-trifluoro acetamide function) additionally at 50° C. for 3 h. After evaporating the solvent under reduced pressure, the residue is purified by flash chromatography (SiO₂; 5.5×17 cm, acetone/CH₂Cl₂/NH₃ 5:94:1→50:49:1) to give a yellowish oil: R_{f} (acetone/CH₂Cl₂/NH₃ 50:49:1): 0.73; MS: [M+1]⁺=219; ¹H-NMR (400 MHz, DMSO-d₆): 7.32 (d, 8.5 Hz, 1H), 6.88 (d, 4.5 Hz, 1H), 6.76 (d, 8.5 Hz, 1H), 5.44 (s, 2H, CH₂), 3.33 (s, 2H, NH₂), 2.12 (s, 6H, CH₃).

### Step 55c. 1a: (3-Methoxy-phenyl)-[6-(4-nitro-phenoxy)-pyrimidin-4-yl]-amine

5 g (19.9 mMol) of 4-chloro-6-(4-nitro-phenoxy)-pyrimidine (Step 1.1) and 4.88 ml (43.8 mMol) m-anisidine dissolved in 7.4 ml DIPEA and 85 ml 2-propanol are refluxed for 162 h. During concentrating the reaction mixture under reduced pressure, the residue precipitates to give the title compound as white crystals, which are washed with cold MeOH: MS: [M+1]⁺=339; ¹H-NMR (400 MHz, DMSO-d₆): 9.69 (s, 1H, NH), 8.40 (s, 1H, pyrimidinyl), 8.31 (d, 9.5 Hz, 2H, phenyl), 7.44 (d, 9.5 Hz, 2H, phenyl), 7.29 (s/broad, 1H, MeO-phenyl), 7.23 (t, 8.5 Hz, 1H, MeO-phenyl), 7.16 (d, 8.5 Hz, 1H, MeO-phenyl, 6.62 (d/broad, 8.5 Hz, 1H, MeO-phenyl), 7.97 (s/broad, 1H, pyrimidinyl), 5.11 (s, 2H, NH₂), 3.74 (s, 3H, CH₃).

### Step 55c.1b: [6-(4-Amino-phenoxy)-pyrimidin-4-yl]-(3-methoxy-phenyl)-amine

5.4 g (16 mMol) (3-methoxy-phenyl)-[6-(4-nitro-phenoxy)-pyrimidin-4-yl]-amine dissolved in 160 ml-MeOH/THF 2:1 is hydrogenated in the presence of Raney-Ni during 16 h. After filtering the reaction suspension over Hyflo and concentrating the reaction mixture, the title compound is precipitating as white crystals: MS: [M+1]⁺=309; ¹H-NMR (400 MHz, DMSO-d₀): 9.47 (s, 1H, NH), 8.36 (s, 1H, pyrimidinyl), 7.31 (s/broad, 1H, MeO-phenyl), 7.19 (t, 8.5 Hz, 1H, MeO-phenyl), 7.14 (d, 8.5 Hz, 1H, MeO-phenyl), 6.88 (d, 9.5 Hz, 2H, phenyl), 6.63 (d, 9.5 Hz, 2H, phenyl), 6.58 (d/broad, 8.5 Hz, 1H, MeO-phenyl), 7.97 (s/broad, 1H, pyrimidinyl), 5.06 (s/broad, 2H, NH₂), 5.11 (s, 2H, NH₂), 3.73 (s, 3H, CH₃); HPLC ^{B}t_{Ret}: 3.82.

### Step 55c.2: 4-Morpholin-4-yl-3-trifluoromethyl-phenylamine

The title compound is synthesized by nucleophilic substitution reaction from 1-bromo-4-nitro-2-trifluoromethyl-benzene with the morpholine (140° C., 4 h) and further hydrogenolytic reduction of the nitro function to the amine by means of Raney nickel: m.p.: 149-151° C.; R_{f} (hexane/EtOAc 1:1): 0.30; MS: [M+1]⁺=247.1, ¹H-NMR (400 MHz, DMSO-d₆): 7.22 (d, 9 Hz, 1H), 6.77 (m, 2H), 5.37 (s/broad, 2H, NH₂), 3.62 (m/broad, 4H, CH₂), 2.67 (m, broad 4H, CH₂).

### Step 55d.1a: 4-[6-(4-Nitro-phenoxy)-pyrimidin-4-ylamino]-cyclohexanol

300 mg (1.19 mMol) 4-chloro-6-(4-nitro-phenoxy)-pyrimidine (Step 1.1) and 184 mg (1.60 mMol) 4-amino-cyclohexanol, dissolved in 0.5 ml DIPEA and 30 ml 2-propanol are refluxed for 3 h. After evaporating the solvent, the residue is flash chromatographed twice (SiO₂; 2.5×12 cm, hexane/EtOAc 1:1→MeOH/EtOAc 5:95. SiO₂; 2×15 cm, 5→10% MeOH in CH₂Cl₂) to give a colorless oil: R_{f} (MeOH/CH₂Cl₂ 1:9): 0.50; MS: [M+1]⁺=331, ¹H-NMR (400 MHz, DMSO-d₆): 8.30 (d, 10.5 Hz, 2H, phenyl), 8.14 (s/broad, 1H, pyrimidinyl), 7.43 (d, 8.5 Hz, 1H, NH), 7.38 (d, 10.5 Hz, 2H, phenyl), 5.95 (s/broad, 1H, pyrimidinyl), 5.06 (s/broad, 2H, NH₂), 4.55 (d, 4.5 Hz, 1H, OH), 3.76 (s/broad, 1H, CH), 3.41 (m/broad, 1H, CH), 1.92-1.80 (m, 4H, CH₂), 1.25 (m, 4H, CH₂).

### Step 55d. 1b: 4-[6-(4-Amino-phenoxy)-pyrimidin-4-ylamino]-cyclohexanol

100 mg (0.30 mMol) of 4-[6-(4-nitro-phenoxy)-pyrimidin-4-ylamino]-cyclohexanol, dissolved in 15 ml MeOH are hydrogenated in the presence of Raney-Ni during 3 h. After filtering the reaction suspension over Hyflo and evaporating the solvent, the crude product is purified by flash chromatography (SiO₂; 2×20 cm, acetone/CH₂Cl₂/NH₃ 5:94:1→50:49:1) to give the title compound as a yellowish oil: R_{f} (MeOH/CH₂Cl₂/NEt₃ 15:84:1): 0.12; MS: [M+1]⁺=301; ¹H-NMR (400 MHz, DMSO-d₆): 8.09 (s, 1H, pyrimidinyl), 7.13 (d, 8.5 Hz, 1H, NH), 6.76 (d, 9.5 Hz, 2H, phenyl), 6.56 (d, 9.5 Hz, 2H, phenyl), 5.55 (s/broad, 1H, pyrimidinyl), 5.06 (s/broad, 2H, NH₂), 4.56 (d, 4.0 Hz, 1H, OH), [symbol] (s/broad, 1H, CH), 3.38 (m/broad, 1H, CH), 1.79 (m, 4H, CH₂), 1.23 (m, 4H, CH₂).

### Example 56

The following compounds can be prepared analogously:

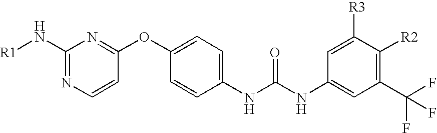

| | R1 | R2 | R3 |
|---|---|---|---|
| a) | H | 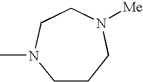 | H |
| b) | Me | 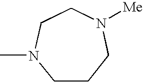 | H |
| c) | H | 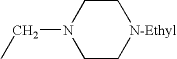 | CF₃ |
| d) | Me | 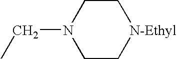 | CF₃ |
| e) | H | 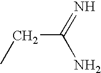 | CF₃ |
| f) | Me | 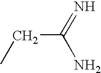 | CF₃ |
| g) | H | 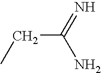 | H |
| h) | Me | 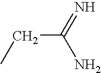 | H |

### Example 57

### 1-[4-(6-Amino-pyrimidin-4-yloxy)-phenyl]-3-(4-pyridin-2-yl-3-trifluoromethyl-phenyl)-urea

In a sealed tube, 150 mg (0.320 mMol) of 1-[4-(6-amino-pyrimidinyloxy)-phenyl]-3-(4-bromo-3-trifluoromethyl-phenyl)-urea (Step 57.3), 590 mg (1.602 mMol) of 2-(tributylstannyl)-pyridine and 97 mg (0.084 mMol) of tetrakis(triphenylphosphin)-palladium are suspended in 1,4-dioxane under an Argon atmosphere. After stirring for 2.5 h at 150° C. the solvent is removed under reduced pressure. Column chromatography (SiO₂; CH₂Cl₂/MeOH 95:5) and crystallization from ether gives the title compound as a white powder m.p.: 188-192° C.; R_{f} (CH₂Cl₂/MeOH 9:1): 0.19; MS: [M+1]⁺=470; HPLC ^{C}t_{Ret}=5.49.

The starting material is prepared as follows:

### Step 57.1: 1-(4-Bromo-3-trifluoromethyl-phenyl)-3-[4-(6-chloro-pyrimidin-4-yloxy)-phenyl]-urea

To a solution of 4.0 g (16.15 mMol) 4-chloro-6-(4-isocyanato-phenoxy)-pyrimidine (Example 1; Step 1.3) in 13 ml of THF under N₂-Atmosphere, 3.88 g (16.15 mMol) of 4-bromo-3-trifluoromethyl-aniline dissolved in 85 ml of ether is added. After stirring for 27 h at rt, the product is filtered off and washed with ether. After drying, the title compound is obtained as white crystals: m.p.: 179-182° C.; R_{f} (EtOAc):0.55; MS: [M+1]⁺=489; HPLC ^{C}t_{Ret}=7.46.

### Step 57.2: 1-[4-(6-Azido-pyrimidin-4-yloxy)-phenyl]-3-(4-bromo-3-trifluoromethyl-Phenyl)-urea

A mixture of 4.13 g (8.47 mMol) of 1-(4-bromo-3-trifluoromethyl-phenyl)-3-[4-(6-chloropyrimidin-4-yloxy)-phenyl]-urea and 1.1 g (16.94 mMol) of NaN₃ in 65 ml of DMF is stirred for 19 h at 50° C. and 6 h at 60° C. The reaction mixture is poured into 150 mL of water and extracted with EtOAc (3×350 mL). The organic layers are washed with water and brine, dried (Na₂SO₄) and concentrated. The crude product is directly used in the following hydrogenation step (Step 57.3). R_{f} (EtOAc): 0.58; MS: [M+1]⁺=494; HPLC ^{C}t_{Ret}=7.58.

### Step 57.3: 1-[4-(6-Amino-pyrimidin-4-yloxy)-phenyl]-3-(4-bromo-3-trifluoromethyl-phenyl)-urea

4.1 g (8.3 mMol) of 1-[4-(6-azido-pyrimidin-4-yloxy)-phenyl]-3-(4-bromo-3-trifluoromethyl-phenyl)-urea dissolved in 80 mL of EtOH is hydrogenated in the presence of 1 g Raney-Ni at rt during 15 h. The reaction solution is filtered and concentrated. Column chromatography (SiO₂; EtOAc) and crystallization from ether gives the title compound: m.p.: 186-188° C.; R_{f} (EtOAc):0.18; MS: [M+1]⁺=469; HPLC ^{C}t_{Ret}=5.49.

### Example 58

### 1-[4-(6-Amino-pyrimidin-4-yloxy)-phenyl]-3-(4-pyridin-3-yl-3-trifluoromethyl-phenyl)-urea

The title compound is prepared as described in Example 57 using 3-(1,1,1-tributylstannyl)pyridine: m.p.: 132-135° C.; MS: [M+1]⁺=467; HPLC ^{C}t_{Ret}=3.54.

### Example 59

### 1-[4-(6-Amino-pyrimidin-4-yloxy)-phenyl]-3-(4-pyridine-4-yl-3-trifluoromethyl-phenyl)-urea

The title compound is prepared as described in Example 57 using 4-(1,1,1-tributylstannyl)pyridine: m.p.: 131-135° C.; MS: [M+1]⁺=467; HPLC ^{C}t_{Ret}=3.51.

### Example 60

### 1-[4-(6-Amino-pyrimidin-4-yloxy)-phenyl]-3-[4-(6-methyl-pyridin-2-yl)-3-trifluoromethyl-phenyl]-urea

The title compound is prepared as described in Example 57 using 2-methyl-6-tributylstannyl-pyridine (Step 54.1): m.p.: 130-133° C.; MS: [M+1]⁺=481 HPLC ^{C}t_{Ret}=3.66.

### Example 61

### 1-[4-(6-Methylamino-pyrimidin-4-yloxy)-phenyl]-3-(4-pyridin-2-yl-3-trifluoromethyl-phenyl)-urea

In a sealed tube, 136 mg (0.282 mMol) of 1-(4-bromo-3-trifluoromethyl-phenyl)-3-[4-(6-methylamino-pyrimidin-4-yloxy)-phenyl]-urea (Step 61.1), 129 mg (0.35 mMol) of 2-(tributylstannyl)-pyridine and 36 mg (0.031 mMol) of tetrakis(triphenylphosphin)-palladium are suspended in 0.5 mL of THF under an Argon atmosphere. The reaction mixture is heated in a microwave oven (Emrys Optimizer) for 85 min at 140° C. After filtration, the mother liquor is evaporated and chromatographed (SiO₂; CH₂Cl₂MeOH 95:5). By means of preparative TLC (SiO₂; CH₂Cl₂/MeOH 9:1), the title compound is obtained as a white powder m.p.: 114-118° C.; R_{f} (CH₂Cl₂/MeOH 9:1): 0.32; MS: [M+1]⁺=481; HPLC ^{C}t_{Ret}=3.78.

The starting material is prepared as follows:

### Step 61.1: 1-(4-Bromo-3-trifluoromethyl-phenyl)-3-[4-(6-methylamino-pyrimidin-4-yloxy)-phenyl]-urea

3 g (6.15 mMol) of 1-(4-bromo-3-trifluoromethyl-phenyl)-3-[4-(6-chloro-pyrimidin-4-yloxy)-phenyl]-urea (Step 57.1) is dissolved in 35.5 mL of a 33% solution of MeNH₂ in EtOH and stirred in an ice bath for 4 h. After removal of the solvent under reduced pressure, the residue is chromatographed (SiO₂; EtOAc) and crystallized from ether to give the title compound as white crystals: m.p.: 161-164° C.; R_{f} (EtOAc): 0.26; MS: [M+1]⁺=482; HPLC ^{C}t_{Ret}=5.64.

### Example 62

### 1-[4-(6-Methylamino-pyrimidin-4-yloxy)-phenyl]-3-(4-pyridin-3-yl-3-trifluoromethyl-phenyl)-urea

The title compound is prepared as described in Example 61 using 3-(1,1,1-tributylstannyl)pyridine: m.p.: 118-123° C.; MS: [M+1]⁺=481; HPLC ^{C}t_{Ret}=3.67.

### Example 63

### 1-[4-(6-Methylamino-pyrimidin-4-yloxy)-phenyl]-3-(4-pyridin-4-yl-3-trifluoromethyl-phenyl)-urea

The title compound is prepared as described in Example 61 using 4-(1,1,1-tributylstannyl)pyridine: m.p.: 127-130° C.; MS: [M+1]⁺=481; HPLC ^{C}t_{Ret}=3.64.

### Example 64

### 1-[4-(6-Methylamino-pyrimidin-4-yloxy)-phenyl]-3-[4-(6-methyl-pyridin-2-yl)-3-trifluoromethyl-phenyl]-urea

The title compound is prepared as described in Example 61 using 2-methyl-6-tributylstannyl-pyridine (Step 54.1): m.p.: 106-109° C.; MS: [M+1]⁺=495; HPLC ^{C}t_{Ret}=3.80.

### Example 65

### N-[4-(6-Chloro-pyrimidin-4-yloxy)-phenyl]-N′-[4-(4-ethylpiperazin-1-ylmethyl)-3-chloro-phenyl]-urea

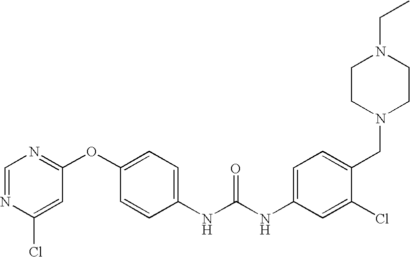

To a solution of 720 mg (2.8 mMol) of 4-(4-ethylpiperazin-1-ylmethyl)-6-chloro-aniline (Step 65.3) in 30 ml THF under N₂-atmosphere, 710 mg (2.86 mMol) 4-chloro-6-(4-isocyanatophenoxy)-pyrimidine (Step 1.3) are added. After stirring for 18 h, the reaction mixture is filtered, the filtrate partially concentrated and the title compound crystallized by addition of DIPE: MS: [M+1]⁺=501; ¹H-NMR (DMSO-d₆): 8.91 (s, 1H), 8.88 (s, 1H), 8.66 (s, 1H), 7.72 (d, 2 Hz, 1H), 7.54 (d, 9 Hz, 2H), 7.36 (d, 8 Hz, 1H), 7.35 (s, 1H), 7.28 (dd, 8 Hz, 2 Hz, 1H), 7.18 (d, 9 Hz, 2H), 3.49 (s, 2H), 2.43 (m, 8H), 2.32 (q, 7.1 Hz, 2H), 0.99 (t, 7.1 Hz, H₃C).

The starting material is prepared as follows:

### Step 65.1: (4-Nitro-2-chloro-phenyl)(4-ethylpiperazin-1-yl)-methanone

Analogously to Step 5.1, 5.0 g (24.8 mMol) of 4-nitro-2-chloro-benzoic acid are activated with 6.0 ml (71 mMol) of oxalylchloride and reacted with 6.6 ml (52 mMol) of 1-ethylpiperazine, yielding the title compound: MS: [M+1]⁺=298; HPLC ^{A}t_{Ref}=7.3.

### Step 65.2: (4-Amino-2-chloro-phenyl)-(4-ethylpiperazin-1-yl)-methanone

Hydrogenation of 7.29 g (24.5 mMol) of (4-nitro-2-chloro-phenyl)-(4-ethylpiperazin-1-yl)-methanone in 130 ml ethanol in the presence of 1.3 g of Raney-Nickel as described in Step 1.5 and crystallization from toluene gives the title compound: m.p.: 123-124° C.; MS: [M+1]⁺=268.

### Step 65.3: 4-(4-Ethylpiperazin-1-ylmethyl)-3-chloro-aniline

Analogously to Step 5.3, 5.06 g (18.9 mMol) (4-amino-2-chloro-phenyl)-(4-ethylpiperazin-1-yl)-methanone in 60 ml THF are reduced by 57 ml BH₃ (1M in THF). Chromatography (SiO₂; CH₂Cl₂/MeOH/NH₃^{aq} 95:5:1→80:20:1) gives the title compound: MS: [M+1]⁺=254; ¹H-NMR (CDCl₃): 7.21 (d, 8 Hz, 1H), 6.72 (d, 2.3 Hz, 1H), 6.58 (dd, 8 Hz, 2.3 Hz, 1H), 3.70 (s, H₂N), 3.57 (s, 2H), 2.6 (m, 8H), 2.47 (q, 7.2 Hz, 2H), 1.13 (t, 7.2 Hz, H₃C).

### Example 66

### 1-[4-(2-Amino-pyrimidin-4-yloxy-phenyl]-3-(4-piperazin-1-ylmethyl-3-trifluoromethyl-phenyl)-urea

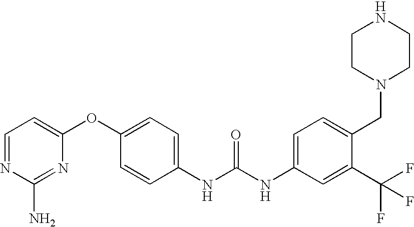

Hydrogenation of 107 mg (0.172 mMol) 4-(4-{3-[4-(2-amino-pyrimidin-4-yloxy)-phenyl]-ureido}-2-trifluoromethyl-benzyl)-piperazine-1-carboxylic acid benzyl ester (Ex. 51.h.1) in 6 ml methanol in presence of 20 mg Pd/C (10%; Engelhard 4505), filtration and Combi. Flash chromatography (CH₂Cl₂/MeOH+1% NH₃^{aq} 95:5→4:1) gives the title compound: R_{f} (CH₂Cl₂/MeOH/NH₃^{aq} 80:20:1): 0.10; HPLC ^{A}t_{Ret}=7.6; MS: [M+1]⁺=488; ¹H-NMR (CD₃OD): 8.09 (d, 5.9 Hz, 1H), 7.90 (m, 1H), 7.74 (d, 8.2 Hz, 1H), 7.64 (d, 8.2 Hz, 1H), 7.53 (d, 9.0 Hz, 2H), 7.12 (d, 9.0 Hz, 2H), 6.18 (d, 5.9 Hz, 1H), 3.63 (s, 2H), 2.88 (m, 4H), 2.48 (m, 4H).

### Example 67

### 1-[4-(2-Methylamino-pyrimidin-4-yloxy-phenyl]-3-(4-piperazin-1-ylmethyl-3-trifluoromethyl-phenyl)-urea

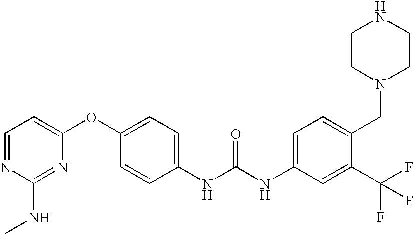

Can be prepared analogously to the procedures described herein.

### Example 68

### N-(6-{4-[3-(3-Trifluoromethyl-phenyl)-ureido]-phenoxy}-pyrimidin-1-yl)-acetamide

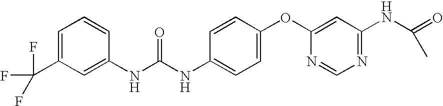

N-(4-(4-Chloropyrimidin-6-yl)-oxyphenyl)-N′-(3-trifluoromethyl-phenyl)-urea (Step 68.1) (100 mg, 0.245 mmol), acetamide (40 mg, 0.68 mmol), Pd₂(dba)₃ [tris(dibenzylideneacetone)dipalladium(0)] (6 mg), 4,5-bis(diphenylphosphino)-9,9-dimethylxanthrene (9 mg), and Cs₂CO₃ (160 mg) are stirred in THF (3 mL) at 55° C. for 8 h under Ar. After filtration and evaporation of the solvent, the product is isolated by preparative thin layer chromatography (4 20×20 cm plates, acetone/CH₂Cl₂=3:7): white solid, M+H=431.9, ¹H-NMR (400 MHz, DMSO-d₆): 10.85 (s, 1H, pyrimidinyl), 9.03/8.84 (s/s, 1H/1H, urea), 8.45 (s, 1H, NH), 7.98 (s, 1H, pyrimidinyl), 7.56 (d, 8.5 Hz, 1H), 7.56 (d/s, 9.0 Hz, 2H/1H), 7.29 (d, 8.5 Hz, 1H), 7.06 (d, 9.0 Hz, 2H), 2.09 (s, 3H, CH₃), R_{f} (acetone/CH₂Cl₂=3:7): 0.34.

### Step, 68.1 N-(4-(4-Chloropyrimidin-6-yl)-oxyphenyl)-N′-(3-trifluoromethyl-phenyl)-urea

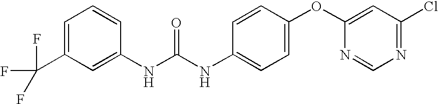

After stirring 3-trifluoromethyl-phenyl isocyanate (412 mg, 2.2 mmol), (4-(6-chloro-pyrimidin-4-yl-oxy)-aniline (Step 68.2; 0.25 g, 1.1 mmol); and pyridine (0.1-8 ml), dissolved in THF (3 ml) overnight, the reaction solution is concentrated under reduced pressure and flash chromatographed (silica gel, 2.5×17 cm; acetone/CH₂Cl₂=5:95→1:9) to give the title compound as a colorless solid: M+H=408.9/410.9, ¹H-NMR (400 MHz, DMSO-d₆): 9.07 (s, 1H, NH), 8.89 (s, 1H, NH), 8.63 (d, 2.0 Hz, 1H, pyridinyl), 8.01 (s, 1H, 3-CF₃-phenyl), 7.57 (d/broad, 8.0 Hz, 1H, CF₃-phenyl), 7.52 (d, 9.5 Hz, 2H, oxo-phenyl-amine), 7.50 (m, 1H, 3-CF₃-phenyl), 7.32 (d, 2.0 Hz, 1H, pyridinyl), 7.29 (d/broad, 8.0 Hz, 1H, —CF₃-phenyl, 7.15 (d, 9.5 Hz, 2H, oxo-phenyl-amine), (d, 6.5 Hz, 2H, pyridinyl); R_{f} (acetone/CH₂Cl₂=1:9): 0.54; m.p.=187.4-189.7° C.

The starting materials are prepared as follows:

### Step 68.2: (4-(6-chloro-pyrimidin-4-yl-oxy)-aniline

4-Chloro-6-(4-nitro-phenoxy)-pyrimidine (Step 68.3; 3.6 g, 14.3 mmol) dissolved in MeOH (250 ml) is hydrogenated in the presence of Raney-Ni (3 g) at 40° C. for 3 d. The reaction solution is filtered, concentrated under reduced pressure and crystallized from EtOAc/hexane to give 4-chloro-6-(4-amino-phenoxy)-pyrimidine: M+H=222/224; ¹H-NMR (400 MHz, DMSO-d₆): 8.62 (s, 1H, piperidinyl), 7.13 (s, 1H, piperidinyl), 6.83 (d, 9 Hz, 2H, phenyl), 6.56 (d, 9 Hz, 2H, phenyl), 5.12 (s, 2H, NH₂); m.p.=135.5-138.1° C.

### Step 68.3: 4-Chloro-6-(4-nitro-phenoxy)-pyrimidine

4-Nitrophenol (2.8 g, 20.1 mmol), 2,4-dichloro-pyrimidine (3 g, 20.1 mmol), NaOH (0.8 g, 20.1 mmol) dissolved in H₂O/acetone (80 ml; 1:1) are stirred at 60-65° C. for 1 h. The reaction solution is concentrated under reduced pressure and flash chromatographed (silica gel, 4.5×22 cm, EtOAc/hexane=1:4) to give the title compound as a colorless solid: M+H=252/254; ¹H-NMR (400 MHz, DMSO-d₆): 8.67 (s, 1H, pyrimidinyl), 8.34 (d, 9 Hz, 2H, phenyl), 7.58 (d, 9 Hz, 2H, phenyl), 7.53 (s, 1H, pyrimidinyl); R_{f} (EtOAc/hexane=1:1): 0.16; m.p.=125.4-126.6° C.

### Example 69

### N-(6-{4-[3-(4-Morpholin-4-yl-3-trifluoromethyl-phenyl)-ureido]-phenoxy}-pyrimidin-4-yl)-acetamide

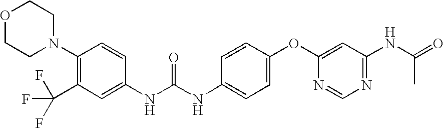

The title compound is prepared analogously to the synthesis of compound of Example 68 from 1-[4-(6-chloro-pyrimidin-4-yloxy)-phenyl]-3-(4-morpholin-4-yl-3-trifluoromethyl-phenyl)-urea (Step 69.1): beige solid, M+H=516.9, HPLC [20→100% CH₃CN (0.1% TFA) and H₂O (0.1% TFA) in 7 min and remaining at 100 CH₃CN (0.1% TFA) for 2 min]: t_{Ret}=7.72 min, R_{f} (MeOH/CH₂Cl₂=1:9): 0.42.

### Step 69.1:1-[4-(6-Chloro-pyrimidin-4-yloxy)-phenyl]-3-(4-morpholin-4-yl-3-trifluoromethyl-phenyl)-urea

The title compound is prepared analogously to the synthesis of compound of Example 1 starting from compound of Step 55c.2: white solid, M−H=491.9, HPLC [20→100% CH₃CN (0.1% TFA) and H₂O (0.1% TFA) in 7 min and remaining at 100 CH₃CN (0.1% TFA) for 2 min]: t_{Ret}=7.52 min, R_{f} (MeOH/CH₂Cl₂=3:97): 0.17.

### Example 70

### 6-(4-{3-[4-(4-Ethyl-piperazin-1-ylmethyl)-3-trifluoromethyl-phenyl]-ureido}-phenoxy)-pyrimidin-4-yl]-carbamic acid methyl ester

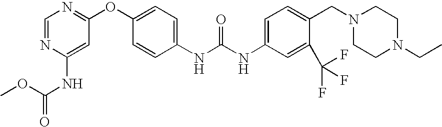

787 μl (10.2 mMol) methyl chloroformate dissolved in 10 ml CH₂Cl₂ are slowly added to a solution of 160 mg (0.31 mMol) 1-[4-(6-amino-pyrimidin-4-yloxy)-phenyl]-3-[4-(4-ethylpiperazin-1-ylmethyl)-3-trifluoromethyl-phenyl]-urea (Step 70.1), 5.6 ml pyridine and 20 mg DMAP in 16 ml CH₂Cl₂ at rt. After stirring for 2 h, the resulting suspension is filtered, the filtrate diluted with 100 ml EtOAc, washed twice with H₂O and brine. The aqueous layers are extracted twice with EtOAc, the organic phases dried (Na₂SO₄) and concentrated under reduced pressure. CombiFlash chromatography (CH₂Cl₂/NH₃^{aq}/MeOH 96:1:3→90:1:9) gives white crystals: m.p.: 191-193° C.; Anal. C₂₇H₃₀N₇F₃O₄: C, H, N; MS: [M+1]⁺=574.

### Step 70.1: 1-[4-(6-Amino-pyrimidin-4-yloxy)-phenyl]-3-]4-(4-ethyl-piperazin-1-ylmethyl)-3-trifluoromethyl-phenyl]-urea

The title compound is prepared analogously to the synthesis of compound of Ex. 19: Anal. C₂₅H₂₈N₇F₃O₂-0.86H₂O: C, H, N, F, H₂O; MS: [M+1]⁺=516; HPLC ^{A}t_{Ret}=8.0.

### Example 71

### 1-[4-(2-Acetylamino-pyrimidin-4-yloxy)-phenyl]-3-[4-(4-methyl-piperazin-1-ylmethyl)-3-trifluoromethyl-phenyl]-urea

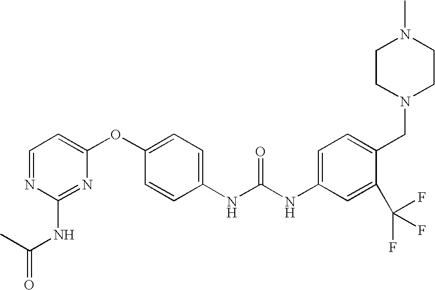

119 μl (1.67 mMol) acetylchloride dissolved in 7 ml CH₂Cl₂ are added during 2.5 h to a solution of 250 mg (0.50 mMol) 1-[4-(2-amino-pyrimidin-4-yloxy)-phenyl]-3-[4-(4-methylpiperazin-1-ylmethyl)-3-trifluoromethyl-phenyl]-urea (Ex. 52a) and 10 mg DMAP in 6.5 ml pyridine. After stirring for another hour, the mixture is diluted with 200 ml water and 250 ml EtOAc. The separated aqueous layer is re-extracted twice with EtOAc. The organic phases are washed with water and brine, dried (Na₂SO₄) and concentrated in vacuo. Reversed phase chromatography (Gilson System) gives the title compound: acetone/EtOH+1% Et₃N 95:5→4:1; MS: [M+1]₊=544; R_{f} (acetone/EtOH/Et₃N 80:20:1): 0.11; HPLC ^{A}t_{Ret}=7.8.

### Example 72

### The following compounds can be prepared analogously to the described procedures

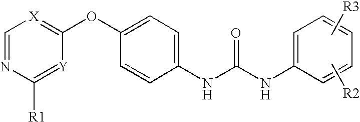

| | 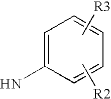 | X | Y | R1 | HPLC^{A}t_{Ret}[min] | m.p. [° C.] | MS[M + 1]⁺ | Anal. |
|---|---|---|---|---|---|---|---|---|
| a) | 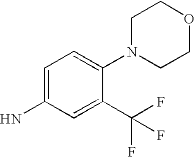 | N | CH | 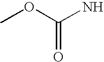 | 14.2 | | 533 | CHN |
| b) | 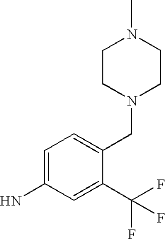 | CH | N | 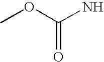 | 9.0 | | 560 | CHNF |
| c) | 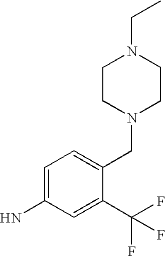 | CH | N | 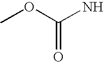 | 9.6 | | 574 | CHNF |

### Example 73

### 3-[3-(4-{6-[4-(tert-Butyl-dimethyl-silanyloxy)-phenylamino]-pyrimidin-4-yloxy}phenyl)-ureido]-5-trifluoromethyl-benzamide

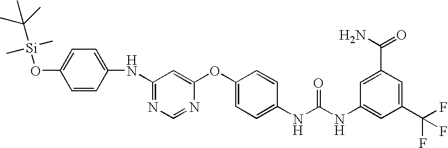

The title compound is prepared by urea formation from [6-(4-amino-phenoxy)-pyrimidin-4-yl]-[4-(tert-butyl-dimethyl-silanyloxy)-phenyl]-amine and 3-amino-5-trifluoromethyl-benzamide (Step 73.1) analogously to the preparation of compound of Ex. 54: MS: [M+1]⁺=639; R_{f} (MeOH/CH₂Cl₂=1:9): 0.49.

### Step 73.1 [6-(4-Amino-phenoxy)-pyrimidin-4-yl]-[4-(tert-butyl-dimethyl-silanyloxy)-phenyl]-amine

The title compound is prepared as described in WO 2003/099771.

### Example 74

### 1-(3′-Chloro-2-trifluoromethyl-biphenyl-4-yl)-3-{4-[2-(4-dimethylamino-butylamino)-pyrimidin-4-yloxy]-2-methyl-phenyl}-urea

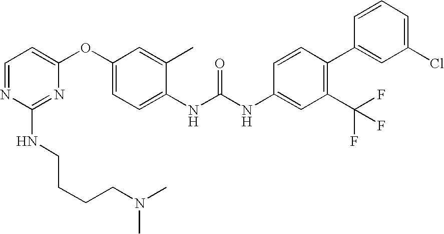

A solution of 3′-chloro-2-trifluoromethyl-biphenyl-4-amine (48 mg, 0.18 mMol) and DIPEA (67 μL, 0.38 mmol, 2.2 equiv) in CH₂Cl₂ (0.6 mL) is added dropwise to a cold (0° C.) solution of triphosgene (19 mg, 0.07 mMol) in CH₂Cl₂ (0.6 mL). Then, a solution of N-[4-(4-amino-3-methyl-phenoxy)-pyrimidin-2-yl]-N′,N′-dimethyl-butane-1,4-diamine (56 mg, 0.18 m Mol) and DIPEA (66 μL, 0.38 mMol, 2.2 equiv) in CH₂Cl₂ (1.1 mL) is added to the reaction mixture. The mixture is allowed to warm to rt, stirred for 10 min, and concentrated in vacuo. MPLC (CH₃CN/H₂O/TFA) purification of the crude material affords the title compound as a yellow solid: MS: 613.9 [M]⁺; HPLC ^{D}t_{Ret}=4.2.

### Step 74.1: N-[4-(4-Amino-3-methyl-phenoxy)-pyrimidin-2-yl]-N′,N′-dimethyl-butane-1,4-diamine

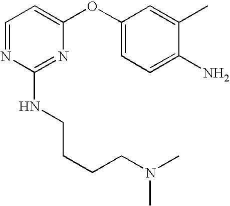

A mixture of [4-(2-chloro-pyrimidin-4-yloxy)-2-methyl-phenylamine (808 mg, 3.43 mMol), 4-dimethylaminobutylamine (438 mg, 3.77 mMol, 1.1 equiv), and K₂CO₃ (1.3 g, 9.26 mMol, 2.7 equiv) in DMF (8 mL) is stirred for 1 h at 100° C. The reaction mixture is allowed to cool to rt and filtered through a glass sintered funnel. The filtrate is concentrated in vacuo. Purification of the crude material by silica gel column chromatography (CH₂Cl₂/MeOH, 9:1→CH₂Cl₂/MeOH+1% NH₃^{aq}, 9:1) provides the title compound as a yellow oil: MS: 316.1 [M]⁺; R_{f}=0.23 (CH₂Cl₂/MeOH+1% NH₃^{aq} 4:1).

### Step 74.2: [4-(2-Chloro-pyrimidinyloxy)-2-methyl-phenylamine

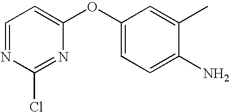

A mixture of 2-chloro-4-(3-methyl-4-nitro-phenoxy)-pyrimidine (992 mg, 3.73 mMol) and Raney-Ni (700 mg) in MeOH/THF (3:1, 40 mL) is stirred for 7 h at rt, under a hydrogen atmosphere. The reaction mixture is filtered through a pad of celite and the filtrate is concentrated in vacuo to afford the title compound as a yellow solid: MS: 236.0 [M+1]⁻; HPLC ^{D}t_{Ret}=2.2.

### Step 74.3: 2-Chloro-4-(3-methyl-4-nitro-phenoxy)-pyrimidine

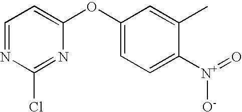

2,4-Dichloropyrimidine (3.7 g, 25.17 mMol, 2 equiv) is added in one portion to a mixture of 4-nitro-m-cresol (1.9 g, 12.59 mMol) and powdered NaOH (0.605 g, 15.11 mMol, 1.2 equiv) in DMF (25 mL). The reaction mixture is stirred for 1 h at rt, diluted with H₂O (300 mL), and extracted with EtOAc (600 mL). The aqueous layer is saturated with NaCl and extracted with CH₂Cl₂/MeOH (9:1, 2×300 mL). The combined organic phase is dried (Na₂SO₄), filtered, and concentrated. The resulting yellow crystalline material is purified by silica gel column chromatography (Hexane→Hexane/EtOAc, 6:1→4:1) to provide the title compound as white crystals: HPLC ^{D}t_{Ret}=4.7; R_{f}=0.17 (Hexane/EtOAc, 3:1).

### Step 74.4: 3′-Chloro-2-trifluoromethyl-biphenyl-4-amine

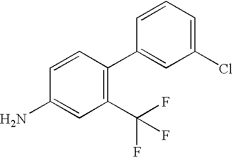

A mixture of 5-amino-2-bromobenzotrifluoride (500 mg, 2.1 mMol), 3-chlorophenylboronic acid (970 mg, 6.2 mMol, 3 equiv), Pd(PPh₃)₄ (70 mg, 0.018 mMol, 0.03 equiv), Na₂CO₃ (2 M solution in H₂O, 5 mL, 10 mMol, 4.76 equiv), and toluene (14 mL) is stirred at reflux for 1 h. The reaction mixture is allowed to cool to rt and filtered through a pad of celite, washing the filter cake with CH₂Cl₂ and H₂O. The layers are separated and the aqueous phase is extracted with CH₂Cl₂ (2×60 mL). The combined organic phase is washed with brine, dried (Na₂SO₄), filtered and concentrated in vacuo. MPLC(CH₃CN/H₂O/TFA) purification of the crude material affords the title compound: MS: 270.0 [M−2]⁻; HPLC ^{D}t_{Ret}=4.9.

### Example 75

### 1-(3′-Bromo-2-trifluoromethyl-biphenyl-4-yl)-3-{4-[2-(4-dimethylamino-butylamino)-pyrimidin-4-yloxy]-2-methyl-phenyl}urea

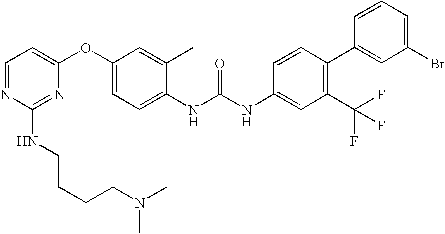

The title compound is prepared as described in Ex. 74 for 1-(3′-chloro-2-trifluoromethyl-biphenyl-4-yl)-3-4-[2-(4-dimethylamino-butylamino)-pyrimidin-4-yloxy]-2-methyl-phenyl)-urea but using 3′-bromo-2-trifluoromethyl-biphenyl-4-amine. The title compound: MS: 658.8 [M+1]⁺; HPLC ^{D}t_{Ret}=4.3; R_{f}=0.47 (CH₂Cl₂/MeOH, 99:1).

### Step 75.1: 3′-Bromo-2-trifluoromethyl-biphenyl-4-amine

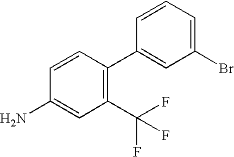

The title compound is prepared as described in Ex. 74 (Step 74.4) for 1-(3′-chloro-2-trifluoromethyl-biphenyl-4-yl)-3-{4-[2-(4-dimethylamino-butylamino)-pyrimidin-4-yloxy]-2-methyl-phenyl}-urea but using 3-bromophenylboronic acid. The title compound: MS: 315.9 [M−1]⁻; HPLC ^{D}t_{Ret}=4.9; R_{f}=0.16 (Hexane/EtOAc, 4:1).

### Example 76

### 1-(4′-Chloro-2-trifluoromethyl-biphenylyl-4-yl)-3-{4-[2-(4-dimethylamino-butylamino)-pyrimidin-4-yloxy]-2-methyl-phenyl}-urea

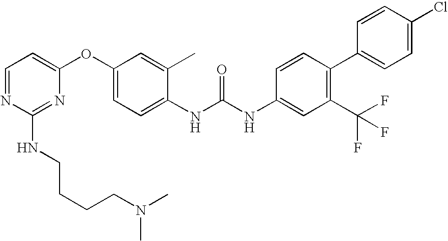

The title compound is prepared as described in Ex. 74 for 1-(3′-chloro-2-trifluoromethyl-biphenyl-4-yl)-3-{4-[2-(4-dimethylamino-butylamino)-pyrimidin-4-yloxy]-2-methyl-phenyl}-urea but using 4′-chloro-2-trifluoromethyl-biphenyl-4-amine. The title compound: MS: 612.9 [M]⁺; HPLC ^{D}t_{Ret}=4.3; R_{f}=0.13 (CH₂Cl₂/MeOH+1% NH₃^{aq}, 9:1).

### Step 76.1: 4′-Chloro-2-trifluoromethyl-biphenyl-4-amine

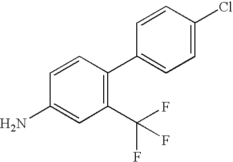

The title compound is prepared as described in Ex. 74 (Step 74.4) for 1-(3′-chloro-2-trifluoromethyl-biphenyl-4-yl)-3-{4-[2-(4-dimethylamino-butylamino)-pyrimidin-4-yloxy]-2-methyl-phenyl}-urea but using 4-chlorophenylboronic acid. The title compound: MS: 270.0 [M−2]⁻; HPLC ^{D}t_{Ret}=4.9.

### Example 77

### 1-(4′-Bromo-2-trifluoromethyl-biphenyl-yl)-3-{4-[2-(4-dimethylamino-butylamino)-pyrimidin-4-yloxy]-2-methyl-phenyl}urea

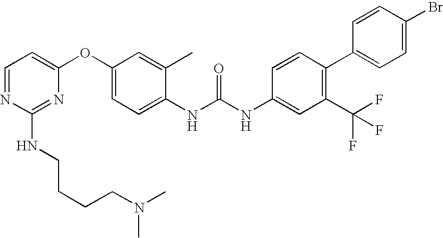

The title compound is prepared as described in Ex. 74 for 1-(3′-chloro-2-trifluoromethyl-biphenyl-4-yl)-3-{4-[2-(4-dimethylamino-butylamino)-pyrimidin-4-yloxy]-2-methyl-phenyl}-urea but using 4′-bromo-2-trifluoromethyl-biphenyl-4-amine. The title compound: MS: 658.8 [M+1]⁺; HPLC ^{D}t_{Ret}=4.4; R_{f}=0.07 (CH₂Cl₂/MeOH+1% NH₃^{aq}, 9:1).

### Step 77.1: 4′-Bromo-2-trifluoromethyl-biphenyl-4-amine

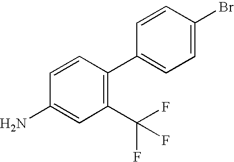

The title compound is prepared as described in Ex. 74 (Step 74.4) for 1-(3′-chloro-2-trifluoromethyl-biphenyl-4-yl)-3-{4-[2-(4-dimethylamino-butylamino)-pyrimidin-4-yloxy]-2-methyl-phenyl}-urea but using 4′-bromophenylboronic acid. The title compound: MS: 315.9 [M−1]⁻; HPLC ^{D}t_{Ret}=4.9; R_{f}=0.14 (Hexane/EtOAc, 4:1).

### Example 78

### 1-(3′-Chloro-2-trifluoromethyl-biphenyl-4-yl)-3-{4-[2-(4-dimethylamino-butylamino)-pyrimidin-4-yloxy]-2-trifluoromethyl-phenyl}-urea

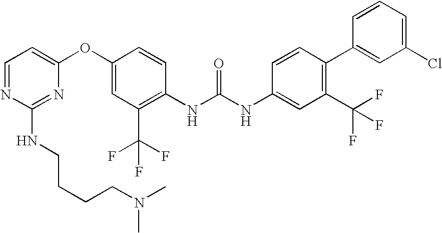

The title compound is prepared as described in Ex. 74 for 1-(3′-chloro-2-trifluoromethyl-biphenyl-4-yl)-3-{4-[2-(4-dimethylamino-butylamino)-pyrimidin-4-yloxy]-2-methyl-phenyl}-urea but using N-[4-(4-amino-3-trifluoromethyl-phenoxy)-pyrimidin-2-yl]-N′,N′-dimethyl-butane-1,4-diamine. The title compound: MS: 668.8 [M+1]⁺; HPLC ^{D}t_{Ret}=4.4; R_{f}=0.01 (CH₂Cl₂/MeOH+1% NH₃^{aq}, 9:1).

### Step 78.1: N-[4-(4-Amino-3-trifluoromethyl-phenoxy)-pyrimidin-2-yl]-N′,N′-dimethyl-butane-1,4-diamine

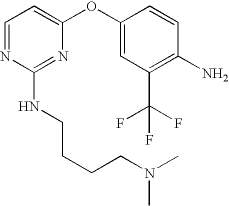

The title compound is prepared as described in Ex. 74 (Step 74.1) for N-[4-(4-amino-3-methyl-phenoxy)-pyrimidin-2-yl]-N′,N′-dimethyl-butane-1,4-diamine but using [4-(2-chloropyrimidin-4-yloxy)-2-trifluoromethyl-phenylamine. The title compound: MS: 370.1 [M]⁺; HPLC ^{D}t_{Ret}=2.6; R_{f}=0.14 (CH₂Cl₂/MeOH+1% NH₃^{aq}, 4:1).

### Step 78.2: [4-(2-Chloro-pyrimidin-4-yloxy)-2-trifluoromethyl-phenylamine

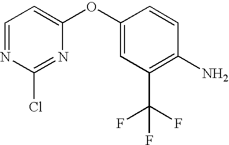

The title compound is prepared as described in Ex. 74 (Step 74.2) for [4-(2-chloro-pyrimidin-4-yloxy)-2-methyl-phenylamine but using 2-chloro-4-(4-nitro-3-trifluoromethyl-phenoxy)-pyrimidine. The title compound: MS: 288.0 [M−1]⁻; HPLC ^{D}t_{Ret}=4.6.

### Step 78.3: 2-Chloro-4-(4-nitro-3-trifluoromethyl-phenoxy)-pyrimidine

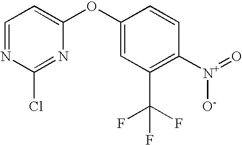

The title compound is prepared as described in Ex. 74 (Step 74.3) for 2-chloro-4-(3-methyl-4-nitro-phenoxy)-pyrimidine but using 4-nitro-3-(trifluoromethyl)-phenol. The reaction mixture is stirred for 3 h at rt. The title compound: MS: 317.9 [M−1]⁻; HPLC ^{D}t_{Ref}=4.8.

### Example 79

### 1-(3′-Bromo-2-trifluoromethyl-biphenyl-4-yl)-3-{4-[2-(4-dimethylamino-butylamino)-pyrimidin-4-yloxy]-2-trifluoromethyl-phenyl}-urea

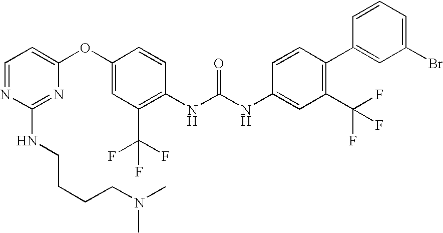

The title compound is prepared as described in Ex. 75 for 1-(3′-bromo-2-trifluoromethyl-biphenyl-yl)-3-{4-[2-(4-dimethylamido-butylamino)-pyrimidin-4-yloxy]-2-methyl-phenyl}-urea but using N-[4-(4-amino-3-trifluoromethyl-phenoxy)-pyrimidin-2-yl]-N′,N′-dimethyl-butane-1,4-diamine (Ex. 78, Step 78.1). The title compound: MS: 712.7 [M+1]4; HPLC ^{D}t_{Ret}=4.5; R_{f} 0.04 (CH₂Cl₂/MeOH+1% NH₃^{aq}, 9:1).

### Example 80

### 1-(4′-Chloro-2-trifluoromethyl-biphenyl-4-yl)-3-{4-[2-(4-dimethylamino-butylamino)-pyrimidin-4-yloxy]-2-trifluoromethyl-phenyl}-urea

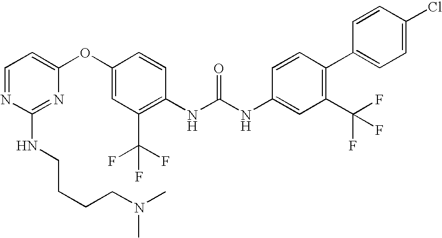

The title compound is prepared as described in Ex. 76 for 1-(4′-chloro-2-trifluoromethyl-biphenyl-4-yl)-3-{4-[2-(4-dimethylamino-butylamino)-pyrimidin-4-yloxy]-2-methyl-phenyl}-urea but using N-[4-(4-amino-3-trifluoromethyl-phenoxy)-pyrimidin-2-yl]-N′,N′-dimethyl-butane-1,4-diamine (Ex. 78, Step 78.1). The title compound: MS: 668.8 [M+1]⁺; HPLC ^{D}t_{Ret}=4.5; R_{f}=0.08 (CH₂Cl₂/MeOH+1% NH₃^{aq}, 9:1).

### Example 81

### 1-(4′-Bromo-2-trifluoromethyl-biphenyl-4-yl)-3-{4-[2-(4-dimethylamino-butylamino)-pyrimidin-4-yloxy]-2-trifluoromethyl-Phenyl}-urea

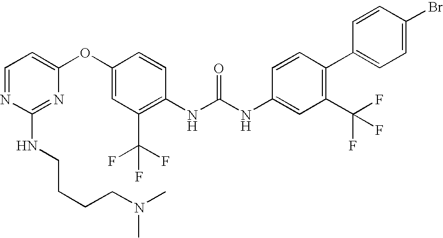

The title compound is prepared as described in Ex. 77 for 1-(4′-bromo-2-trifluoromethyl-biphenyl-4-yl)-3-{4-[2-(4-dimethylamino-butylamino)-pyrimidin-4-yloxy]-2-methyl-phenyl}-urea but using N-[4-(4-amino-3-trifluoromethyl-phenoxy)-pyrimidin-2-yl]-N′,N′-dimethyl-butane-1,4-diamine (Ex. 78, Step 78.1). The title compound: MS: 712.7 [M+1]⁺; HPLC ^{D}t_{Ret}=4.5; R_{f}=0.07 (CH₂Cl₂/MeOH+1% NH₃^{aq}, 9:1).

### Example 82

### 1-(3-Chloro-2-trifluoromethyl-biphenyl-4-yl)-3-{4-[2-(4-dimethylamino-butylamino)-pyrimidin-4-yloxy]-phenyl}-urea

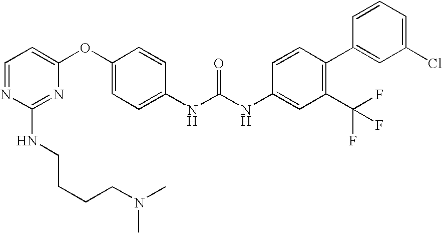

The title compound is prepared as described in Ex. 74 for 1-(3′-chloro-2-trifluoromethyl-biphenyl-4-yl)-3-{4-[2-(4-dimethylamino-butylamino)-pyrimidin-4-yloxy]-2-methyl-phenyl}-urea but using N-[4-(4-amino-phenoxy)-pyrimidin-2-yl]-N′,N′-dimethyl-butane-1,4-diamine. The title compound: MS: 600.9 [M+1]⁺; HPLC ^{D}t_{Ret}=4.3; R_{f}=0.02 (CH₂Cl₂/MeOH+1% NH₃^{aq}, 9:1).

### Step 82.1: N-[4-(4-Amino-phenoxy)-pyrimidin-2-yl]-N′,N′-dimethyl-butane-1,4-diamine

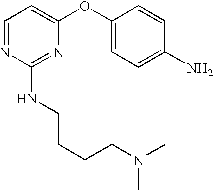

The title compound is prepared as described in Ex. 74 (Step 74.1) for N-[4-(4-amino-3-methyl-phenoxy)-pyrimidin-2-yl]-N′,N′-dimethyl-butane-1,4-diamine but using 4-(2-chloropyrimidin-4-yloxy)-phenylamine. The title compound: MS: 302.2 [M]⁺; R_{f}=0.27 (CH₂Cl₂/MeOH+1% NH₃^{aq}, 4:1).

### Step 82.2: 4-(2-Chloro-pyrimidin-4-yloxy)-phenylamine

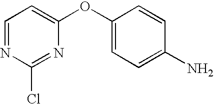

The title compound is prepared as described in Ex. 74 (Step 74.2) for [4-(2-chloro-pyrimidin-4-yloxy)-2-methyl-phenylamine but using 2-chloro-4-(4-nitro-phenoxy)-pyrimidine (Ex. 45, Step 45.1). The title compound: MS: 223.9 [M+1]⁻; HPLC ^{D}t_{Ret}=1.6; R_{f} 0.62-(CH₂Cl₂/MeOH, 95:5).

### Example 83

### 1-(4′-Chloro-2-trifluoromethyl-biphenyl-4-yl)-3-{4-[2-(4-dimethylamino-butylamino)-pyrimidin-4-yloxy]-phenyl}-urea

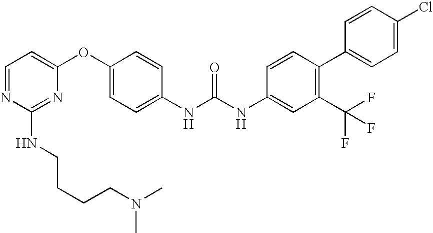

The title compound is prepared as described in Ex. 76 for 1-(4′-chloro-2-trifluoromethyl-biphenyl-4-yl)-3-{4-[2-(4-dimethylamino-butylamino)-pyrimidin-4-yloxy]-2-methyl-phenyl}urea but using N-[4-(4-amino-phenoxy)-pyrimidin-2-yl]-N′,N′-dimethyl-butane-1,4-diamine (Ex. 82, Step 82.1). The title compound: MS: 598.9 [M]⁺; HPLC ^{D}t_{Ret}=4.3; R_{f}=0.10 (CH₂Cl₂/MeOH+1% NH₃^{aq}, 9:1).

### Example 84

### 1-(4′-Bromo-2-trifluoromethyl-biphenyl-4-yl)-3-{4-[2-(4-dimethylamino-butylamino)-pyrimidin-4-yloxy]-phenyl}-urea

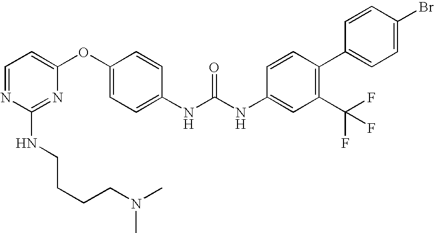

The title compound is prepared as described in Ex. 77 for 1-(4′-bromo-2-trifluoromethyl-biphenyl-4-yl)-3-{4-[2-(4-dimethylamino-butylamino)-pyrimidin-4-yloxy]-2-methyl-phenyl}-urea but using N-[4-(4-amino-phenoxy)-pyrimidin-2-yl]-N′,N′-dimethyl-butane-1,4-diamine (Ex. 82, Step 82.1). The title compound: MS: 644.8 [M+1]⁺; HPLC ^{D}t_{Ret}=4.3; R_{f}=0.10 (CH₂Cl₂/MeOH+1% NH₃^{aq}, 9:1).

### Example 85

### 1-{4-[2-(3-Methoxy-phenylamino)-pyrimidin-4-yloxy]-phenyl}-3-[4-(4-methylpiperazin-1-ylmethyl)-3-trifluoromethyl-phenyl]-urea

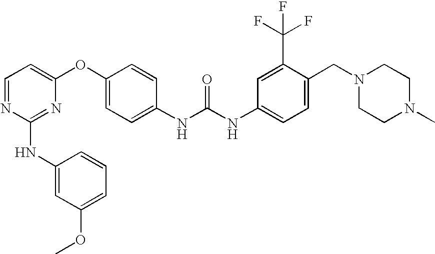

The title compound is prepared as described in Ex. 74 for 1-(3′-chloro-2-trifluoromethyl-biphenyl-4-yl)-3-{4-[2-(4-dimethylamino-butylamino)-pyrimidin-4-yloxy]-2-methyl-phenyl}-urea but using [4-(4-Amino-3-methyl-phenoxy)-pyrimidin-2-yl]-(3-methoxy-phenyl)-amine and 4-(4-methylpiperazin-1-ylmethyl)-3-trifluoromethyl-aniline (Ex. 14, Step 14.4). The title compound: MS: 622.0 [M+1]⁺; HPLC ^{D}t_{Ret}=3.5; R_{f}=0.33 (CH₂Cl₂/MeOH+1% NH₃^{aq}, 9:1).

### Step 85.1: [4-(4-Amino-3-methyl-phenoxy)-pyrimidin-2-yl]-(3-methoxy-phenyl)-amine

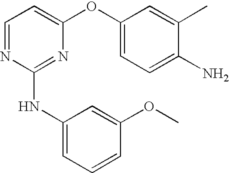

A mixture of (3-methoxy-phenyl)-[4-(3-methyl-4-nitro-phenoxy)-pyrimidin-2-yl]-amine (400 mg, 1.14 mMol) and Raney-Ni (200 mg) in MeOH/THF (3:1, 40 mL) is stirred for 2 h at rt, under a hydrogen atmosphere. The reaction mixture is filtered through a pad of celite and the filtrate is concentrated in vacuo to afford the title compound as a yellow-brown solid: MS: 323.1 [M+1]⁺; HPLC ^{D}t_{Ret}=2.6.

### Step 85.2: (3-Methoxy-phenyl)-[4-(3-methyl-4-nitro-phenoxy)-pyrimidin-2-yl]-amine

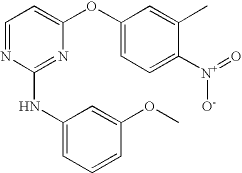

A mixture of 2-chloro-4-(3-methyl-4-nitro-phenoxy)-pyrimidine (Ex. 74, Step 74.3) (700 mg, 2.63 mMol), m-anisidine (357 mg, 2.90 mMol, 1.1 equiv), and 2-propanol (10.5 mL) is stirred for 1 h at 100° C. The reaction mixture is allowed to cool to rt, diluted with H₂O (90 mL) and extracted with CH₂Cl₂ (350 mL). The organic phase is washed with brine, dried (Na₂SO₄), filtered and concentrated. The title compound: MS: 353.3 [M+1]⁺; HPLC ^{D}t_{Ret}=4.6; R_{f}=0.08 (Hexane/EtOAc; 3:1).

### Example 86

### 1-2-Methyl-4-{2-[4-(4-methyl-piperazin-1-yl)-phenylamino]-pyrimidin-4-yloxy}-phenyl)-3-(3-trifluoromethyl-phenyl)-urea

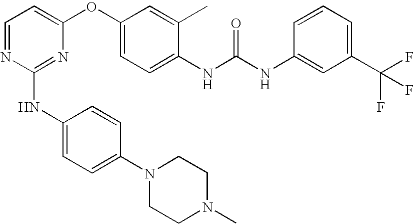

The title compound is prepared as described in Ex. 74 for 1-(3′-chloro-2-trifluoromethyl-biphenyl-4-yl)-3-{4-[2-(4-dimethylamino-butylamino)-pyrimidin-4-yloxy]-2-methyl-phenyl}-urea but using [4-(4-amino-3-methyl-phenoxy)-pyrimidin-2-yl]-[4-(4-methyl-piperazin-1-yl)-phenyl]-amine and 3-aminobenzotrifluoride. The title compound: MS: 577.9 [M]⁺; HPLC ^{D}t_{Ret}=3.7; R_{f}=0.29 (CH₂Cl₂/MeOH, 9:1).

### Step 86.1: [4-(4-Amino-3-methyl-phenoxy)-pyrimidin-2-yl]-[4-(4-methyl-piperazin-1-yl) -phenyl]-amine

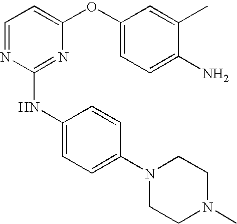

A mixture of [4-(3-methyl-4-nitro-phenoxy)-pyrimidin-2-yl]-[4-(4-methyl-piperazin-1-yl)-phenyl]-amine (133 mg. 0.32 mMol) and Raney-M (50 mg) in MeOH (10 mL) is stirred for 6 h at rt, under a hydrogen atmosphere. The reaction mixture is filtered through a pad of celite and the filtrate is concentrated in vacuo to afford the title compound as a red-brown solid: MS: 391.1 [M]⁺; HPLC ^{D}t_{Ret}=1.3.

### Step 86.2: [4-(3-Methyl-4-nitro-phenoxy)-pyrimidin-2-yl]-[4-(4-methyl-piperazin-1-yl)-phenyl]-amine

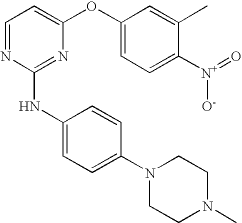

A mixture of 2-chloro-4-(3-methyl-4-nitro-phenoxy)-pyrimidine (Ex. 74, Step 74.3) (400 mg, 1.51 mMol), 4-(4-methyl-piperazin-1-yl)-phenylamine (318 mg, 1.66 mMol, 1.1 equiv), 4 N HCl (1.1 mL, 4.08 mMol, 2.7 equiv), and 2-propanol (6 mL) is stirred for 1 h at 100° C. The reaction mixture is allowed to cool to rt, diluted with H₂O (30 mL) and extracted with CH₂Cl₂ (120 mL). The organic phase is washed with brine, dried (Na₂SO₄), filtered and concentrated. The title compound: MS: 421.1 [M+1]⁺; HPLC ^{D}t_{Ret}=3.1; R_{f}=0.39 (CH₂Cl₂/MeOH, 9:1).

### Example 87

### 1-{4-[6-(5-Chloro-2-methoxy-phenylamino)-pyrimidin-4-yloxy]-phenyl}-3-(4-morpholin-4-yl-3-trifluoromethyl-phenyl)-urea

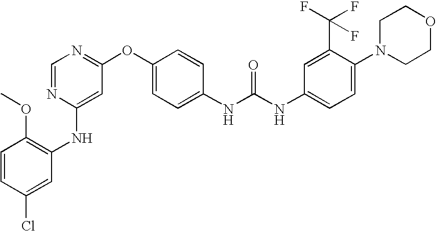

To a solution of 1-[4-(6-chloro-pyrimidin-4-yloxy)-phenyl]-3-(4-morpholin-4-yl-3-trifluoromethyl-phenyl)-urea (Step 69.1) (34 mg, 68 μmol) in 3 ml of isopropanol:dioxane (1:1, v/v) is added 5-chloro-2-methoxy-phenylamine (54 mg, 340 μmol; Fluka, Buchs, Switzerland) and HCl conc. (5 μl). The mixture is heated in a microwave oven (Emrys Optimizer, Personal Chemistry; Uppsala, Sweden) until completion of the reaction. The reaction mixture is diluted with EtOAc (50 ml) and extracted with 0.1 N NaOH (×2) and water (×2). The water phases are discarded, and the organic one is dried (Na₂SO₄), filtered and concentrated to dryness. The title compound is obtained by chromatography on silica gel (CH₂Cl₂:MeOH, 98:2, v/v): MS: 615.2, 616.4, 617.4; HPLC t_{Ret}^{new}=8.67 (NEW GRADIENT: Linear gradient over 7 min of MeCN/0.09% TFA and H₂O/0.1% TFA from 1:49 to 1:0 and 3 min at 1:0, detection at 215 nm, flow rate 2.0 ml/min. Column: Nucleosil C₁₈-column (250×4.6 mm, 5 μm, 100 Å).

The following compounds are prepared as described in Example 87 using the appropriate amine derivative:

| | | ES-MS | tᵣₑₜ^{new} |
|---|---|---|---|
| Example | Compound name | (M + H)⁺ | [min] |
| 88 | 1-{4-[6-(4-Methyl-piperazin-1-yl)-pyrimidin-4-yloxy]- | 558.2 | 6.69 |
| | phenyl}-3-(4-morpholin-4-yl-3-trifluoromethyl-phenyl)-urea | | |
| 89 | 1-[4-(6-Dimethylamino-pyrimidin-4-yloxy)-phenyl]-3-(4- | 503.3 | 7.14 |
| | morpholin-4-yl-3-trifluoromethyl-phenyl)-urea | | |
| 90 | N,N-Dimethyl-4-(6-{4-[3-(4-morpholin-4-yl-3- | 622.4 | 7.68 |
| | trifluoromethyl-phenyl)-ureido]-phenoxy}-pyrimidin-4- | | |
| | ylamino)-benzamide | | |
| 91 | 1-{4-[6-(2-Methoxy-5-methyl-phenylamino)-pyrimidin-4- | 595.6 | 8.17 |
| | yloxy]-phenyl}-3-(4-morpholin-4-yl-3-trifluoromethyl- | | |
| | phenyl)-urea | | |
| 92 | 1-{4-[6-(2-Methoxy-5-nitro-phenylamino)-pyrimidin-4- | 626.5 | 8.50 |
| | yloxy]-phenyl}-3-(4-morpholin-4-yl-3-trifluoromethyl- | | |
| | phenyl)-urea | | |
| 93 | 1-{4-[6-(2,5-Dimethoxy-phenylamino)-pyrimidin-4-yloxy]- | 611.5 | 8.10 |
| | phenyl}-3-(4-morpholin-4-yl-3-trifluoromethyl- | | |
| | phenyl)-urea | | |
| 94 | N,N-Diethyl-4-methoxy-3-(6-{4-[3-(4-morpholin-4-yl-3- | 716.4 | 8.39 |
| | trifluoromethyl-phenyl)-ureido]-phenoxy}-pyrimidin-4- | | |
| | ylamino)-benzenesulfonamide | | |
| 95 | 1-{4-[6-(2-Methoxy-phenylamino)-pyrimidin-4-yloxy]- | 581.3 | 7.91 |
| | phenyl}-3-(4-morpholin-4-yl-3-trifluoromethyl-phenyl)- | | |
| | urea | | |

### Example 96

### Inhibition of the Protein Tyrosine Kinase Activity of Ret

The inhibition tests are carried out as described above. The IC₅₀ values for some of the compounds of formula I are given in the table below:

| Compound Name | IC₅₀ RET [μM] |
|---|---|
| 1-[4-(6-Amino-pyrimidin-4-yloxy)-phenyl]-3-(3-azetidin- | 0.083 |
| 1-ylmethyl-5-trifluoromethyl-phenyl)-urea | |
| 1-(3-Dimethylaminomethyl-5-trifluoromethyl-phenyl)-3- | 0.11 |
| [4-(6-methylamino-pyrimidin-4-yloxy)-phenyl]-urea | |
| 1-[4-(2-Amino-pyrimidin-4-yloxy)-phenyl]-3-[4-(4- | 0.18 |
| methyl-piperazin-1-ylmethyl)-3-trifluoromethyl-phenyl]- | |
| urea | |
| 1-[4-(6-Amino-pyrimidin-4-yloxy)-phenyl]-3-[3-(4- | 0.26 |
| methyl-piperazin-1-ylmethyl)-5-trifluoromethyl-phenyl]- | |
| urea | |
| 1-[4-(2-Amino-pyrimidin-4-yloxy)-phenyl]-3-[4-(4-ethyl- | 0.31 |
| piperazin-1-ylmethyl)-3-trifluoromethyl-phenyl]-urea | |
| 1-[4-(4-Ethyl-piperazin-1-ylmethyl)-3-trifluoromethyl- | 0.35 |
| phenyl]-3-[4-(2-methylamino-pyrimidin-4-yloxy)- | |
| phenyl]-urea | |
| 1-[4-(2-Amino-pyrimidin-4-yloxy)-phenyl]-3-[4-(4- | 0.4 |
| isopropyl-piperazin-1-ylmethyl)-3-trifluoromethyl- | |
| phenyl]-urea | |
| 1-[4-(2-Methylamino-pyrimidin-4-yloxy)-phenyl]-3-[4- | 0.45 |
| (4-methyl-piperazin-1-yl)-3-trifluoromethyl-phenyl]-urea | |
| 1-[4-(2-Methylamino-pyrimidin-4-yloxy)-phenyl]-3-[3- | 0.45 |
| (4-methyl-piperazin-1-yl)-5-trifluoromethyl-phenyl]-urea | |
| 1-[4-(6-Amino-pyrimidin-4-yloxy)-phenyl]-3-[4-(4- | 0.55 |
| isopropyl-piperazin-1-ylmethyl)-3-trifluoromethyl- | |
| phenyl]-urea | |
| 1-[4-(2-Amino-pyrimidin-4-yloxy)-phenyl]-3-[4-(4-tert- | 0.56 |
| butyl-piperazin-1-ylmethyl)-3-trifluoromethyl-phenyl]- | |
| urea | |
| 1-[4-(4-Ethyl-piperazin-1-ylmethyl)-3-trifluoromethyl- | 0.58 |
| phenyl]-3-[4-(6-methylamino-pyrimidin-4-yloxy)- | |
| phenyl]-urea | |
| 1-[4-(6-Methylamino-pyrimidin-4-yloxy)-phenyl]-3-[3- | 0.59 |
| (4-methyl-piperazin-1-ylmethyl)-5-trifluoromethyl- | |
| phenyl]-urea | |
| 1-[4-(2-Amino-pyrimidin-4-yloxy)-phenyl]-3-(4-(4,5- | 0.85 |
| dimethyl-imidazol-1-ylmethyl)-3-trifluoromethyl- | |
| phenyl]-urea | |
| 1-[3-(4-Isopropyl-piperazin-1-ylmethyl)-5- | 0.96 |
| trifluoromethyl-phenyl]-3-[4-(6-methylamino-pyrimidin- | |
| 4-yloxy)-phenyl]-urea | |

### Example 97

### Inhibition of the Protein Tyrosine Kinase Activity of Flt-3

The inhibition tests are carried out as described above. The IC₅₀ values for some of the compounds of the Examples are given in the table below:

| Example | IC₅₀ Flt-3 | Example | IC₅₀ Flt-3 | Example | IC₅₀ Flt-3 |
|---|---|---|---|---|---|
| No. | [μM] | No. | [μM] | No. | [μM] |
| 1 | 0.905 | 34d.1 | 0.67 | 51a.1 | 0.085 |
| 2 | 1.2 | 34d.3 | 0.29 | 51a.2 | 0.12 |
| 4 | 0.153 | 34e.1 | 0.16 | 51b.1 | 0.13 |
| 5 | 0.54 | 34e.3 | 0.079 | 51b.2 | 0.17 |
| 6 | 0.4 | 34g.1 | 0.3 | 51d.1 | 0.091 |
| 8 | 0.51 | 34g.3 | 0.378 | 51d.2 | 0.135 |
| 9 | 0.32 | 34j.1 | 0.25 | 51e.1 | 0.25 |
| 11 | 0.23 | 34j.3 | 0.283 | 51e.2 | 0.91 |
| 13 | 0.34 | 34k.1 | 0.13 | 52a | 0.12 |
| 14 | 0.36 | 34k.3 | 0.1 | 52b | 0.08 |
| 15 | 0.6 | 34l.1 | 0.62 | 52c | 0.029 |
| 16 | 0.36 | 34m.1 | 0.4 | 52d | 0.26 |
| 17 | 0.94 | 34m.3 | 0.2 | 53b | 0.12 |
| 19 | 0.25 | 34n.1 | 0.31 | 53d | 0.19 |
| 19-1 | 0.038 | 34n.3 | 0.2 | 55c | 0.37 |
| 19-2 | 0.08 | 34p.1 | 0.59 | 55d | 0.97 |
| 21 | 1.8 | 34s.2 | 0.24 | 57 | 0.118 |
| 23 | 1.3 | 34t.2 | 0.29 | 58 | 0.12 |
| 24 | 0.17 | 34u.2 | 1.5 | 59 | 0.076 |
| 34a.1 | 1.1 | 34w.2 | 0.14 | 60 | 0.16 |
| 34a.3 | 0.83 | 38 | 0.354 | 61 | 0.49 |
| 34b.1 | 0.36 | 41 | 0.42 | 62 | 0.16 |
| 34b.3 | 0.37 | 43 | 0.16 | 63 | 0.14 |
| 34c.1 | 0.54 | 48 | 0.58 | 64 | 0.34 |
| 34c.3 | 0.35 | 50 | 0.12 | | |

### Example 98

### Inhibition of Flt-3 Dependent Cell Proliferation

The inhibition assay is carried out as described above using the wild type IL-3-dependent hematopoietic cell line Ba/F3 and the mutant sub-lines ITD-Ba/F3 or D835Y-Ba/F3 expressing constitutively activating Flt-3 kinases. The ED₅₀ values for some of the compounds of the Examples are given in the table below:

Inhibition of Flt-3
dependent Proliferation
(ED₅₀ [nM])

| Example No. | ITD-mutant | D835-mutant |
|---|---|---|
| 53c | 0.1 | 3.3 |
| 55a | <0.5 | <0.5 |
| 45 | <0.2 | 0.5 |
| 46 | <0.2 | 3.9 |
| 55b | <0.5 | <0.5 |
| 49 | 0.1 | 11.7 |
| 53a | <0.5 | 1.0 |

### Example 99

### Tablets Comprising a Compound of the Examples

Tablets, comprising, as active ingredient, 100 mg of any one of the compounds of Examples 1 to 95 are prepared with the following composition, following standard procedures:

| Composition | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, Stempeldurchmesser 10 mm).

Avicel is microcrystalline cellulose (FMC, Philadelphia, USA).

PVPPXL is polyvinylpolypyrrolidone, cross-linked (BASF, Germany).

Aerosil is silcium dioxide (Degussa, Germany).

### Example 100

### Capsules

Capsules, comprising, as active ingredient; 100 mg of any one of the compounds of Examples 1 to 95, of the following composition are prepared according to standard procedures:

| Composition | | |
|---|---|---|
| Active Ingredient | 100 | mg |
| Avicel | 200 | mg |
| PVPPXL | 15 | mg |
| Aerosil | 2 | mg |
| magnesium stearate | 1.5 | mg |
| | 318.5 | mg |

Manufacturing is done by mixing the components and filling them into hard gelatine capsules, size 1.

What is claimed is:

1-12. (canceled)

13. A method of treating RET dependent diseases comprising administering compound of formula I 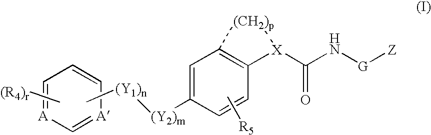
wherein G is either not present, lower alkylene or C₃-C₅cycloalkylene and Z is a radical of the formula Ia 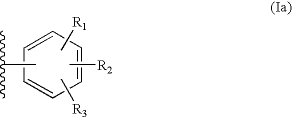
or G is not present and Z is a radical of the formula Ib 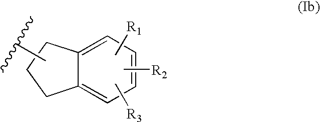
A is CH, N or N→O and A′ is N or N→O, with the proviso that not more than one of A and A′ can be N→O;
n is 1 or 2;
m is 0, 1 or 2;
p is 0, 2 or 3;
r is 0 to 5;
X is NR if p is 0, wherein R is hydrogen or an organic moiety, or if p is 2 or 3, X is nitrogen which together with (CH₂)ₚ and the bonds represented in dotted (interrupted) lines (including the atoms to which they are bound) forms a ring,
or
X is CHK wherein K is lower alkyl or hydrogen and p is zero,
with the proviso that the bonds represented in dotted lines, if p is zero, are absent;
Y₁ is O, S or CH₂;
Y₂ is O, S or NH;
with the proviso that (Y₁)ₙ—(Y₂)ₘ does not include O—O, S—S, NH—O, NH—S or S—O groups;
each of R₁, R₂, R₃ and R₅, independently of the others, is hydrogen or an inorganic or organic moiety or any two of them together form a lower alkylene-dioxy bridge bound via the oxygen atoms, and the remaining one of these moieties is hydrogen or an inorganic or organic moiety;
and R₄ (if present, that is, if r is not zero) is an inorganic or organic moiety;
or a tautomer thereof;
or a pharmaceutically acceptable salt thereof.

14. The method according to claim 13, wherein the RET dependent disease is a RET dependent tumour disease.

15. The method according to claim 14, wherein the RET dependent tumour disease is selected from colon cancer, lung cancer, breast cancer, pancreatic cancer and thyroid cancer.

16. The method according to claim 15, wherein the cancer is thyroid cancer.

17. An N-[4-(pyrimidin-4-yloxy)-phenyl]-N′-phenyl-urea derivative selected from the group consisting of the compounds of Examples 34, 51-53, 55, 56 and 72 as described in the description, or a salt thereof.

18. An N-[4-(pyrimidin-4-yloxy)-phenyl]-N′-phenyl-urea derivative selected from the group consisting of:
N-[4-(6-Chloro-pyrimidin-4-yloxy)-phenyl]-N′-[3-(azetidin-1-ylmethyl)-5-trifluoromethyl-phenyl]-urea;
N-[4-(6-Methylamino-pyrimidin-4-yloxy)-phenyl]-N′-(3-azetidin-1-ylmethyl-5-trifluoromethyl-phenyl)-urea;
N-[4-(6-Azido-pyrimidin-4-yloxy)-phenyl]-N′-(3-azetidin-1-ylmethyl-5-trifluoromethyl-phenyl)-urea;
N-[4-(6-Amino-pyrimidin-4-yloxy)-phenyl]-N′-(3-azetidin-1-ylmethyl-5-trifluoromethyl-phenyl)-urea;
N-[4-(6-Chloro-pyrimidin-4-yloxy)-phenyl]-N′-[3-(4-isopropylpiperazin-1-ylmethyl)-5-trifluoromethyl-phenyl]-urea;
N-[4-(6-Methylamino-pyrimidin-4-yloxy)-phenyl]-N′-[3-(4-isopropylpiperazin-1-ylmethyl)-5-trifluoromethyl-phenyl]-urea;
N-[4-(6-Azido-pyrimidin-4-yloxy)-phenyl]-N′-[3-(4-isopropylpiperazin-1-ylmethyl)-5-trifluoromethyl-phenyl]-urea;
N-[4-(6-Amino-pyrimidin-4-yloxy)-phenyl]-N′-[3-(4-isopropylpiperazin-1-ylmethyl)-5-trifluoromethyl-phenyl]-urea;
N-[4-(6-Chloro-pyrimidin-4-yloxy)-phenyl]-N′-[3-(4-methylpiperazin-1-ylmethyl)-5-trifluoromethyl-phenyl]-urea;
N-[4-(6-Chloro-pyrimidin-4-yloxy)-phenyl]-N′-[(3-diethylaminomethyl-5-trifluoromethyl-phenyl]-urea;
N-[4-(6-Methylamino-pyrimidin-4-yloxy)-phenyl]-N′-(3-diethylaminomethyl-5-trifluoromethyl-phenyl)-urea;
N-[4-(6-Azido-pyrimidin-4-yloxy)-phenyl]-N′-(3-diethylaminomethyl-5-trifluoromethyl-phenyl)-urea;
N-[4-(6-Amino-pyrimidin-4-yloxy)-phenyl]-N′-(3-diethylaminomethyl-5-trifluoromethyl-phenyl)-urea;
N-[4-(6-Chloro-pyrimidin-4-yloxy)-phenyl]-N′-[4-(4-methylpiperazin-1-ylmethyl)-3-trifluoromethyl-phenyl]-urea;
N-[4-(6-Chloro-pyrimidin-4-yloxy)-phenyl]-N′-[4-(4-isopropylpiperazin-1-ylmethyl)-3-trifluoromethyl-phenyl]-urea;
N-[4-(6-Methylamino-pyrimidin-4-yloxy)-phenyl]-N′-[4-(4-isopropylpiperazin-1-ylmethyl)-3-trifluoromethyl-phenyl]-urea trifluoroacetate;
N-[4-(6-Methylamino-pyrimidin-4-yloxy)-phenyl]-N′-[4-(4-isopropyl-4-oxy-piperazin-1-ylmethyl)-3-trifluoromethyl-phenyl]-urea trifluoroacetate;
N-[4-(6-Azido-pyrimidin-4-yloxy)-phenyl]-N′-[4-(4-isopropylpiperazin-1-ylmethyl)-3-trifluoromethyl-phenyl]-urea;
N-[4-(6-Amino-pyrimidin-4-yloxy)-phenyl]-N′-[4-(4-isopropylpiperazin-1-ylmethyl)-3-trifluoromethyl-phenyl]-urea;
N-[4-(6-Methylamino-pyrimidin-4-yloxy)-phenyl]-N′-[4-(4-H-piperazin-1-ylmethyl)-5-trifluoromethyl-phenyl]-urea;
N-[4-(6-Chloro-pyrimidin-4-yloxy)-phenyl]-N′-[4-(4-^{tert}butylpiperazin-1-ylmethyl)-3-trifluoromethyl-phenyl]-urea;
N-[4-(6-Chloro-pyrimidin-4-yloxy)-phenyl]-N′-[4-(4-benzoyloxycarbonyl-piperazin-1-ylmethyl)-3-trifluoromethyl-phenyl]-urea;
N-[4-(6-Chloro-pyrimidin-4-yloxy)-phenyl]-N′-[4-(N,N-dimethylamino-methyl)-3-trifluoromethyl-phenyl]-urea;
N-[4-(6-Chloro-pyrimidin-4-yloxy)-phenyl]-N′-[4-(N,N-diethylamino-methyl)-3-trifluoromethyl-phenyl]-urea;
N-[4-(6-Chloro-pyrimidin-4-yloxy)-phenyl]-N′-[4-[(3-dimethylamino-propyl)-methyl-aminomethyl)]-3-trifluoromethyl-phenyl]-urea;
N-[4-(6-Chloro-pyrimidin-4-yloxy)-phenyl]-N′-[4-[(4-cyano-benzyl)-amino-methyl]-3-trifluoromethyl-phenyl]-urea;
N-[4-(6-Chloro-pyrimidin-4-yloxy)-phenyl]-N′-[4-(1-morpholinyl)-3-trifluoromethyl-phenyl]-urea;
N-[4-(6-Chloro-pyrimidin-4-yloxy)-phenyl]-N′-[4-(pyrrolidin 1-yl-amino-methyl)-3-trifluoromethyl-phenyl]-urea;
N-[4-(6-Chloro-pyrimidin-4-yloxy)-phenyl]-N′-[4-(4-(4-methoxybenzyl)-piperazin-1-ylmethyl)-3-trifluoromethyl-phenyl]-urea;
N-[4-(6-Chloro-pyrimidin-4-yloxy)-phenyl]-N′-[4-(methyl-^{tert}butyl-amino-methyl)-3-trifluoromethyl-phenyl]-urea;
N-[4-(6-Chloro-pyrimidin-4-yloxy)-phenyl]-N′-[4-(azetidin-1-ylmethyl)-3-trifluoromethyl-phenyl]-urea;
N-[4-(6-Chloro-pyrimidin-4-yloxy)-phenyl]-N′-[4-(4,5-dimethylimidazol-1-ylmethyl)-3-trifluoromethyl-phenyl]-urea;
N-[4-(6-Chloro-pyrimidin-4-yloxy)-phenyl]-N′-[4-(2-methylimidazol-1-ylmethyl)-3-trifluoromethyl-phenyl]-urea;
N-[4-(6-Chloro-pyrimidin-4-yloxy)-phenyl]-N′-[4-(2,4-dimethylimidazol-1-ylmethyl)-3-trifluoromethyl-phenyl]-urea;
N-[4-(6-Chloro-pyrimidin-4-yloxy)-phenyl]-N′-[4-(4-ethylpiperazin-1-ylmethyl)-3-methyl-phenyl]-urea;
N-[4-(6-Chloro-pyrimidin-4-yloxy)-phenyl]-N′-[4-(4-ethylpiperazin-1-ylmethyl)-phenyl]-urea;
1-(4-[1,4′]Bipiperidinyl-1′-yl-3-trifluoromethyl-phenyl)-3-[4-(6-chloro-pyrimidin-4-yloxy)-phenyl]-urea;
1-[4-(6-Chloro-pyrimidin-4-yloxy)-phenyl]-3-{4-[4-(2,2-dimethyl-propyl)-piperazin-1-ylmethyl]-3-trifluoromethyl-phenyl}-urea;
1-[4-(6-Chloro-pyrimidin-4-yloxy)-phenyl]-3-{4-[4-(2,2-dimethyl-propyl)-piperazin-1-yl]-3-trifluoromethyl-phenyl}-urea;
1-[4-(6-Chloro-pyrimidin-4-yloxy)-phenyl]-3-[4-(1-methyl-piperidin-4-ylmethoxy)-3-trifluoromethyl-phenyl]-urea;
1-[4-(6-Chloro-pyrimidin-4-yloxy)-phenyl]-3-[4-(1-methyl-piperidin-4-yloxy)-3-trifluoromethyl-phenyl]-urea;
N-[4-(6-Chloro-pyrimidin-4-yloxy)-phenyl]-N′-{4-[2-(4-ethyl-piperazin-1-yl)-ethyl]-3-trifluoromethyl-phenyl}-urea;
3-{3-[4-(6-Chloro-pyrimidin-4-yloxy)-phenyl]-ureido}-5-trifluoromethyl-benzamide;
3-{3-[4-(6-Methylamino-pyrimidin-4-yloxy)-phenyl]-ureido}-5-trifluoromethyl-benzamide;
3-{3-[4-(6-Azido-pyrimidin-4-yloxy)-phenyl]-ureido}-5-trifluoromethyl-benzamide;
3-{3-[4-(6-Amino-pyrimidin-4-yloxy)-phenyl]-ureido}-5-trifluoromethyl-benzamide;
N-[4-(6-Methylamino-pyrimidin-4-yloxy)-phenyl]-N′-(3-aminomethyl-5-trifluoromethyl-phenyl-urea;
3-{3-[4-(6-Chloro-pyrimidin-4-yloxy)-phenyl]-ureido}-N-methyl-5-trifluoromethyl-benzamide;
3-{3-[4-(6-Methylamino-pyrimidin-4-yloxy)-phenyl]-ureido}-N-methyl-5-trifluoromethyl-benzamide;
3-{3-[4-(6-Azido-pyrimidin-4-yloxy)-phenyl]-ureido}-N-methyl-5-trifluoromethyl-benzamide;
3-{3-[4-(6-Amino-pyrimidin-4-yloxy)-phenyl]-ureido}-N-methyl-5-trifluoromethyl-benzamide;
N-[4-(6-Methylamino-pyrimidin-4-yloxy)-phenyl]-N′-(3-methylaminomethyl-5-trifluoromethyl-phenyl)-urea;
N-[4-(2-Amino-pyrimidin-4-yloxy)-phenyl]-N′-[4-(4-isopropylpiperazin-1-ylmethyl)-3-trifluoromethyl-phenyl]-urea;
N-[4-(2-Methylamino-pyrimidin-4-yloxy)-phenyl]-N′-[4-(4-isopropylpiperazin-1-ylmethyl)-3-trifluoromethyl-phenyl]-urea;
N-[4-(2-Chloro-pyrimidin-4-yloxy)-phenyl]-N′-[4-(dimethylamino-methyl)-3-trifluoromethyl-phenyl]-urea;
N-[4-(2-Methylamino-pyrimidin-4-yloxy)-phenyl]-N′-[4-(4-dimethylamino-methyl)-3-trifluoromethyl-phenyl]-urea;
N-[4-(2-Methylamino-pyrimidin-4-yloxy)-phenyl]-N′-[4-(4-tert-butylpiperazinyl-methyl)-3-trifluoromethyl-phenyl]-urea;
N-[4-(2-Amino-pyrimidin-4-yloxy)-phenyl]-N′-[4-(4-tert-butylpiperazinyl-methyl)-3-trifluoromethyl-phenyl]-urea;
1-[4-(6-Amino-pyrimidin-4-yloxy)-phenyl]-3-[3-(6-methyl-pyridin-2-yl)-5-trifluoromethyl-phenyl]-urea;
1-[4-(6-Amino-pyrimidin-4-yloxy)-phenyl]-3-(4-pyridin-2-yl-3-trifluoromethyl-phenyl)-urea;
1-[4-(6-Amino-pyrimidin-4-yloxy)-phenyl]-3-(4-pyridin-3-yl-3-trifluoromethyl-phenyl)-urea;
1-[4-(6-Amino-pyrimidin-4-yloxy)-phenyl]-3-(4-pyridin-4-yl-3-trifluoromethyl-phenyl)-urea;
1-[4-(6-Amino-pyrimidin-4-yloxy)-phenyl]-3-[4-(6-methyl-pyridin-2-yl)-3-trifluoromethyl-phenyl]-urea;
1-[4-(6-Methylamino-pyrimidin-4-yloxy)-phenyl]-3-(4-pyridin-2-yl-3-trifluoromethyl-phenyl)-urea;
1-[4-(6-Methylamino-pyrimidin-4-yloxy)-phenyl]-3-(4-pyridin-3-yl-3-trifluoromethyl-phenyl)-urea;
1-[4-(6-Methylamino-pyrimidin-4-yloxy)-phenyl]-3-(4-pyridin-4-yl-3-trifluoromethyl-phenyl)-urea;
1-[4-(6-Methylamino-pyrimidin-4-yloxy)-phenyl]-3-[4-(6-methyl-pyridin-2-yl)-3-trifluoromethyl-phenyl]-urea;
N-[4-(6-Chloro-pyrimidin-4-yloxy)-phenyl]-N′-[4-(4-ethylpiperazin-1-ylmethyl)-3-chloro-phenyl]-urea;
1-[4-(2-Amino-pyrimidin-4-yloxy-phenyl]-3-(4-piperazin-1-ylmethyl-3-trifluoromethyl-phenyl)-urea;
1-[4-(2-Methylamino-pyrimidin-4-yloxy-phenyl]-3-(4-piperazin-1-ylmethyl-3-trifluoromethyl-phenyl)-urea;
N-(6-{4-[3-(3-Trifluoromethyl-phenyl)-ureido]-phenoxy}-pyrimidin-4-yl)-acetamide;
N-(6-{4-[3-(4-Morpholin-4-yl-3-trifluoromethyl-phenyl)-ureido]-phenoxy}-pyrimidin-4-yl)-acetamide;
6-(4-{3-[4-(4-Ethyl-piperazin-1-ylmethyl)-3-trifluoromethyl-phenyl]-ureido}-phenoxy)-pyrimidin-4-yl]-carbamic acid methyl ester;
1-[4-(2-Acetylamino-pyrimidin-4-yloxy)-phenyl]-3-[4-(4-methyl-piperazin-1-ylmethyl)-3-trifluoromethyl-phenyl]-urea;
3-[3-(4-{6-[4-(tert-Butyl-dimethyl-silanyloxy)-phenylamino]-pyrimidin-4-yloxy}-phenyl)-ureido]-5-trifluoromethyl-benzamide;
1-(3′-Chloro-2-trifluoromethyl-biphenyl-4-yl)-3-{4-[2-(4-dimethylamino-butylamino)-pyrimidin-4-yloxy]-2-methyl-phenyl}-urea;
1-(3′-Bromo-2-trifluoromethyl-biphenyl-4-yl)-3-{4-[2-(4-dimethylamino-butylamino)-pyrimidin-4-yloxy]-2-methyl-phenyl}-urea;
1-(4′-Chloro-2-trifluoromethyl-biphenyl-4-yl)-3-{4-[2-(4-dimethylamino-butylamino)-pyrimidin-4-yloxy]-2-methyl-phenyl}-urea;
1-(4′-Bromo-2-trifluoromethyl-biphenyl-4-yl)-3-{4-[2-(4-dimethylamino-butylamino)-pyrimidin-4-yloxy]-2-methyl-phenyl}-urea;
1-(3′-Chloro-2-trifluoromethyl-biphenyl-4-yl)-3-{4-[2-(4-dimethylamino-butylamino)-pyrimidin-4-yloxy]-2-trifluoromethyl-phenyl}-urea;
1-(3′-Bromo-2-trifluoromethyl-biphenyl-4-yl)-3-{4-[2-(4-dimethylamino-butylamino)-pyrimidin-4-yloxy]-2-trifluoromethyl-phenyl}-urea;
1-(4′-Chloro-2-trifluoromethyl-biphenyl-4-yl)-3-{4-[2-(4-dimethylamino-butylamino)-pyrimidin-4-yloxy]-2-trifluoromethyl-phenyl}-urea;
1-(4′-Bromo-2-trifluoromethyl-biphenyl-4-yl)-3-{4-[2-(4-dimethylamino-butylamino)-pyrimidin-4-yloxy]-2-trifluoromethyl-phenyl}-urea;
1-(3′-Chloro-2-trifluoromethyl-biphenyl-4-yl)-3-{4-[2-(4-dimethylamino-butylamino)-pyrimidin-4-yloxy]-phenyl}urea;
1-(4′-Chloro-2-trifluoromethyl-biphenyl-4-yl)-3-{4-[2-(4-dimethylamino)-pyrimidin-4-yloxy]-phenyl}-urea;
1-(4′-Bromo-2-trifluoromethyl-biphenyl-4-yl)-3-{4-[2-(4-dimethylamino-butylamino)-pyrimidin-4-yloxy]-phenyl}-urea;
1-{4-[2-(3-Methoxy-phenylamino)-pyrimidin-4-yloxy]-phenyl}-3-[4-(4-methyl-piperazin-1-ylmethyl)-3-trifluoromethyl-phenyl]-urea;
1-2-Methyl-4-{2-[4-(4-methyl-piperazin-1-yl)-phenylamino]-pyrimidin-4-yloxy}-phenyl)-3-(3-trifluoromethyl-phenyl)-urea;
1-{4-[6-(5-Chloro-2-methoxy-phenylamino)-pyrimidin-4-yloxy]-phenyl}-3-(4-morpholin-4-yl-3-trifluoromethyl-phenyl)-urea;
1-{4-[6-(4-Methyl-piperazin-1-yl)-pyrimidin-4-yloxy]-phenyl}-3-(4-morpholin-4-yl-3-trifluoromethyl-phenyl)-urea;
1-[4-(6-Dimethylamino-pyrimidin-4-yloxy)-phenyl]-3-(4-morpholin-4-yl-3-trifluoromethyl-phenyl)-urea;
N,N-Dimethyl-4-(6-{4-[3-(4-morpholin-4-yl-3-trifluoromethyl-phenyl)-ureido]-phenoxy}-pyrimidin-4-ylamino)-benzamide;
1-{4-[6-(2-Methoxy-5-methyl-phenylamino)-pyrimidin-4-yloxy]-phenyl}-3-(4-morpholin-4-yl-3-trifluoromethyl-phenyl)-urea;
1-{4-[6-(2-Methoxy-5-nitro-phenylamino)-pyrimidin-4-yloxy]-phenyl}-3-(4-morpholin-4-yl-3-trifluoromethyl-phenyl)-urea;
1-{4-[6-(2,5-Dimethoxy-phenylamino)-pyrimidin-4-yloxy]-phenyl}-3-(4-morpholin-4-yl-3-trifluoromethyl-phenyl)-urea;
1-{4-[6-(2,5-Dimethoxy-phenylamino)-pyrimidin-4-yloxy]-phenyl}-3-(4-morpholin-4-yl-3-trifluoromethyl-phenyl)-urea;
N,N-Diethyl-4-methoxy-3-(6-{4-[3-(4-morpholin-4-yl-3-trifluoromethyl-phenyl)-ureido]-phenoxy}-pyrimidin-4-ylamino)-benzenesulfonamide;
1-{4-[6-(2-Methoxy-phenylamino)-pyrimidin-4-yloxy]-phenyl}-3-(4-morpholin-4-yl-3-trifluoromethyl-phenyl)-urea;
or a salt thereof.

19. A pharmaceutical composition comprising an N-[4-(pyrimidin-4-yloxy)-phenyl]-N′-phenyl-urea derivative selected from the group consisting of the compounds of claim 17.

20. A pharmaceutical composition comprising an N-[4-(pyrimidin-4-yloxy)-phenyl]-N′-phenyl-urea derivative selected from the compounds of claim 18.

21. A method of treating a protein kinase dependent disease depending on any one or more of the protein tyrosine kinases selected from c-Abl, Bcr-Abl, Flt-3, RET, VEGF-R and/or Tek comprising administering a compound according to claim 17.

22. A method of treating a protein kinase dependent disease depending on any one or more of the protein tyrosine kinases selected from: c-Abl, Bcr-Abl, Flt-3, RET, VEGF-R and/or Tek, comprising administering a compound according to claim 18.

23. A method according to claim 21 wherein the protein kinase is Flt-3.

24. A method according to claim 22 wherein the protein kinase is Flt-3.
